SEQUENCE LISTING (49 sequences)

SEQ ID NO: 1 - DNA, 9 bp, Human endogenous retrovirus K
tttctcccc

SEQ ID NO: 2 - DNA, 9 bp, Human endogenous retrovirus K
ccctctccc

SEQ ID NO: 3 - DNA, 10 bp, Human endogenous retrovirus K
ccccctcttt

SEQ ID NO: 4 - DNA, 11 bp, Human endogenous retrovirus K
tttctttctct

SEQ ID NO: 5 - DNA, 14 bp, Human endogenous retrovirus K
tctttttcttttcc

SEQ ID NO: 6 - DNA, 9 bp, Human endogenous retrovirus K
ggggagaaa

SEQ ID NO: 7 - DNA, 9 bp, Human endogenous retrovirus K
gggagaggg

SEQ ID NO: 8 - DNA, 10 bp, Human endogenous retrovirus K
aaagaggggg

SEQ ID NO: 9 - DNA, 11 bp, Human endogenous retrovirus K
agagaaagaaa

SEQ ID NO: 10 - DNA, 14 bp, Human endogenous retrovirus K
ggaaaagaaaaaga

SEQ ID NO: 11 - DNA, 20 bp, Human endogenous retrovirus K
caaggtttctccccatgtga

SEQ ID NO: 12 - DNA, 20 bp, Human endogenous retrovirus K
tgctgaccctctcccacaat

SEQ ID NO: 13 - DNA, 20 bp, Human endogenous retrovirus K
acacatccccctctttgaga

SEQ ID NO: 14 - DNA, 20 bp, Human endogenous retrovirus K
ccttatttctttctctatac

SEQ ID NO: 15 - DNA, 20 bp, Human endogenous retrovirus K
gtgtctttttcttttccaaa

SEQ ID NO: 16 - DNA, 20 bp, Homo sapiens
cggatatgatatctataccg

SEQ ID NO: 17 - DNA, 20 bp, Human endogenous retrovirus K
ctgaggcaattgcaggagtt

SEQ ID NO: 18 - DNA, 20 bp, Human endogenous retrovirus K
gctgtctcttcggagctgtt

SEQ ID NO: 19 - DNA, 20 bp, Human endogenous retrovirus K
tcacatggaaacaggcaaaa

SEQ ID NO: 20 - DNA, 20 bp, Human endogenous retrovirus K
aggtacatgcgtgacatcca

SEQ ID NO: 21 - DNA, 24 bp, Human endogenous retrovirus K
agcaggtcaggtgcctgtaacatt

SEQ ID NO: 22 - DNA, 24 bp, Human endogenous retrovirus K
tggtgccgtaggattaagtctcct

SEQ ID NO: 23 - DNA, 20 bp, Human endogenous retrovirus K
gggccaattatgcttaccaa

SEQ ID NO: 24 - DNA, 20 bp, Human endogenous retrovirus K
atgggctgatctggctctaa

SEQ ID NO: 25 - DNA, 25 bp, Human endogenous retrovirus K
ctggtccacgcacggccgaagcatg

SEQ ID NO: 26 - DNA, 25 bp, Human endogenous retrovirus K
aaaaggacgacttaatagagccaat

SEQ ID NO: 27 - DNA, 19 bp, Human endogenous retrovirus K
caagattgggtcccctcac

SEQ ID NO: 28 - DNA, 19 bp, Human endogenous retrovirus K
cctatggggtctttccctc

SEQ ID NO: 29 - DNA, 20 bp, Homo sapiens
tgcaccaccaactgcttagc

SEQ ID NO: 30 - DNA, 21 bp, Homo sapiens
ggcatggactgtggtcatgag

SEQ ID NO: 31 - DNA, 19 bp, Artificial Sequence
  Synthetic primer
gtgtgcctgttttgtctgc

SEQ ID NO: 32 - DNA, 22 bp, Artificial Sequence
  Synthetic primer
cacgatctggtcccttttactc

SEQ ID NO: 33 - DNA, 21 bp, Homo sapiens
aggtcggtgtgaacggatttg

SEQ ID NO: 34 - DNA, 19 bp, Homo sapiens
ggggtcgttgatggcaaca

SEQ ID NO: 35 - DNA, 21 bp, Homo sapiens
tcctgctcaacttcctgtcga

SEQ ID NO: 36 - DNA, 24 bp, Homo sapiens
cacaggtcaaacctcctaggaatg

SEQ ID NO: 37 - DNA, 28 bp, Homo sapiens
cgagacgctaccatggctatcgctgtag

SEQ ID NO: 38 - DNA, 20 bp, Mus musculus
accagctggctgacctgtag

SEQ ID NO: 39 - DNA, 20 bp, Mus musculus
ggcagcttcatctgttcctc

SEQ ID NO: 40 - DNA, 20 bp, Human endogenous retrovirus K
gtttgtctgctgaccctctc

SEQ ID NO: 41 - DNA, 21 bp, Human endogenous retrovirus K
cctgtgggtgtttctcgtaag

SEQ ID NO: 42 - DNA, 20 bp, Human endogenous retrovirus K
ggaaagccaggtattgtcca

SEQ ID NO: 43 - DNA, 20 bp, Human endogenous retrovirus K
ctcctcagcacagacccttt

SEQ ID NO: 44 - DNA, 20 bp, Human endogenous retrovirus K
gggcagcaatactgctttgt

SEQ ID NO: 45 - DNA, 20 bp, Human endogenous retrovirus K
ttctcaaagagggggatgtg

SEQ ID NO: 46 - DNA, 20 bp, Human endogenous retrovirus K
cacatccccctctttgagaa

SEQ ID NO: 47 - DNA, 20 bp, Human endogenous retrovirus K
ctcgtaaggtgggacgagag

SEQ ID NO: 48 - DNA, 20 bp, Homo sapiens
tactagcggttttacgggcg

SEQ ID NO: 49 - DNA, 24 bp, Homo sapiens
tcgaacaggaggagcagagagcga

(19) United States
(12) Patent Application Publication
(10) Pub. No.: US 2019/0038659 A1
(43) Pub. Date: Feb. 7, 2019

(54) METHODS OF TREATING AND PREVENTING AMYOTROPHIC LATERAL SCLEROSIS

(71) Applicant: THE USA, as represented by the Secretary, Department of Health and Human Services, Bethesda, MD (US)

(72) Inventors: Avindra NATH, Ellicott City, MD (US); Wenxue LI, Potomac, MD (US); Joseph Perry STEINER, Mount Airy, MD (US); Myoung Hwa LEE, Silver Spring, MD (US); Lisa HENDERSON, Manassas, VA (US); Richa TYAGI, Seattle, WA (US)

(21) Appl. No.: 15/764,647

(22) PCT Filed: Sep. 29, 2016
(86) PCT No.: PCT/US2016/054519

§ 371 Date: Mar. 29, 2018

Related U.S. Application Data
(60) Provisional application No. 62/234,419, filed on Sep. 29, 2015.

(51) Int. Cl. A61K 31/7105; A61P 31/12; A61P 25/28; A61K 31/711

(52) U.S. Cl. CPC A61K 31/7105; A61K 31/711; A61P 25/28; A61P 31/12

(57) ABSTRACT

Methods of treating amyotrophic lateral sclerosis (ALS) or preventing the progression of ALS. Compounds useful in these therapeutic methods include anti-retroviral compounds and RNA interference (RNAi) constructs.

### REFERENCE TO SEQUENCE LISTING

This application contains a Sequence Listing submitted as an electronic text file named “6137NINDS-1-PCT_Sequence_Listing_ST25.txt”, having a size in bytes of 9 kb, and created on Sep. 28, 2016. The information contained in this electronic file is hereby incorporated by reference in its entirety pursuant to 37 CFR § 1.52(e)(5).

### TECHNICAL FIELD

The disclosure relates to novel methods for treating amyotrophic lateral sclerosis (ALS). More specifically, it relates to the association between the transactivation of an endogenous retrovirus in an individual and subsequent development of ALS in the individual. The disclosure discloses methods of modulating transcription of endogenous retroviral genes thereby preventing of treating ALS.

### BACKGROUND

Amyotrophic lateral sclerosis (ALS), commonly referred to as Lou Gehrig's disease or clinically as motor neuron disease, is a fatal, neurodegenerative disease characterized by loss of motor neurons. The classic clinical symptoms of ALS are due to the progressive loss of both upper motor neurons (UMN) in the cerebral cortex, and lower motor neurons (LMN) in the brain and spinal cord. More recently, however, ALS has come to be recognized as a multi-system, degenerative disease, in which motor neurons are especially, but not exclusively, involved. Examples of symptoms resulting from motor neuron degeneration include muscle cramping, muscle twitch (fasciculation), muscle atrophy, muscle weakness, slow movement, spasticity, loss of fine muscle movement, increased deep tendon reflex, and the inability to regulate laughing and/or crying. Symptoms of degeneration of non-motor neurons include loss of executive function (cognitive control), frontotemporal dementia (FTD), Parkinsonism and sensory loss. Currently, ALS is diagnosed based on the presence of one or more of the symptoms listed above, using the El Escorial Criteria (Brooks B R, Miller R G, Swash M, Munsat T L. El Escorial revisited: revised criteria for the diagnosis of amyotrophic lateral sclerosis. Amyotroph Lateral Scler Other Motor Neuron Disord. 2000 December; 1(5):293-9).

The incidence of ALS is approximately 1-3 per 100,000 individuals, and is consistent across diverse populations. An individual's lifetime risk of developing ALS is 1 in 300-1000.

To date, mutations in more than 20 genes have been identified in patients with ALS, or ALS-like phenotypes. However, the clinical manifestations associated with each of the known genes, and specific mutations in such genes, show broad clinical heterogeneity. For example, the age of onset can vary by decades, the phenotype can vary from pure LMN syndrome to pure FTD, and progression can range from rapid to prolonged survival. Thus, while an association between the identified genes and the development of ALS has been shown, it is clear that other factors, for example as-yet undiscovered genes, other genetic elements, or perhaps even environmental elements, have a role in the development and progression of ALS.

One such factor may be endogenous retroviruses. For example, U.S. Patent Publication No. 2006/0160087, the disclosure of which is incorporated herein by reference in its entirety, teaches an association between the development or progression of ALS and the presence or absence of proteins from the endogenous retrovirus HERV-K in a biological sample from an individual. The phrase human endogenous retrovirus (HERV) is a broad heading for viruses from numerous families of retroviruses that were able to infect human germline cells during the course of human evolution. Over time, the genomes of these infecting retroviruses were integrated into the human genome with the result that the integrated HERV genomes were transmitted to progeny humans in a Mendelian fashion, thereby overriding the need to spread by exogenously acquired infection. This process of retroviral integration resulted in modern humans having at least 31 independently acquired HERV families in their genomes (for a review, see Douville, and Nath, Human endogenous retroviruses and the nervous system, Handbook of Clinical Neurology, Vol. 123, 3^{rd} series, pages 465-485, 2014). As a consequence of this, approximately 8% of the human genome is derived from retroviral-like elements.

While the sequences of different types of HERVs vary from one another, a basic retroviral genomic organization exists. The HERV genomes contains nucleic acid sequences encoding the four essential Retroviridae genes, 5′-gag-pro, pol-env-3′. The gag gene encodes the Matrix (MA), capsid (CA) and Nucleocapsid (NC) proteins. The pro gene encodes the viral protease (PR), while the pol gene encodes the reverse transcriptase (RT) and integrase (IN) proteins, and the env gene encodes the envelope protein having surface (SU) and transmembrane (TM) subunits. Following internalization of the retrovirus, the co-packaged RT enzyme uses cellular tRNA as a primer to convert viral RNA into a double-stranded (ds) DNA genome, which has long-terminal repeats (LTRs) at both ends. The viral integrase then facilitates integration of the viral genome into a random location in the host chromosomal DNA. It should be noted that the LTRs are also incorporated into the host chromosomal DNA so that the integrated retroviral coding sequences are flanked by LTR sequences. The integrated LTRs have viral promoter function and serve to regulate transcription of the integrated viral genes. Interestingly, the viral LTRs have been shown to regulate cellular genes as well. The structure of the LTRs is known to those skilled in the art (the tON may comprise a sequence selected from the group consisting of SEQ ID NO:6, SEQ ID NO:7, SEQ ID NO:8, SEQ ID NO:9, and SEQ ID NO:10). Each LTR contains a U3 (unique 3′) region, an R (repeated) region, and a U5 (unique) region. These regions contain genetic elements that bind viral and host proteins, thereby affecting transcription of the viral genes as well as host genes (Buzdin, et al. J. of Virol, No. 2006, 10752-62).

While most HERV viruses have accumulated numerous mutations that render them defective, it is becoming increasingly apparent that endogenous retroviral sequences may be expressed under select pathological conditions. For example, U.S. Patent Publication No. 2014/0099324 teaches that HERV-K mRNA is frequently expressed in breast cancer cells whereas HERV-E mRNA is expressed in prostate cancer. US20140135384 also teaches that HERV-K transcripts are found in prostate cancer. US20150246067 teaches that there is a dramatic increase in the level of HERV-K RNA found in the blood of lymphoma patients. Similarly, US2014/0377758 teaches that certain endogenous transposons become more activated as a result of the normal aging process. U.S. Pat. No. 7,666,420 teaches that endogenous retroviruses have been found to have a role in the development and diabetes, schizophrenia and multiple sclerosis. All of these publications are incorporated herein by reference in their entirety.

Multiple complete sequences of the most recently acquired HERV-K are present in the human genome. HERV-K may be expressed in the brain of patients with amyotrophic lateral sclerosis (ALS) and reverse transcriptase activity can be found in the blood and brain tissue of these patients. But the role of HERV-K in the pathophysiology of this disease remains unknown. ALS is a progressive neurodegenerative disease and is universally fatal except in some patients with human immunodeficiency virus infection where an ALS-like syndrome can be reversed by antiretroviral drugs. However, an extensive search for exogenous retroviruses in ALS has not been successful. The present disclosure demonstrates and association between activation of HERV-K and the development of ALS. The present disclosure also discloses methods of treatment based on the disclosed association.

### SUMMARY

This disclosure demonstrates that human endogenous retrovirus-K (HERV-K) is expressed in neurons of a subpopulation of patients with amyotrophic lateral sclerosis (ALS). The inventors have discovered that envelope protein of this virus surprisingly causes degeneration of neurons, and transgenic animals expressing this protein develop an ALS-like syndrome caused by nucleolar dysfunction in motor neurons. This disclosure therefore provides therapeutic compositions and treatment methods useful in reducing or eliminating reactivation and/or expression of this virus in order to treat or prevent ALS.

One method of treating or preventing amyotrophic lateral sclerosis (ALS) in a subject provided by this disclosure includes administering to a subject diagnosed with ALS, or a subject at risk for developing ALS, one or more compounds that reduce the viral load of HERV-K retrovirus in the subject. The subject may have greater than 100 copies of HERV-K gag RNA/ml of whole blood, or greater than 1000 copies of HERV-K gag RNA/ml of whole blood, prior to initiating the administration of the one or more compounds. The subjects to be treated may be confirmed as patients that do not have an HIV infection. The compounds administered may include antiretroviral compounds, which may include one or more compounds that are therapeutically effective as retroviral reverse transcriptase inhibitors, protease inhibitors, and integrase inhibitors. These inhibitors may include one or more compounds selected from Abacavir, Zidovudine, Lamivudine, Stavudine, Tenofovir, Efavirenz, Etravirine, Nevirapine, Lopinavir, Tipranavir, Saquinavir, Nelfinavir, Amprenavir, Darunavir, Indinavir, and Atazanavir, and Raltegravir. A specific cocktail of inhibitors that is contemplated for administration in these methods includes Darunavir, Ritonavir, Zidovudine and Raltegravir. In methods of treating an ALS patient, the selection of the appropriate antiretroviral drug treatment may be based on the initial detection of the HERV-K retrovirus.

In these therapeutic methods, the compounds selected for administration may include at least one compound that has an IC90 (concentration of drug needed to inhibit 90% of HERV-K retroviral growth) of less than 0.8 μM, less than 0.1 μM, or less than 50 nM.

In these treatment methods, the HERV-K retrovirus may be reduced to below detectable levels in the subject's blood, which can include reducing the HERV-K retrovirus to undetectable levels in a blood sample from the subject.

In these therapeutic methods, the compounds selected for administration may include at least one compound that binds to a HERV-K polynucleotide sequence, which may be an HERV-K genomic nucleic acid molecule. These compounds may bind a polynucleotide sequence in the HERV-K LTR region. The binding of these compounds to the HERV-K polynucleotide sequence may inhibit binding of a transcription factor to a transcription factor binding site in the HERV-K LTR. This includes compounds that bind directly to the transcription factor binding site in the HERV-K LTR. In these methods, the transcription factor binding site is a TDP-43 binding site. In these methods, the transcription factor binding site may include the nucleotide sequence: CCCTCTCCC (SEQ ID NO:2). In certain methods, the transcription factor binding site may include a nucleotide sequence comprising a sequence selected from the group consisting of SEQ ID NO:1, SEQ ID NO:2, SEQ ID NO:3, SEQ ID NO:4, SEQ ID NO:5. In certain methods, the transcription factor binding site may include a nucleotide sequence comprising a sequence selected from the group consisting of SEQ ID NO:11, SEQ ID NO:21, SEQ ID NO:13, SEQ ID NO:14, SEQ ID NO:15. In certain methods, the tON may comprise a sequence selected from the group consisting of SEQ ID NO:6, SEQ ID NO:7, SEQ ID NO:8, SEQ ID NO:9 and SEQ ID NO:10. Binding of these compounds to the HERV-K polynucleotide sequence may act to inhibit transcription and/or block transcription of one or more HERV-K nucleic acid sequences.

In these methods, the HERV-K polynucleotide may be an mRNA molecule transcribed from the HERV-K genome. In these instances, binding of the one or more compound to the HERV-K mRNA may reduce or prevent translation of the bound HERV-K mRNA. Alternatively, or additionally, the binding of the one or more compounds to the HERV-K mRNA may increase degradation of the bound HERV-K mRNA.

In these methods, the one or more compounds may bind to a protein that specifically binds a polynucleotide sequence in the HERV-K genome. Such proteins may be a transcription factor, which may include, specifically, the transcription factor TDP-43. In these methods, the binding of the one or more compounds to the protein inhibits binding of the protein to the polynucleotide sequence. This binding may inhibit transcription of one or more HERV-K nucleic acid sequences.

In these methods, the one or more compounds may be a therapeutic oligonucleotide (tON). The tON may be administered as a naked nucleic acid molecule. Alternatively, or additionally, the tON administered may be complexed with a lipid or a DNA molecule. The tON may also be administered within a viral vector comprising or expressing the tON. The viral vector may be selected from the group consisting of an adenovirus vector, an adeno-associated virus vector, a lentivirus vector, and a poxvirus vector.

In these methods, the subjects selected for treatment may include individuals recently diagnosed with ALS, or individuals believed to be at risk of developing ALS without a confirmed diagnosis of ALS. In these instances, the therapeutic methods of this disclosure may include preventing the development or progression of ALS in the subject.

In view of these useful therapeutic methods, this disclosure provides therapeutic oligonucleotides (tON) capable of reducing the level of HERV-K retrovirus, or the level of at least one HERV-K protein, when the tON is administered to a subject expressing the HERV-K retrovirus. The tON may be an antisense RNA, an inhibitory RNA (iRNA), a small hairpin RNA (shRNA), a microRNA (miRNA), and an aptamer. The tON may be chemically modified to increase its stability, increase its solubility, and/or increase its resistance to degradation. The tON may be prepared for administration as a naked nucleic acid molecule. The tON may also be prepared for administration as a complex with a lipid or a DNA molecule and therefore, such lipid complex including one or more tON is encompassed by this disclosure. This disclosure also provides a viral vector comprising or expressing these tON. The viral vector may be selected from the group consisting of an adenovirus vector, an adeno-associated virus vector, a lentivirus vector, and a poxvirus vector.

The foregoing will become more apparent from the following detailed description, which proceeds with reference to the accompanying figures.

### DESCRIPTION OF FIGURES

FIGS. 1A-1H demonstrate HERV-K expression in brain of ALS patients. FIG. 1A shows the HERV-K genome regions amplified by PCR. FIGS. 1B-1D shows that all HERV-K genes were significantly elevated in ALS patients (n=11, ALS and n=16, controls). Values represent mean±SEM. Significance was determined by unpaired Student's t test. Variances were significantly different between groups. FIGS. 1E-1G show Pearson correlation analyses revealed positive correlations between the mRNA levels of HERV-K env, pol, and gag from autopsy brain cortical tissues. Pearson's correlation coefficients were used to quantify the linear relationship between two variables. FIG. 1H shows the levels of HERV mRNA in control subjects relative to ALS patients.

FIGS. 2A-2G demonstrate that HERV-K env induced-neuronal toxicity in vitro. The HERV-K env or the entire HERV-K genome was transfected into pluripotent stem cell-derived human neurons expressing td-Tomato (fluorescent marker to label the neurons) and morphological changes noted 24 hours post-transfection. pcDNA were used as a control. Scale bars are 200 μm and 50 μm. Total cell counts (FIG. 2A) and neurite length (FIG. 2B). Values represent mean±SEM from three independent experiments. Significance was determined by one-way ANOVA followed by Newman Keuls post hoc comparison. Variances were significantly different between groups. FIG. 2C shows the total cell count from pluripotent stem cell-derived human neurons transfected with varying concentrations of control DNA, HERV-K env encoding DNA or the entire HERV-K genome. FIG. 2D shows the neurite length of pluripotent stem cell-derived human neurons transfected with varying concentrations of control DNA, HERV-K env encoding DNA or the entire HERV-K genome. FIG. 2E shows the relative levels of HERV-K gag and env mRNA in transfected cells. FIGS. 2D and 2E show the endogenous HERV-K expression was induced using the CRISPR/Cas9 system. Stem cell derived human neurons were transduced with a lentiviral construct encoding Cas9 fused to transcription activation domain VP64 for 24 hours. Cells were either mock treated (Cas9 alone) or transduced with guide RNA targeting the HERV-K promoter (sgRNA 8). Total cell counts from transfected cells (FIG. 2D) and mean neurite fiber length from transfected cells (FIG. 2E), were collected. Values represent mean±SEM from three independent experiments. Significance was determined by unpaired Student's t test. FIG. 2H shows total cell count in neurons treated with 3-nitropropionic acid (3NP), N-methyl-D-aspartate (NMDA), or hydrogen peroxide (H₂O₂). FIG. 2I shows neurite length in neurons treated with 3-nitropropionic add (3NP), N-methyl-D-aspartate (NMDA), or hydrogen peroxide (H₂O₂). FIG. 2G shows relative levels of HERV-K viral transcripts in neurons treated with 3-nitropropionic acid (3NP). N-methyl-D-aspartate (NMDA), or hydrogen peroxide (H₂O₂).

FIGS. 3A-3H demonstrate HERV-K induced-neuronal toxicity in vivo. Coronal sections of wild type (wt) and HERV-K env transgenic (tg) mice were immunostained and examined for HERV-K env. Values represent mean±SEM. Significance was determined by unpaired Student's t test. FIG. 3A shows the number of neuritic beads/100 μM of embryonic mouse brain from mice in utero electroporated with HERV-K env gene. FIG. 3B shows the relative levels of HERV-K env transcripts in post-mortem brain tissue of ALS patients, and in transgenic mice. FIG. 3C shows the total dendrite length in coronal neurons from wild-type and transgenic mice. FIG. 3D shows the mean dendrite branch number in coronal neurons from wild-type and transgenic mice. FIG. 3E shows the number of intersections in coronal neurons from wild-type and transgenic mice. FIG. 3F shows the number of dendritic spines in coronal neurons from wild-type and transgenic mice. FIGS. 3G and 3H shows the spine density in coronal neurons from wild-type and transgenic mice.

FIGS. 4A-4J show HERV-K env expression in injury to lower motor neurons. Brain sections from wild type (wt) and HERV-K env transgenic (tg) mice were immunostained and examined for HERV-K env, GFAP, NeuN (as a marker for neurons), Ctip2 (as a marker forcorticospinal motor neurons), Satb2 (as a marker for callosal projection neurons in layer 5 of the motor cortex of wt (n=4) and tg (n=3) mice), and nucleophosmin (as a nucleolar marker), and cell numbers noted. Values represent mean±SEM. Significance was determined by unpaired Student's t test. FIG. 4A shows the number of NeuN+ cell/mm³ in brain sections from wild-type and transgenic mice. FIG. 4b shows the number of Ctip2+ cell/mm³ in brain sections from wild-type and transgenic mice. FIG. 4C shows the number of Stab2+ cell/mm³ in brain sections from wild-type and transgenic mice. FIG. 4D shows the thickness of motor cortex (mm) in brain sections from wild-type and transgenic mice. FIG. 4E shows the volume of motor cortex (mm³) in brain sections from wild-type and transgenic mice. FIG. 4F shows the volume of cingulate cortex (mm³) in brain sections from wild-type and transgenic mice. FIG. 4G shows the thickness of corpus callosum (mm) in brain sections from wild-type and transgenic mice. FIG. 4H shows the volume of hippocampus (mm³) in brain sections from wild-type and transgenic mice. FIG. 4I shows the γH2A.X-positive foci in immunostained entorhinal cortex from 6-month-old wt (n=4) and tg (n=4) mice. Numbers of cells with γH2A.X-positive foci were increased in motor cortex of tg mice. Values represent mean±SEM. Significance was determined by unpaired Student's t test. Scale bar is a 20 μm. FIG. 4J shows the concentration of nucleophosmin (NPM) in cytoplasm of cells in the motor cortex of wt (n=4) and tg (n=3) mice. Numbers of cells with NPM localized to the cytoplasm were increased in the motor cortex of tg mice. Values represent mean±SEM. Significance was determined by unpaired Student's t test. Scale bar is a 10 μm

FIGS. 5A-5Q show HERV-K induced-alterations in behavioral and functional analysis of mouse phenotype. FIGS. 5A-5E show open field testing demonstrating that the tg mice were less active than wildtype (wt) animals as determined by decreased path length travelled (FIG. 5A), increased periods of immobility (FIG. 5B), decreased line crossings (FIG. 5C), decreased numbers of rearing (FIG. 5D), and decreased numbers of entries into the center of the field (FIG. 5E). There was progressive decrease inactivity over time. (n=16, wt and n=15, tg at 3 months; n=26, wt and n=24, tg at 6 months). FIG. 5F shows the time to fall on an accelerating rotarod (n=18, wt and n=17, at 3 and 6 months; n=18, wt and n=9, tg at 9 months. The sample size declined at 9 months due to increased death at that age). FIG. 5G shows the clasping score on a tail suspension test (n=18, wt and n=17, tg). FIG. 5H shows the attention span in Y-maze test for wt and tg mice (n=18, wt and n=17, tg at 3 and 6 months; n=18, wt and n=9, tg at 9 months). FIG. 5I shows the reaction time to notice adhesive tapes sticking on the palms of the hind paws of wt mice (n=10, wt and n=10, tg). FIG. 5J shows the time to turn on a 45° angle slope for wt and tg mice (n=8, wt; n=12, tg). FIG. 5K shows the survival time of transgenic animals over 10 months. FIGS. 5L and 5M show the number of action potentials evoked for a range of current injections. FIG. 5N is pooled data values of sEPSC frequency for wild-type and transgenic mice. FIG. 5O is pooled data values of sEPSC amplitude for wild-type and transgenic mice. FIG. 5P is pooled data values of sIPSP frequency for wild-type and transgenic mice. FIG. 5Q is pooled data values of sIPSP amplitude for wild-type and transgenic mice. Values represent mean±SEM and were analyzed by the Mann-Whitney nonparametric test.

FIGS. 6A-6K show HERV-K activation by TDP-43 and identification of binding sites on LTR. Stem cell-derived neurons were transfected with either pcDNA CAT control or TDP-43 expression construct. At 24 hours post-transfection, cells were collected for RNA extraction and qRT-PCR to measure HERV-K transcripts. FIG. 6A shows relative change in HERV-K transcripts in transfected cells. FIGS. 6B and 6C show HERV-K plasmid co-transfected with CAT (control), Tat, TDP-43, or Tat and TDP-43 in HeLa cells and at 24 hours post-transfection; reverse transcriptase activity (HERV-K RT) was measured in culture supernatants by PERT assay (FIG. 6B), and levels of HERV-K transcripts were measured using RT-PCR and expressed as fold change compared to CAT control (FIG. 6C). FIG. 6D shows Luciferase activity in human neurons co-transfected with HERV-K LTR-MetLuc plasmid and CAT, Tat, TDP-43, or Tat and TDP-43. FIG. 6E shows HERV-K expression in human neurons following knockdown of endogenous TDP-43 with siRNA. FIG. 6F shows the putative TDP-43 binding sites in HERV-K LTR reported relative to the first base of the LTR. FIG. 6G shows TDP-43 binding sites on HERV-K LTR. (H) Relative binding of TDP-43 to regions of HERV-K LTR. FIG. 6I shows binding of TDP-43 to biotinylated oligonucleotides derived from the putative binding sites under low or high-salt conditions. FIG. 6J shows the quantification of the results obtained in FIG. 6H. Values represent mean±SEM from three independent experiments. Significance was determined by unpaired Student's t test. FIG. 6K shows the relative binding of C-terminal repeat domain of RNA polymerase II in cells transfected with HERV-K or HERV-K/TDP-43.

FIGS. 7A-7E show that a consensus HERV-K has the ability to generate active viral particles. FIG. 7A The consensus complete HERV-K genomic sequence was cloned into the pcDNA3.1 vector with HIV-1 Rev resulting in a plasmid termed pCD-HK/Rev. HeLa cells were transfected with the pCD-HK/Rev plasmid in combination with plasmids for HIV-1 Tat. The reverse transcriptase (RT) activity in the culture supernatant was determined by PERT assay (FIG. 7B, left) at 24, 48 and 72 hrs post-transfection. Recombinant HIV RT was diluted serially in culture media and used as an activity standard (FIG. 7B, right). HERV-K RT activity in FIG. 7B, left, was quantified using this standard. FIG. 7C shows a Western blot analyses for HERV-K Gag and Env expression in 293T cells after transfection with pCD-HK/Rev. FIGS. 7D and 7E demonstrate that HIV reverse transcriptase inhibitors can inhibit HERV-K reverse transcriptase. HERV-K supernatant was collected from Hela cells transfected with pCD-HK/Rev plasmid in combination with HIV-1 Tat. Nucleoside HIV-RT inhibitors (FIG. 7D) or Non-nucleotide HIV-RT inhibitors (FIG. 7E) were added in a dose ranging from 0.05 μM-0.25 μM to collected supernatant and PERT assay was performed to quantify HERV-K RT. Any change compared to no treatment is reported as percent inhibition.

FIGS. 8A-8D show that HERV-K Viral replication can be effectively inhibited by Abacavir, Zidovudine and Raltegravir. 293T cells (FIG. 8A, left) and HeLa cells (FIG. 8A right) were infected with HERV-K (HK) or VSV-G pseudotyped HERV-K (vsv-HK) viral particles. Total RNA was extracted 6 days post-infection and quantitative PCR was used to determine HERV-K gag mRNA expression. Glyceraldehyde 3-phosphotate dehydrogenase (GAPDH) was used as internal control and titers were expressed as fold change (FIGS. 8B-8D). Hela cells were infected with 80 pg of VSV-G pseudotyped HERV-K virus and treated with (FIG. 8B) Abacavir (FIG. 8C) Zidovudine or (FIG. 8D) Raltegravir, in a dose ranging from 0.05 μM-0.25 μM. Six days post infection, gag mRNA expression was quantified using quantitative PCR. Gag expression was compared to no treatment as control and expressed as percent inhibition. Data represent mean±SEM of at least 3.

FIGS. 8E-8H show that HERV-K viral particle release can be inhibited by protease inhibitors. Hela cells were transfected with pCD-HK/Rev and HIV-1 Tat plasmids (FIG. 8E) HIV protease inhibitors were added to Hela cells 6 hrs post transfection and the reverse transcriptase (RT) activity in the culture supernatant was determined by PERT assay at 24 hr post-treatment. Darunavir, Lopinavir, Indinavir, Amprenavir or Atazanavir were added to Hela cells 6 hrs post transfection in a 2-fold serial dilution ranging from 31.25 nM to 1 μM and RT activity in the culture supernatant was determined by PERT assay (FIG. 8F) at 48 hr post-treatment. Darunavir (FIG. 8G) and Lopinavir (FIG. 8H) were further screened using 10-fold serial dilution of the compounds, ranging from 0.001 μM-100 μM. Viral supernatant was collected 48 hr post-treatment and analyzed by PERT assay. Any change in Ct (threshold cycle) was compared to vehicle control and reported as percent inhibition. Data represent mean+SEM of at least 3 different experiments.

### DETAILED DESCRIPTION OF THE DISCLOSURE

This disclosure is based on the inventor's discovery of an association between endogenous retroviruses in the human genome and development of amyotrophic lateral sclerosis (ALS). In particular, the inventors have surprisingly discovered that brain tissue from deceased ALS patients contains HERV-K transcripts and proteins, whereas such transcripts and proteins are not found in brain tissue from deceased, non-ALS individuals. Additionally, the inventors have shown that expression of HERV-K proteins causes neurotoxicity, and that mice expressing HERV-K proteins in their neurons developed symptoms consistent with neurodegenerative disease such as ALS, implicating a causal link between expression of the endogenous retrovirus HERV-K and ALS, and providing a potential method of treating or preventing ALS. In view of this discovery, a subject diagnosed as having ALS, or a subject at risk for developing ALS, may be administered one or more compounds that reduce the level of one or more HERV-K proteins, or that reduce the overall viral load of HERV-K, in the subject.

As used in this disclosure, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term amyotrophic lateral sclerosis, or ALS, is understood in the art and as used herein denotes a progressive neurodegenerative disease that affects upper motor neurons, and/or lower motor neurons and/or non-motor neurons. Affected neurons show signs of impairment and/or death. As used herein, ALS includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

As used herein, the terms subject, patient, and individual can be used interchangeably. A subject refers to any vertebrate capable of developing ALS or an ALS-like syndrome. Preferred vertebrates are mammals, including humans, farm animals, sport animals, pets (e.g., dogs, cats, horses) and primates, including non-human primates. In some instances, the subject can also be a laboratory animal, for example in the context of a clinical trial or a potential compound (e.g., drug) screening experiment. A subject of the invention may or may not have another condition or disease in addition to ALS. In one embodiment, the subject treated is not infected with human immunodeficiency virus (HIV).

As used herein, a subject diagnosed as having ALS (an ALS patient, an ALS subject, and the like) is a subject deemed to have ALS by a medical professional (e.g., a physician, a physicians' assistant, a nurse practitioner, a nurse, etc.) using standard diagnostic criteria for ALS. Such diagnosis made be made based on the subject demonstrating ALS-associated symptoms, using for example the El Escorial criteria, an appropriate clinical test for ALS, or combinations thereof. Any combinations thereof may also be used. In addition to observation of ALS-associated symptoms, clinical tests used in diagnosing ALS can include, but are not limited to, electromyography (EMG), a blood test or a genetic test.

Methods of the present invention can also be applied to a subject suspected of having ALS. As used herein, a subject suspected of having ALS displays at least one symptom, or physical characteristic (e.g., clinical test result), associated with ALS, but whom has not been diagnosed as having ALS by a medical professional.

As used herein, a control subject, or normal subject (non-ALS subject), is a subject that is of the same species as, and otherwise comparable to (e.g., similar age, sex, race, etc.), an ALS subject, but whom does not have, or is not suspected of having, ALS. A control subject does not display the full spectrum of symptoms or physical characteristics necessary to be diagnosed as having ALS.

As used in this disclosure, the term treatment, treating, and the like, refers to an approach (e.g., administration of a compound) for the purpose of obtaining beneficial or desired results, including clinical results. For purposes of the present disclosure, beneficial or desired clinical results include, but are not limited to, alleviation or amelioration of one or more symptoms of ALS, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Palliating” a disease or disorder means that the extent and/or undesirable clinical manifestations of a disorder or a disease state are lessened and/or time course of the progression is slowed or lengthened, as compared to not treating the disorder. The term treatment can also refer to prolonging survival as compared to expected survival if a subject did not receive treatment.

The terms administering, administration, administered, and the like, are understood in the art. Any suitable route of administration may be employed for providing a subject, especially a human, with an effective dosage of a compound effective in the treatment of ALS. Examples of suitable means of administration include, but are not limited to, oral, intradermal injection, intramuscular injection, intravenous injection, topical, rectal, ocular, pulmonary, nasal, and the like. Dosage forms can include, but are not limited to, tablets, solutions, dispersions, suspensions, capsules, creams, ointments, aerosols, and the like. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present disclosure is being applied. Suitable carriers, diluents, excipients, and the like, and means of administering compounds of the present disclosure are disclosed in U.S. Patent Publication No. 2014/0113952, published Apr. 24, 2014, which is incorporated herein by reference in its entirety.

As used herein, the terms sufficient, sufficient amount, effective amount, therapeutically effective amount, and the like, refer to an amount (e.g., grams, milligrams, moles, etc.) or concentration (e.g., percent, molar, etc.) of a compound necessary to achieve a desired and/or beneficial result, including a clinical result. A sufficient amount can be administered in one or more administrations. In some embodiments, an effective amount is an amount that reduces the viral load of HERV-K in the subject. In some embodiments, an effective amount is an amount that reduces the level of at least one HERV-K protein in the subject. Sufficient amounts of a compound can also be referred to by the amount of the compound needed to inhibit growth of a specified amount of virus. Such an amount can be referred to as an inhibitory concentration (IC). For example, an IC50 refers to the concentration of drug necessary to inhibit 50% of viral growth. Likewise, IC90 refers to the concentration of drug necessary to inhibit 90% of viral growth.

As used herein, the term viral load refers to the amount of virus present in the subject. Viral load is typically determined by obtaining a sample from a subject, and determining the amount of virus in the sample. Methods of measuring reductions in viral load are known in the art. For example, such reductions can be measured as “fold reductions, percentages, and/or inhibition of growth. To illustrate fold reduction, a 2-fold (a factor of 2) reduction means a vial load that has been cut in half; a four-fold reduction means the viral load has been cut to one-fourth (reduced by a factor of four), etc.

As used herein, biological sample encompasses a variety of sample types obtained from a subject and can be used in a diagnostic or monitoring assay. The term biological sample encompasses blood, cerebral spinal fluid (CSF), urine and other liquid samples of biological origin, solid tissue samples, such as a biopsy specimen (e.g., muscle, brain, liver, etc.), or tissue culture cells or cells derived there from, and progeny thereof. The term also includes samples that have been manipulated in any way after their procurement, such as by treatment with reagents, solubilization, or enrichment for certain components, such as proteins or polynucleotides. The term biological sample encompasses a clinical sample, and also includes cells in culture, cell supernatants, cell lysates, serum, plasma, biological fluid, and tissue samples. Generally, the particular biological sample will depend on the type of probe target to which a detection assay is directed. For example, if the probe target is HERV-K RNA, the biological sample can be a blood sample, a CSF sample or a sample of neuronal tissue. A blood sample is a biological sample which is derived from blood, preferably peripheral (or circulating) blood. A blood sample may be, for example, whole blood, plasma or serum.

Biological samples can be used to determine the viral load of a subject by determining the amount of virus present in the sample. Any known method of detecting a virus and quantifying an amount thereof can be used to determine viral load. Such determination can be based on detecting viral proteins, viral RNA, viral DNA and/or whole virus particles using an appropriate assay (e.g., ELISA, nucleic acid hybridization assay, titration assay, etc.). Such determination can also be made by titering the amount of virus using tissue culture cells. In one embodiment, the amount of virus is measured in a blood sample. Methods of measuring an amount of HERV-K virus are known in the art. In one embodiment, the virus can be measured by detection of viral RNA. The subject may have greater than 100 copies of HERV-K gag RNA/ml of whole blood. The subject may also have greater than 1000 copies of HERV-K gag RNA/ml of whole blood.

As used herein, a human endogenous retrovirus (HERV) is a retrovirus that is present in the form of proviral DNA integrated into the genome of all normal cells and is transmitted by Mendelian inheritance patterns. Such proviruses are products of rare infection and integration events of the retrovirus into germ cells of the ancestors of the host. Most endogenous retroviruses are transcriptionally silent or defective, but can be activated under certain conditions. Expression of the HERV retrovirus may range from transcription of selected viral genes to production of complete viral particles, which may be infectious or non-infectious. Thus, in some cases, endogenous retroviruses may also be present as exogenous retroviruses. These variants are included in the term HERV for the purposes of the disclosure. In the context of the disclosure, human endogenous retrovirus includes proviral DNA corresponding to a full retrovirus comprising two LTRs, gag, pol, and env, and can further includes remnants of such a full retrovirus, which have arisen as a results of deletions in the retroviral DNA. Such remnants include fragments of the full retrovirus, and have a minimal size of one LTR. Typically, the HERVs have at least one LTR, preferably two, and all or part of gag, pol, and/or env proteins.

HERVS can be divided into different families based on the degree of nucleic acid similarity to other retroviruses, as well as other features such as the tRNA primer that is used in replicating the viral genome. For example, HERV-K uses a lysine tRNA as a primer for converting its viral RNA into a double-stranded DNA genome. As used herein, HERV-K refers to a retrovirus having a genome sufficiently identical to known HERV-K viruses that it would be recognized as an HERV-K retrovirus by one skilled in the art.

The methods of this disclosure are useful for treating ALS based on their ability to reduce the viral load of HERV-K virus, and the term reduced viral load is meant to be used in reference to the amount of virus observed in the absence of a particular compound.

The term compound, pharmaceutical compound, pharmaceutical agent, drug, and the like, can be used interchangeably herein, and include pharmacologically active substances in isolated form, or mixtures thereof. For example, a pharmaceutical agent, compound or drug may be an isolated and structurally-defined product, an isolated product of unknown structure, a mixture of several known and characterized products, or an undefined composition comprising one or more products. Examples of such undefined compositions include for instance tissue samples, biological fluids, cell supernatants, vegetal preparations, etc. The pharmaceutical agent, compound or drug may be any organic or inorganic product, including a polypeptide (or a protein or peptide), a nucleic acid, a lipid, a polysaccharide, a chemical entity, or mixture or derivatives thereof. The pharmaceutical agent, compound or drug may be of natural or synthetic origin, and the compound(s) or modulators may include libraries of compounds.

Anti-Retro Viral Drug Therapy

This disclosure provides methods of treating amyotrophic lateral sclerosis (ALS), or an ALS-like syndrome, in a subject diagnosed as having ALS, including administering to the subject one or more compounds that reduce the viral load of HERV-K retrovirus in the subject. Compounds effective in these methods may reduce the viral load by inhibiting one or more viral activities. As used herein, the term inhibit refers to the ability of a compound to reduce the level of activity of a viral protein to a level where the viral load is reduced. Such reduction can be either partial or complete. For example, a compound may inhibit a viral protein's activity by at least 10%, at least 20%, at least 50%, at least 75%, at least 80% or at least 95%, relative to the level of activity observed in the absence of compound. In a further example, a compound may reduce the level of a viral protein's activity to levels that are undetectable. Retroviruses are known to encode, at least, one reverse transcriptase, a protease, and an integrase. Consequently, any compound that inhibits the activity of such enzymes can be used in methods of the disclosure. Thus, these compounds may include at least one inhibitor selected from a reverse transcription inhibitor, a protease inhibitor and an integrase inhibitor. Reverse transcriptase, protease, and integrase inhibitors are known in the art, for example, in the field of anti-HIV therapy. Thus, compounds that act in a similar manner to those used in HVI-therapy, but which are particularly effective against HERV-K can be used in the present disclosure. Examples of such compounds include, but are not limited to, Abacavir, Zidovudine, Lamivudine, Stavudine, Tenofovir, Efavirenz, Etravirine, Nevirapine, Lopinavir, Tipranavir, Saquinavir, Nelfinavir, Amprenavir, Darunavir, Indinavir, and Atazanavir, and Raltegravir. Thus, one embodiment of this disclosure is a method of treating ALS in a subject diagnosed as having ALS, by administering to the subject one or more compounds selected from the group consisting of a reverse transcription inhibitor, a protease inhibitor and an integrase inhibitor. These compounds may include one or more of Abacavir, Zidovudine, Lamivudine, Stavudine, Tenofovir, Efavirenz, Etravirine, Nevirapine, Lopinavir, Tipranavir, Saquinavir, Nelfinavir, Amprenavir, Darunavir, Indinavir, and Atazanavir, and Raltegravir.

The inventors have discovered that certain drugs used in anti-retroviral therapy are only modestly effective or are completely ineffective in reducing HERV-K viral load when administered as a stand-alone therapy. Thus, the methods of anti-retroviral therapy may include more than one active ingredient, and a subject diagnosed as having ALS, may include the administration of two or more compounds selected from Abacavir, Zidovudine, Lamivudine, Stavudine, Tenofovir, Efavirenz, Etravirine, Nevirapine, Lopinavir, Tipranavir, Saquinavir, Nelfinavir, Amprenavir, Darunavir, Indinavir, and

Atazanavir, and Raltegravir. In one embodiment, the subject is administered a therapeutically effective amount of Darunavir, Ritonavir, Zidovudine and Raltegravir. A compound of the disclosure is any compound that can reduce the viral load of HERV-K retrovirus to a desirable and/or clinically effective level. A clinically effective level refers to a level at which at least some reduction in ALS-associated symptoms is achieved.

The compounds, or combinations of compounds, administered in these methods may reduce the viral load by at least 2-fold, by at least 4-fold, by at least 5-fold, by at least 10-fold, by at least 25-fold, by at least 50-fold, by at least 100-fold, by at least 500 fold, by at least 1000-fold, by at least 10,000-fold, or by at least 100,000-fold. Similarly, the compounds, or combinations of compounds, administered may reduce the viral load by at least 10¹, at least 10², at least 10³, at least 10⁴, at least 10⁵, at least 10⁶ or at least 10⁷. Similarly, the compounds, or combinations of compounds, administered may reduce the viral load by at least 5%, at least 10%, at least 20%, at least 30%, at least 40%, at least 50%, at least 60%, at least 70%, at least 80%, at least 90%, at least 95%, or at least 99%.

The compounds, or combinations of compounds, administered may inhibit HERV-K replication by at least 5%, at least 10%, at least 20%, at least 30%, at least 40%, at least 50%, at least 60%, at least 70%, at least 80%, at least 90%, at least 95%, or at least 99%. These compounds, or combinations of compounds, administered may have an IC90 of less than 1 μM, or less than 0.8 μM, or less than 0.5 μM, or less than 0.1 μM, with respect to the retroviral enzyme they are designed to inhibit.

These compounds, or combinations of compounds, administered may reduce the viral load to undetectable levels in a biological sample from the subject. This may include reducing the viral load to undetectable levels in the subject's blood.

In addition to the retroviral enzyme inhibitors described above, compounds useful for practicing the methods of the present disclosure are those that interfere with, or inhibit, transcription of the viral genome, and/or translation of viral transcripts. As used herein, and in the context of transcription and translation, the term inhibit refers to the ability of a compound of the invention reduce the amount of mRNA transcripts, or proteins encoded by such transcripts. Such reduction may be partially or it may be total. For example, a compound of the invention may reduce the level of HERV-K transcripts, or proteins encoded by such transcripts, by at least 10%, at least 20, at least 50%, at least 75%, at least 90% or at least 95%, relative to the level of transcripts, or proteins, observed in the absence of the compound. As another example, a compound may reduce the level of transcripts, or proteins encoded by such transcripts, or proteins encoded by such transcripts, to undetectable levels. Such inhibition can be achieved, for example, by using one or more compounds that bind components of the HERV-K transcription system and/or components of the HERV-K translation system. Such components include, but are not limited to, HERV-K nucleic acid molecules, such as the HERV-K genome, or HERV-K mRNA, HERV-K encoded proteins, and proteins made by the subject's cells. More specific examples of such proteins include, but are not limited to, HERV-K polymerases, cellular polymerases, transcription factors and repressors. The compounds may bind to at least one component of the HERV-K transcription system. The compounds may inhibit transcription from HERV-K nucleic acid sequences. The compounds may bind to at least one component of the HERV-K transcription system, wherein binding of the transcription component results in inhibition of transcription. The compounds may bind to the HERV-K genome, thereby inhibiting transcription of HERV-K nucleic acid sequences. The compounds may bind components of the HERV-K transcription or translation system at specific binding sites. As used herein, a binding site refers to a polynucleotide sequence recognized by a component of the HERV-K transcription or translation system, and which is necessary for binding of the component to a HERV-K nucleic acid molecule. For example, a HERV-K RNA polymerase can bind the HERV-K genome by recognizing an RNA polymerase binding site and subsequently binding to the genome. The components may bind the HERV-K nucleic acid molecule at the binding site or may bind the HERV-K nucleic acid molecule at a site near, but distinct from, the binding site. That is, portions (e.g., amino acids, nucleotides) of the component may contact nucleotides in the binding site or may contact nucleotides outside of the binding site. Portions of components may also contact nucleotide residues both within and outside the binding site.

The compounds useful in the methods of the present disclosure can bind at any location or sequence in the HERV-K genome, as long as such binding results in inhibition of transcription of HERV-K sequences. Such sites include, but are not limited to, polymerase binding sites (e.g., promoter sequences), operator site (i.e., repressor protein binding sites), enhancer protein binding sites, and transcription factor binding sites. Thus, the compound(s) may bind to the HERV-K genome in a promoter region. The compound(s) may bind to the HERV-K genome at a polymerase binding site, thereby inhibiting binding of a polymerase to the binding site. The compound(s) may bind to the HERV-K genome at location near a transcription factor binding site, wherein such binding inhibit binding of the transcription factor to the transcription factor binding site. The compound(s) may bind to the HERV-K genome at a transcription factor binding site, thereby inhibiting binding of a transcription factor to the transcription factor binding site.

The LTR region of the HERV-K genome has promoter function and contains binding sites for proteins that affect transcription of HERV-K sequences. Thus, the compound(s) may bind a polynucleotide sequence in the LTR of the HERV-K genome, wherein such binding affects transcription of HERV-K nucleic acid sequences. The compound(s) may bind a polynucleotide sequence in the LTR of the HERV-K genome, wherein such binding inhibits transcription of HERV-K nucleic acid sequences. The compound(s) may bind a promoter region in the LTR of the HERV-K genome. The compound(s) may bind to a polymerase binding site in the LTR of the HERV-K genome. The compound(s) may bind a location near a transcription factor binding site in the LTR of the HERV-K genome. The compound(s) may bind a transcription factor binding site in the LTR of the HERV-K genome.

HERV-K pol gene expression has been found to correlate with TAR DNA-binding protein 43 (TDP-43) mRNA in post-mortem brain tissue from patients with ALS (Douville and Nath, supra). TDP-43 is an RNA binding protein containing two RNA-recognition motifs (RRM), a nuclear localization signal (NLS), a nuclear export signal (NES), as well as a C-terminal glycine-rich domain (GRD) implicated in TDP-43 protein interactions and functions. The protein is normally concentrated in the nucleus but also shuttles back and forth between the nucleus and cytoplasm. TDP-43 aggregation and neuropathology have been found to play a role in a broad spectrum of neurodegenerative disorders. (Cohen et al., 2011, Trends Mol. Med. 17, 659-667; Buratti et al., 2012, RNA Biol. 7, 420-429; Sendtner et al., 2011, Nat. Neurosci. 14, 403-405). Cytosolic accumulation of truncated TDP-43 is found in affected neurons of patients suffering from sporadic and familial ALS and FTLD. (Cohen et al., 2011, Trends Mol. Med. 17, 659-667; Lander et al., 2001, Nature 409, 860-921; Hua-Van et al., 2011, Biol. Dir. 6, 19.) Missense mutations clustering in the TDP-43 GRD have been identified in cases of ALS (Hancks and Kazazian, 2012, 22, 191-202; Saito and Siomi, 2010, Dev. Cell. 19, 687-697). TDP-43 has also been shown to regulate the replication of human immunodeficiency virus (HIV) (Ou et al., J. Virol 69, 3584-3595 (1995)) and to also bind to transposable elements (Li. et al., PloS One 7, e44099 (2012)). Thus, compounds useful in the methods of this disclosure may bind a site near a TDP-43 site, such that binding of TDP-43 to the TDP-43 binding site is inhibited. Thus, one embodiment of the present invention is a method for treating ALS, comprising administering to a subject diagnosed as having ALS, a compound that binds the HERV-K genome near, or at, the TDP-43 binding site such that binding of TDP-43 to the TDP-42 binding site is inhibited. The compound(s) may bind a site near a TDP-43 site, such that binding of TDP-43 to the TDP-43 binding site is blocked. The compound(s) may bind to a TDP-43 binding site. The compound(s) may bind to a TDP-43 binding site in the LTR of the HERV-K genome. Binding of the compound(s) to the TDP-43 binding site may inhibit transcription of HERV-K nucleic acid sequences.

The TDP-43 binding site is known to contain pyrimidine-rich motifs associated with TDP-43 DNA binding. Thus, the compounds useful in the methods of this disclosure may bind to a polynucleotide sequence containing eight or more contiguous pyrimidine bases. These compound(s) may bind to a polynucleotide sequence in the LTR of the HERV-K genome, wherein the polynucleotide sequence contains eight or more contiguous pyrimidine bases. The compound(s) may bind to a sequence in the HERV-K genome comprising at least one sequence selected from the group consisting of SEQ ID NO:1 (TTTCTCCCC), SEQ ID NO:2 (CCCTCTCCC), SEQ ID NO:3 (CCCCCTCTTT), SEQ ID NO:4 (TTTCTTTCTCT), and SEQ ID NO:5 (TCTTTTTCTTTTCC). The compounds may comprise a sequence selected from the group consisting of SEQ ID NO:6 (GGGGAGAAA), SEQ ID NO:7 (GGGAGAGGG), SEQ ID NO:8 (AAAGAGGGGG), SEQ ID NO:9 (AGAGAAAGAAA) and SEQ ID NO:10 (GGAAAAGAAAAAGA)

Inhibition of TDP-43 activity can also be inhibited by direct binding of the TDP-43 protein. Thus, the compounds useful in the methods of this disclosure may bind to the TDP-43 protein, thereby inhibiting binding of the TDP-43 protein to the TDP-43 binding site.

While the expression of HERV-K proteins can be inhibited by inhibiting transcription, the production of such proteins can also be blocked by inhibiting translation of HERV-K mRNA molecules. Thus, the compounds useful in the methods of this disclosure may inhibit translation of HERV-K mRNA. These compound(s) may bind to at least one component of the HERV-K translation system. These compound(s) may bind HERV-K mRNA. These compound(s) may bind to at least one component of the HERV-K translation system, wherein binding of the translation system component results in inhibition of translation of HERV-K mRNA. Binding of these compound(s) to a HERV-K mRNA molecule may increase degradation of the bound molecule.

As noted above, compounds useful in the methods of this disclosure may be any organic or inorganic product, including polypeptides, nucleic acid molecules, lipids, polysaccharides, chemical entities (e.g., small organic molecules), or mixture or derivatives thereof. A particularly useful compound is a therapeutic oligonucleotide (tON). Thus, this disclosure provides methods of treating or preventing ALS in a subject by administering to a subject diagnosed with ALS, or a subject at risk for developing ALS, a compound comprising a tON, wherein the tON is capable of reducing the viral load of HERV-K in the subject. As used herein, a therapeutic tON is a synthetic nucleic acid molecule that reduces the viral load of HERV-K in a subject or reduces the level of one or more HERV-K proteins in a subject. tONs of this disclosure may act by one of two mechanisms. tONs may bind to the HERV-K genome, thereby inhibiting transcription of a HERV-K mRNA. Such inhibition can be due to the presence of the tON physically blocking (steric hindrance) a transcription system component (e.g., a polymerase, a transcription factor, etc.) from binding to its binding site in the HERV-K genome. Alternatively, a tON binding to the HERV-K can inhibit transcription by interfering with elongation of transcript by a polymerase. Binding of a tON to the HERV-K genome may thereby result in alteration of the HERV-K genome, such that transcription of HERV-K nucleic acid sequences cannot occur. For example, the tON can have catalytic activity that cleaves the HERV-K genome or otherwise alters the HERV-K genome. A tON of the disclosure can be composed of RNA or DNA. The tON may be selected from an antisense RNA, an inhibitory RNA, a small hairpin RNA (shRNA), a micro RNA (miRNA) and an aptamer. General methods for designing and making such molecules are known in the art and are described in U.S. Patent Publication Nos. 2014/0099666, 2014/0113952, and 20140377758, which are incorporated herein by reference in their entirety.

Methods of treating ALS in a subject may therefore include administering to a subject diagnosed with ALS, or an ALS-like syndrome, a compound comprising a tON, wherein the tON is capable of reducing the viral load of HERV-K in the subject. The tON may be capable of reducing the viral load of HERV-K in the subject. The tON may bind to the HERV-K genome and prevent binding of a polymerase or a transcription factor to a binding site in the HERV-K genome. The tON may bind to a polymerase binding site in the HERV-K genome. The tON may bind to a transcription factor binding site in the HERV-K genome. The tON may bind to a TDP-43 binding site in the HERV-K genome. The tON may bind to a polynucleotide sequence in the LTR of the HERV-K genome, wherein the polynucleotide sequence contains eight or more contiguous pyrimidine bases in. The tON may bind to a sequence in the HERV-K genome comprising at least one sequence selected from the group consisting of SEQ ID NO:1, SEQ ID NO:2, SEQ ID NO:3, SEQ ID NO:4, and SEQ ID NO:5. The tON may comprise a sequence selected from the group consisting of SEQ ID NO:6, SEQ ID NO:7, SEQ ID NO:8, SEQ ID NO:9, and SEQ ID NO:10. The tON may bind to a HERV-K nucleic acid sequence encoding a HERV-K protein selected from an envelope protein, a polymerase protein, and a protease protein.

Alternatively, the tON may bind to an HERV-K mRNA molecule, and the binding of the tON to HERV-K may prevent translation of the mRNA. The tON may bind to a ribosomal binding site in the HERV-K genome. The tON may bind to a coding region of the mRNA. The tON may bind to one or more regions encoding a HERV-K protein selected from an envelope protein, a polymerase protein, and a protease protein.

The therapeutic oligonucleotides of the disclosure may also be modified to have beneficial properties. For example, tONs of this disclosure can be chemically modified to improve their resistance to ribonucleases, increase solubility and/or reduce immunogenicity. Such modifications can include, but are not limited to, changes in the sugar, base or backbone of the nucleic acid. Methods of modifying nucleic acid molecules to obtain improved properties are known in the art and are also disclosed in Burnett and Rossi, Chem Biol. 2012 Jan. 27; 19(1):60-71.

This disclosure therefore includes therapeutic oligonucleotides (tON) useful for treating ALS in a subject diagnosed as having ALS, whereby administration of the tON to the subject results in a reduction in the viral load of HERV-K retrovirus. These therapeutic oligonucleotides may also be useful for preventing ALS in a subject at risk for developing ALS, whereby administration of the tON to the subject results in prevention of the onset of ALS in a subject suspected of having, or susceptible to developing, ALS. The tON may bind to the HERV-K genome and prevent binding of a polymerase or a transcript factor to a binding site in the HERV-K genome. The tON may bind to a polymerase binding site in the HERV-K genome. The tON may bind to a transcription factor binding site in the HERV-K genome. The tON may bind to a TDP-43 binding site in the HERV-K genome. The tON may bind to a polynucleotide sequence in the LTR of the HERV-K genome, wherein the polynucleotide sequence contains eight or more contiguous pyrimidine bases in. The tON may bind to a sequence in the HERV-K genome comprising at least one sequence selected from the group consisting of SEQ ID NO:1, SEQ ID NO:2, SEQ ID NO:3, SEQ ID NO:4, and SEQ ID NO:5. The tON may comprise a sequence selected from the group consisting of SEQ ID NO:6, SEQ ID NO:7, SEQ ID NO:8, SEQ ID NO:9, and SEQ ID NO:10. The tON may bind to a HERV-K nucleic acid sequence encoding a HERV-K protein selected from an envelope protein, a polymerase protein and a protease protein. The tON may bind to a HERV-K mRNA molecule. The binding of the tON to a HERV-K mRNA may prevent translation of the mRNA. The tON may bind to ribosomal binding site in the HERV-K genome. The tON may bind to a coding region of the mRNA. The tON may bind to a region encoding an HERV-K protein selected from an envelope protein, a polymerase protein, and a protease protein.

Any method of delivering tONs of this disclosure can be used, as long as the tON reaches its intended target. A tON may be delivered as naked nucleic acid molecule, or it may be encapsulated in a second molecule. For example, tONs may be encapsulated in DNA molecules or in lipid molecules. In one embodiment, a tON of the disclosure is packaged into a viral vector. Thus, this disclosure provides methods of treating ALS in a subject comprising administering to a subject diagnosed with ALS, or an ALS-like syndrome, a viral vector comprising, or expressing, a tON, wherein the tON is capable of reducing the viral load of HERV-K in the subject. The disclosure therefore includes methods of treating ALS in a subject diagnosed as having ALS, or an ALS-like syndrome, comprising administering to the subject a viral vector comprising, or expressing, a tON, wherein the tON is capable of reducing the viral load of HERV-K in the subject. This disclosure also provides methods of preventing ALS in a subject comprising administering to a subject at risk for ALS, or suspected of having ALS, a viral vector comprising, or expressing, a tON, wherein the tON is capable of reducing, or preventing an increase in, the viral load of HERV-K in the subject.

As used herein, a viral vector is a recombinant virus, or viral-like particle, comprising a nucleic acid sequence encoding a tON of the disclosure. The viral vector may contain tONs packaged into a viral particle. In one embodiment, the viral vector comprise gene expressing the tON. Any viral vector can be used to deliver a tON of the disclosure to subject. Examples of useful viral vectors include, but are not limited to, adenovirus vectors, adeno-associated virus vectors, lentivirus vectors herpes simplex virus (HSV) vectors and poxvirus vectors. General methods of preparing a viral vector for use in the present disclosure are known in the art and examples of such vectors are disclosed in U.S. Pat. No. 7,479,554, U.S. Pat. No. 7,718,424, U.S. Pat. No. 8,137,960, U.S. Pat. No. 8,283,151, U.S. Pat. No. 8,927,269, U.S. Pat. No. 9,133,478, and U.S. Pat. No. 9,133,480, all of which are incorporated herein by reference in their entirety. Thus, this disclosure further includes viral vectors comprising a tON, or comprising a nucleic acid sequence encoding a tON of this disclosure.

While methods of the present disclosure can be used to treat ALS, they can also be used to prevent ALS in individuals at risk for developing ALS. As used herein, the term “preventing ALS” refers to an approach for treating an individual, the outcome of which is that the individual does not develop ALS-associated symptoms or physical characteristics. The term preventing can be applied to a normal subject or a subject at risk for developing ALS. As noted above, several genetic polymorphisms have been associated with the development of ALS. Accordingly, as used herein, a subject at risk for developing ALS is an individual having a familial or physical link to ALS. For example, a subject having a genetically-linked family member (e.g., parent, child, sister, brother, etc.) that has developed ALS, is considered at risk for developing ALS. Similarly, a subject having a genetic marker known to be associated with the development of ALS is considered at risk for developing ALS. Thus, this disclosure encompasses methods of preventing the development of amyotrophic lateral sclerosis (ALS) in a subject, comprising administering to a subject at risk for developing ALS, one or more compounds sufficient to reduce the viral load of HERV-K retrovirus.

The inventors have also discovered that, surprisingly, the envelope protein (Env) of HERV-K has the ability to cause ALS-like symptoms. Specifically, when Env was expressed in neuronal cells, neurotoxicity was observed, as evidenced by retraction of neuritis and loss of neurons. Further, transgenic animals engineered to express Env in their neurons showed loss of upper and lower neurons, and development of motor dysfunction. Accordingly, this disclosure provides methods of treating or preventing ALS in an individual includes reducing the level of HERV-K Env protein in the individual. These methods may include administering to the subject one or more compounds sufficient to reduce the level of Env protein in the subject.

Such compounds may reduce the level of Env protein by at least 2-fold, by at least 4-fold, by at least 5-fold, by at least 10-fold, by at least 25-fold, by at least 50-fold, by at least 100-fold, by at least 500 fold, by at least 1000-fold, by at least 10,000-fold, or by at least 100,000-fold. Such compounds may reduce the level of Env protein by at least 10¹, at least 10², at least 10³, at least 10⁴, at least 10⁵, at least 10⁶ or at least 10⁷. Such compounds may reduce the level of Env protein by at least 5%, at least 10%, at least 20%, at least 30%, at least 40%, at least 50%, at least 60%, at least 70%, at least 80%, at least 90%, at least 95%, or at least 99%. Such compounds may inhibit HERV-K replication by at least 5%, at least 10%, at least 20%, at least 30%, at least 40%, at least 50%, at least 60%, at least 70%, at least 80%, at least 90%, at least 95%, or at least 99%. Such compounds may have an IC90 of less than 1 μM, or less than 0.8 μM, or less than 0.5 μM, or less than 0.1 μM. Such compounds may reduce the level of Env protein to undetectable levels in a biological sample from the subject. Such compounds may even reduce the viral load to undetectable levels in the subject's blood. Such compounds may be selected from Abacavir, Zidovudine, Lamivudine, Stavudine, Tenofovir, Efavirenz, Etravirine, Nevirapine, Lopinavir, Tipranavir, Saquinavir, Nelfinavir, Amprenavir, Darunavir, Indinavir, and Atazanavir, and Raltegravir. Such compounds may include the combination of Darunavir, Ritonavir, Zidovudine and Raltegravir. In specific methods, the compounds may consist of the combination of Darunavir, Ritonavir, Zidovudine and Raltegravir. In other methods, the compounds comprise a tON of this disclosure. In these methods, the tON may bind to the HERV-K genome and prevents binding of a polymerase or a transcription factor to a binding site in the HERV-K genome. The tON may bind to a polymerase binding site in the HERV-K genome. The tON may bind to a transcription factor binding site in the HERV-K genome. The tON may bind to a TDP-43 binding site in the HERV-K genome. The tON may bind to a polynucleotide sequence in the LTR of the HERV-K genome, wherein the polynucleotide sequence contains eight or more contiguous pyrimidine bases in. The tON may bind to a sequence in the HERV-K genome comprising at least one sequence selected from the group consisting of SEQ ID NO:1, SEQ ID NO:2, SEQ ID NO:3, SEQ ID NO:4, and SEQ ID NO:5. The tON may comprise a sequence selected from the group consisting of SEQ ID NO:6, SEQ ID NO:7, SEQ ID NO:8, SEQ ID NO:9, and SEQ ID NO:10. The tON may bind to a HERV-K nucleic acid sequence encoding the HERV-K Env protein. The tON may bind to a HERV-K mRNA molecule, which may prevent translation of the mRNA. The tON may bind to ribosomal binding site in the HERV-K genome. The tON may bind to a coding region of the mRNA. The tON may bind to a region of the mRNA encoding the HERV-K Env protein.

The tON useful in these methods of reducing the level of Env transcript and/or protein in the subject may be administered in the form of a naked nucleic acid molecule. The tON may be encapsulated in one or more additional molecules. The additional molecules may be lipid molecules and/or nucleic acid molecules. The tON may be administered in the form of a viral vector comprising the tON.

### Transgenic Animals

Because transgenic animals expressing HERV-K protein in their neurons display symptoms associated with ALS, such animals are useful for studying the disease and testing compounds for their ability to prevent the development of ALS or treat ALS. Thus, this disclosure encompasses transgenic animals expressing the HERV-K Env protein in its neuronal cells. In one embodiment, the animal is a mouse, rat, rabbit, dog or non-human primate. General methods of making transgenic animals are known in the art and are described in U.S. Patent Publication Nos. 2003/0167489, 2003/0110522, and 2006/0135612, the disclosures of which are incorporated herein by reference in their entirety.

### Reporter Cells

The methods and tools disclosed herein are also useful for identifying compounds useful for treating or preventing ALS. For example, using the present disclosure, a reporter cell can be constructed in which a gene encoding a reporter protein is placed under the control of HERV-K promoter sequences such that in the absence of any inhibitory compounds, the reporter protein is produced and the cell emits a detectable signal. Examples of such reporter protein include, but are not limited to, firefly luciferase protein and green fluorescent protein. Other such reporter proteins are known to those skilled in the art. When a compound that is capable of inhibiting transcription of HERV-K promoters is added to the reporter cell, no transcription of the reporter gene occurs and thus, no signal is produced. Thus, one embodiment of the present disclosure is an assay system comprising a recombinant cell, wherein the recombinant cell comprises a nucleic acid molecule comprising 1) a nucleic acid sequence encoding a reporter protein (i.e., a reporter gene); and, 2) one or more nucleic acid sequence from the HER-K genome, wherein the one or more HERV-K nucleic acid sequences comprise binding sites for components of the HERV-K transcription system, wherein the reporter gene is functionally linked to the HERV-K nucleic acid sequences such that the HERV-K nucleic acid sequence can regulate expression of the reporter gene. As used herein, the term “functionally linked” refers to two or more nucleic acids sequences, or partial sequences, which are positioned so that they functionally interact to perform their intended functions. For example, a promoter is functionally linked to a nucleic acid (e.g., coding) sequence if it is able to control or modulate transcription of the linked nucleic acid sequence in the cis position. Generally, but not necessarily, functionally linked nucleic acid sequences are close together. Although a functionally linked promoter is generally located upstream of the coding sequence it does not necessarily have to be close to it. Enhancers need not be close by, provided that they assist the transcription of the nucleic acid sequence. For this purpose, enhancers may be both upstream and/or downstream of the nucleic acid sequence, possibly at some distance from it. A polyadenylation site is functionally linked to a gene sequence if it is positioned at the 3′ end of the gene sequence in such a way that the transcription progresses via the coding sequence to the polyadenylation signal. Accordingly, two or more nucleic acid sequences that are functionally linked may or may not be in direct contact (i.e., immediately adjacent to one another in the virus vector genome). The HERV-K nucleic acid sequences may be from a HERV-K promoter region. The HERV-K nucleic acid sequences may be from a HERV-K LTR region. The HERV-K nucleic acid sequences may comprise a HERV-K protein binding site. The HERV-K nucleic acid sequences may comprise a polymerase binding site. The HERV-K nucleic acid sequences may comprise a binding site for a transcription factor. The HERV-K nucleic acid sequences may comprise a TDP-43 binding site. The HERV-K nucleic acid sequences may comprise a polynucleotide sequence containing eight or more contiguous pyrimidine bases. The HERV-K nucleic acid sequences may comprise a polynucleotide sequence comprising at least one sequence selected from the group consisting of SEQ ID NO:1, SEQ ID NO:2, SEQ ID NO:3, SEQ ID NO:4, and SEQ ID NO:5.

### Kits

This disclosure also includes kits suitable for practicing methods of the present disclosure. Kits can include, for example, compounds of the disclosure, recombinant virus vectors of this disclosure, nucleic acid molecules for constructing recombinant virus vectors of this disclosure, and/or recombinant cells for practicing methods of the disclosure. Kits may also comprise associated components, such as, but not limited to, proteins, enzymes, cell culture media, buffers, labels, containers, vials, syringes, instructions for using the kit and the like.

### EXAMPLES

These examples demonstrate that HERV-K is activated in a subpopulation of patients with sporadic amyotrophic lateral sclerosis (ALS) and that its envelope (env) protein may contribute to neurodegeneration. The inventors began by designing studies to determine if HERV-K could play a role in the pathogenesis of amyotrophic lateral sclerosis. They first showed the expression of each of the transcripts of HERV-K in the brains of 11 patients with ALS. Brain tissues from 16 individuals with no known brain disease were used as controls. Samples were matched for sex, postmortem interval, RNA integrity values and the anatomical region of the brain studied. Immunohistochemistry showed that expression of the virus was localized to cortical neurons in brain tissue and anterior horn cells in spinal cord obtained from 10 patients with ALS. Brain tissues from 10 patients with Alzheimer's disease were used as controls. Sample sizes were based on availability of tissues and prior experience with such assays. To determine if the expression of the virus in neurons could cause neurotoxicity, the inventors transfected neuronal cultures with plasmids containing entire HERV-K, HERV-K env or pcDNA and monitored for cell counts or change in neurite length. All experiments were done in replicates of 18, and repeated twice. Similar amounts of toxicity were seen with HERV-K and HERV-K env. Hence, in a pilot experiment, the inventors injected the HERV-K env plasmid (n=11) or control plasmid (n=9) into the mouse brain in utero. Expression of HERV-K env reproduced the morphological abnormalities in the neurons. We next generated transgenic animals in which the HERV-K env was expressed under a neuronal promoter. Non-transgenic littermates were used controls. The transgenic animals developed progressive motor dysfunction over 6 months at which time nearly 50% of the animals died. Due to profound motor abnormalities in the animals, it was not possible to perform the testing in a blinded manner. But all histopathological and radiological assessments were performed by an investigator blinded to the genotype of the animals. The transgenic animals had selective thinning of the motor cortex, morphological abnormalities in neurites of the motor neurons with DNA strand breaks and nucleolar abnormalities. There was accompanying astrocytosis. All behavioral experiments were done in sample sizes of at least 15 animals in each group and histological studies had at least 5 animals in each group. The regulation of HERV-K expression was studied in vitro. These data were analyzed from at least three separate experiments. It showed that TDP-43 binds to the LTR of HERV-K to regulate its expression.

### Cell Culture and Transfection

The human cell lines 293T and Hela were maintained in DMEM supplemented with 10% FBS and penicillin-streptomycin. For testing activity of HIV-RT inhibitors in a cell free system, Hela cells were transiently transfected with pCD-HK/Rev in 24-well plates at 0.2×106 cells/well using lipofectamine 2000 (Invitrogen) according to the manufacture's protocol. Virus particle-containing supernatants were collected after 24 and 48 hrs. Control experiments included mock transfection with empty vector pcDNA3.1. Cell culture supernatants were assayed for RT activity using a PERT assay as described below. At the time of reverse transcription, HIV nucleoside or non-nucleotide RT Inhibitors were added to the supernatant at six different doses ranging from 0.001 μM to 0.25 μM. Any change in RT activity was expressed as percent inhibition relative to no treatment control.

For testing the activity of HIV protease inhibitors against HERV-K, HeLa cells were transiently transfected with pCD-HK/Rev as described above. Six hrs post-transfection, culture medium was completely replaced with fresh medium containing HIV protease inhibitors in a 2-fold serial dilution ranging from 31.25 nM to 1 μM. After 48 hrs, cell culture supernatants were collected and RT activity in the culture supernatant was determined by PERT assay. Darunavir and Lopinavir were identified as the two most potent drugs and were further screened in a 10 fold-serial dilution treatment ranging from 0.001 μM-100 μM.

### Quantitative PCR.

All human samples were obtained following approval by the Office of Human Subjects Research Protection at NIH and the Institutional Review Boards at Johns Hopkins University. All samples were analyzed by investigators blinded to the clinical condition or identity of the patients. Total RNA was extracted from frozen brain tissue with RNeasy Plus mini kit (Qiagen, Alameda, Calif.). The RNA extracts were treated with RNase-free DNase (Qiagen). The quality of RNA was evaluated with Agilent 2100 Bioanalyzer. Only samples which had a RNA integrity number (RIN) of greater than 8 were used for this study to ensure that there was little or no RNA degradation in the samples. Reverse transcription was performed with 1 pg RNA using Superscript III first stand kit (Invitrogen). Quantitative PCR was done using Applied Biosystems ViiA 7 (Grand Island, N.Y.). The amount of RNA in brain samples was expressed as relative levels to control samples after normalization with GAPDH RNA. To confirm that there was no DNA contamination; control PCR reactions were performed with reverse transcription product in which Superscript III was omitted. Primers for quantitative PCR are shows in the following table:

Primers for qualitative PCR

| | | |
|---|---|---|
| HERV-K env | Forward: | CTGAGGCAATTGCAGGAGTT (SEQ ID NO: 17) |
| | Reverse: | GCTGTCTCTTCGGAGCTGTT (SEQ ID NO: 18) |
| HERV-K pol | Forward: | TCACATGGAAACAGGCAAAA (SEQ ID NO: 19) |
| | Reverse: | AGGTACATGCGTGACATCCA (SEQ ID NO: 20) |
| HERV-K gag | Forward: | AGCAGGTCAGGTGCCTGTAACATT (SEQ ID NO: 21) |
| | Reverse: | TGGTGCCGTAGGATTAAGTCTCCT (SEQ ID NO: 22) |
| HERV-K env (47) | Forward: | GGGCCAATTATGCTTACCAA (SEQ ID NO: 23) |
| | Reverse: | ATGGGCTGATCTGGCTCTAA (SEQ ID NO: 24) |
| HERV-K env (48) | Forward: | CTGGTCCACGCACGGCCGAAGCATG (SEQ ID NO: 25) |
| | Reverse: | AAAAGGACGACTTAATAGAGCCAAT (SEQ ID NO: 26) |
| HERV-K env (49) | Forward: | CAAGATTGGGTCCCCTCAC (SEQ ID NO: 27) |
| | Reverse: | CCTATGGGGTCTTTCCCTC (SEQ ID NO: 28) |
| GAPDH | Forward: | TGCACCACCAACTGCTTAGC (SEQ ID NO: 29) |
| | Reverse: | GGCATGGACTGTGGTCATGAG (SEQ ID NO: 30) |
| tg-HERV-K env | Forward: | GTGTGCCTGTTTTGTCTGC (SEQ ID NO: 31) |
| | Reverse: | CACGATCTGGTCCCTTTTACTC (SEQ ID NO: 32) |
| mGAPDH | Forward: | AGGTCGGTGTGAACGGATTTG (SEQ ID NO: 33) |
| | Reverse: | GGGGTCGTTGATGGCAACA (SEQ ID NO: 34) |
| | Forward: | TCCTGCTCAACTTCCTGTCGA (SEQ ID NO: 35) |
| | Reverse: | CACAGGTCAAACCTCCTAGGAATG (SEQ ID NO: 36) |
| | Probe: | CGAGACGCTACCATGGCTATCGCTGTAG (SEQ ID NO: 37) |

The HERV-K full length primer sequences were designed to amplify the unspliced full length HERV-K transcript without the LTRs and the spliced transcripts representing env and rec. Primers for transgenic-HERV-K env and mGAPDH were used to quantify relative expression level of env in the transgenic mice.

### DNA Constructs and HIV Inhibitors

HERV-K whole genome consensus sequence was synthesized and cloned into pcDNA3.1 vector (Invitrogen). HIV-1 Rev plasmid was reported previously. To increase the production of HERV-K viral particles, the Rev expression cassette was inserted to the pcDNA3.1-HERV-K construct. The resulting plasmid was called pCD-HK/Rev. All HIV inhibitors and VSV-G plasmid were obtained from NIH AIDS reagent program (http://www.aidsreagent.org). A stock of 10 mM was made by diluting the inhibitors in dimethyl sulfoxide (DMSO). For further use serial dilutions for each inhibitor was made in complete media: Dulbecco's modified Eagle's medium; DMEM+10% fetal bovine serum (FBS) and penicillin-streptomycin.

### Induction of Endogenous HERV-K by Lentiviral Transduction.

Lentiviral constructs encoding HERV-K LTR-targeting guide RNA (sgRNA) or nuclease-null Cas9 linked to transcription activator domain VP64 were generated using the Virapower Lentiviral Packaging Mix according to the manufacturer's protocol (Invitrogen; Grand Island, N.Y.). Lentivirus stocks obtained from transfection of HEK293T cells were concentrated using Retro-X Concentrator (Clontech; Mountain View, Calif.) and copy number was determined by Lenti-X qRT-PCR Titration kit (Clontech). dCas9-NLS-3×HA-VP64 vector (Addgene) served as source of insert material for the Cas9 lentivirus. Briefly, neural stem cell (NSC)-derived neurons were incubated with Cas9-VP64 lentiviral vector in presence of 5 pg/ml polybrene (Sigma; St. Louis, Mo.). After 4 hours, inoculum was diluted with an equal amount of complete medium and cells were incubated overnight. 24 hours post-transduction, supernatant was removed and cells were incubated with lentiviral vector expressing sgRNA targeting the HERV-K promoter (sgRNA 8) in presence of 5 pg/ml polybrene for an additional 24 hours.

### Immunohistochemistry of Human Autopsy Brain Tissue.

The inventors analyzed human autopsy tissues of ten patients with ALS from the ALS Center at the university in Pittsburgh. Samples from the frontal cortex and the cervical spinal cord were examined. The mean age of patients at the time of death was 59.3 years (range 45-73 years) and patients were 50% male and 50% female. The autopsy was done an average of 7.1 hours after death (range 2-10 hours). Sections from ten Alzheimer's patients served as controls. We obtained Alzheimer's patients samples from the Department of Pathology at the University of Kentucky. The mean age of patients at the time of death was 84.5 years (range 74-95 years) and patients were 50% male and 50% female. The autopsy was done an average of 3.4 hours after death (range 2-5 hours). Autopsied brains were fixed in formalin and paraffin-embedded. Five micron thick sections were obtained and stained as follows. The slides were deparafinized and re-hydrated using Xylene and graded ethanol. Antigen retrieval was done by steaming in citrate buffer for 20 minutes. Peroxidase blocking was achieved with dual enzyme block (DAKO, Carpinteria, Calif.) and protein blocking was done with protein block solution (DAKO). Incubation with mouse anti-HERV-K env (1:500; Austral Biologicals, San Ramon, Calif.) or anti-beta amyloid (1:500; BioLegend, San Diego, Calif.) was done for overnight at room temperature. Powervision poly-HRP mouse (Leica Biosystems, Buffalo Grove, Ill.) or HRP-conjugated anti-human IgG was applied as secondary antibodies for two hours at room temperature. Antibody binding was developed with 3,3′-diaminobenzidine (DAB; Vector Laboratories, Burlingame, Calif.). Sections were counterstained with hematoxylin (Dako). Images were processed using a whole slide scanner, Aperio (Leica Biosystems).

### Generation of Transgenic Mice.

The Thy1-HERV-K env transgene cassette was excised with EcoRI and PvuI. The purified fragment was injected into the pronuclei of fertilized eggs from C57BL6 mice. Surviving embryos were implanted into pseudo-pregnant C57BL6 mice. The transgenic founders were screened by genotyping with primer set

| (SEQ ID NO: 38) | |
|---|---|
| GT-env-F2: | 5′-ACCAGCTGGCTGACCTGTAG-3′ |
| and | |
| (SEQ ID NO: 39) | |
| GT-env-R2: | 5′-GGCAGCTTCATCTGTTCCTC-3′ |

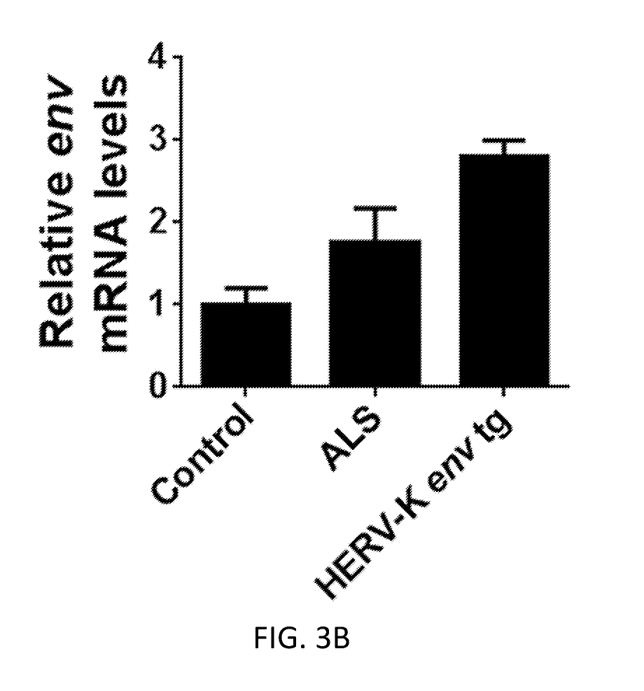

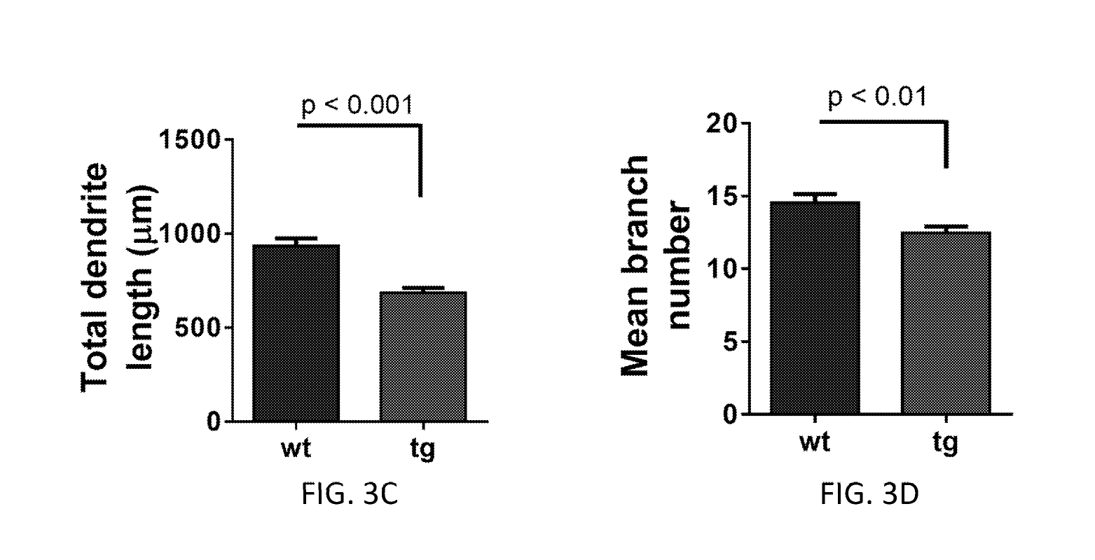

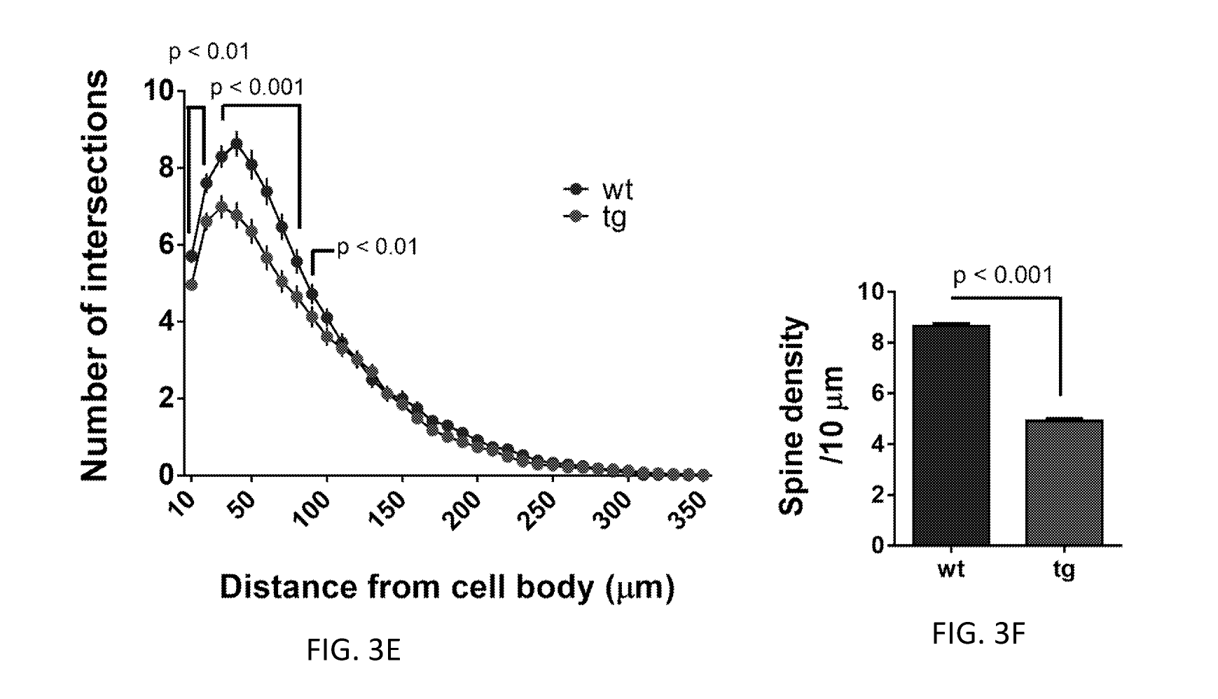

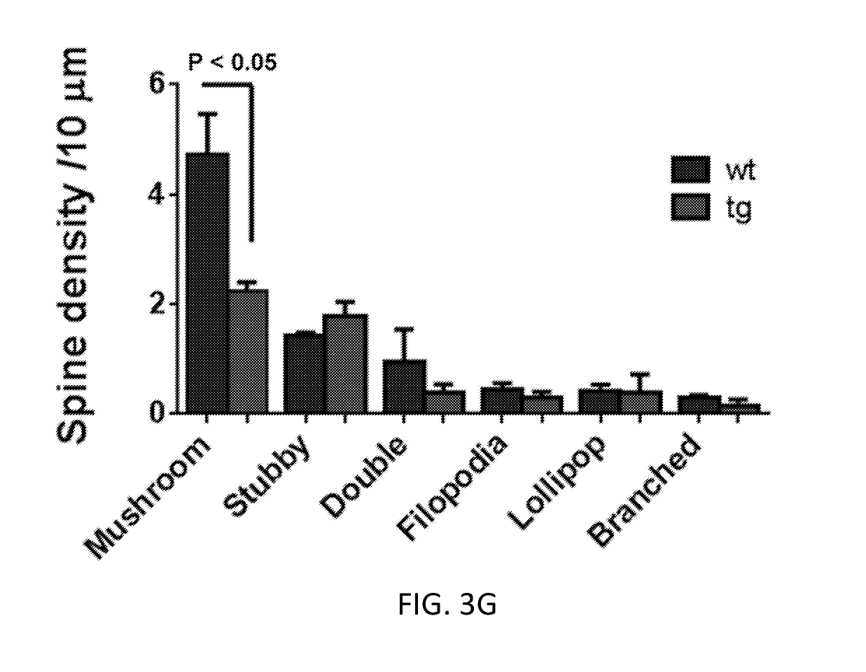

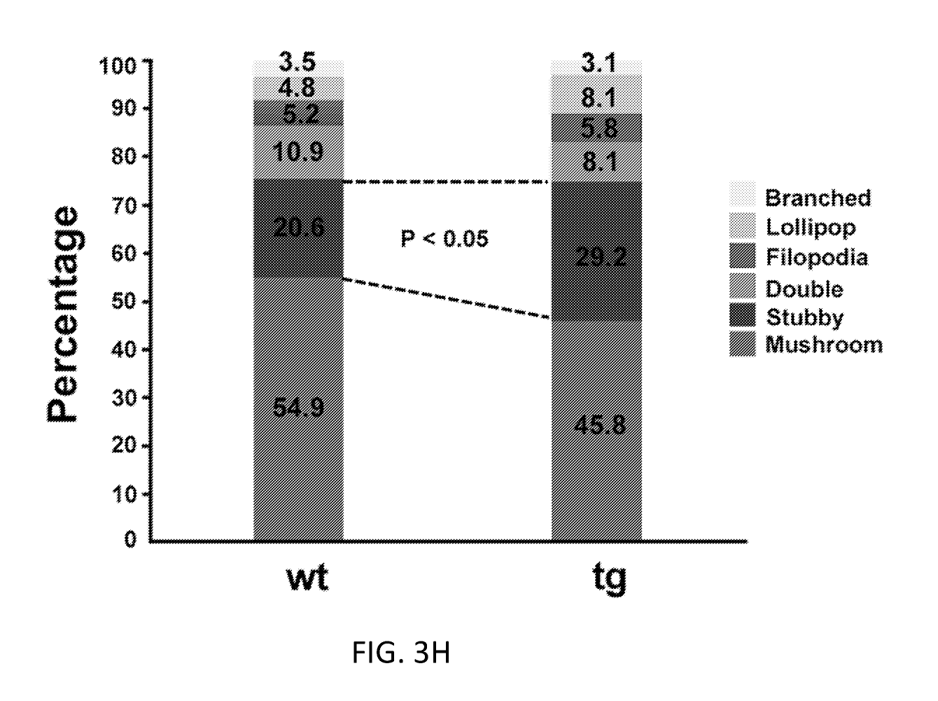

All experiments were performed on a single heterozygous line. HERV-K env transcripts were analyzed by gel electrophoresis and real-time PCR (FIGS. 3D and 3E). HERV-K env protein was confirmed by Western blot analysis (FIG. 3C) and following immunostaining for the HERV-K env which showed staining in cortical neurons. For all studies in this manuscript littermate animals without the transgene were used as controls. All experiments involving mice were performed according to the recommendations in the Guide for the Care and Use of Laboratory Animals of the National Institutes of Health (NIH). Mice were housed in a pathogen-free barrier facility with a 12-h light, 12-h dark cycle and ad libitum access to food and water. Both male and female animals at the age of 6 weeks, 3 to 9 months were used. The sample size for each experiment was determined on the basis of previous experiences with the transgenic animal models Quantification of data from all experiments involving mice was done by an investigator blinded to the genotype of the animals.

### Immunohistochemistry and Confocal Microscopy in Transgenic Animals.

Six to nine month old mice were deeply anesthetized with ketamine (100 mg/kg) and xylazine (10 mg/kg) i.p. and perfused transcardially with saline followed by 4% (w/v) paraformaldehyde (PFA). After postfixing in PFA overnight, brains and spinal cords were immersed in a 30% (v/v) sucrose solution. On the following day, brains and spinal cords were cryoprotected and cut in the coronal or horizontal plane into 40 μm thick sections on a sliding microtome. Sections were washed in Tris-Buffered Saline (TBS) (10 mM Tris-HCl, pH 7.5, 150 mM NaCl). Endogenous peroxidase activity was blocked with 3% (v/v) hydrogen peroxide, prior to incubation in blocking solution (TBS with 0.5% (v/v) Triton-X and 2.5% (v/v) donkey serum). Mouse anti-HERV-K env (1:500; Austral Biologicals, San Ramon, Calif.), mouse anti-NeuN (1:500; Millipore, Billerica, Mass.), rabbit anti-BCL11B (1:500; Novus Biologicals, Littleton, Colo.) or rabbit anti-Choline Acetyltransferase (anti-ChAT, 1:1000; Millipore) was applied overnight at 4° C. Biotinylated goat anti-mouse IgG or goat anti-rabbit IgG was used as the secondary antibody. Antibody binding was developed using a Vectastain® Elite ABC kit (Vector Laboratories, Burlingame, Calif.) and visualized with 3,3′-diaminobenzidine (DAB; Vector Laboratories). Sections were counterstained with hematoxylin (Dako, Carpinterina, Calif.). Images were processed using a whole slide scanner, Aperio (Leica Biosystems). For confocal microscopy, primary antibodies were diluted in blocking solution as follows: Mouse anti-HERV-K env (1:500; Austral Biologicals), mouse anti-NeuN (1:500; Millipore), mouse anti-Nucleophosmin (NPM, 1:500; Millipore), mouse anti-phospho-histone H2A.X (1:500, Millipore) and rabbit anti-GFAP (1:2000, Dako). Secondary antibodies were conjugates of Alexa Fluor 488 or Alexa Fluor 594 (1:250, Invitrogen, Grand Island, N.Y.) followed by washing and counterstaining with DAPI to label all nuclei. All images were obtained using a LSM 510 META laser-scanning confocal microscope (Carl Zeiss, Jena, Germany). For quantification, positively labeled cells were counted in at least three 200×200 μm fields from the selected brain regions for each animal evaluated to assess protein expression levels.

### Multi-Channel Fluorescence Microscopy.

Multi-channel wide field fluorescence microscopy was performed on 10 μm thick mouse brain coronal sections were immunoreacted for 1 hr at room temperature using a mixture of the following primary antibodies: mouse IgG2a anti-HERV-K env (1:200, Austral Biologicals), guinea pig IgG anti-NeuN (1:500, Millipore), mouse IgG1 anti-NeuN (1:500, Milipore), chicken IgG (IgY) anti-GFAP (1:500, Abcam, Cambridge, Mass., USA) and rabbit IgG anti-Iba1 (1:200, Wako Chemicals, Richmond, Va., USA). The sections were then washed in PBS and 0.5% BSA and immunoreacted using a mixture of the following fluorochrome-conjugated secondary antibodies: goat anti-mouse IgG2a-Alexa Fluor 647 (1:200, Invitrogen), goat anti-guinea pig IgG-Alexa Fluor 488 (1:200, Invitrogen), donkey anti-chicken IgG (IgY)-IRDye 800CW (1:100, Li-Cor Bioscience, NE, USA), and goat ani-rabbit IgG-Alexa Fluor 594 (1:200, Invitrogen). The sections were then washed in washing buffer, incubated for 5 min at room temperature in 1 mg/ml DAPI (Invitrogen) to stain cell nuclei, rinsed in distilled water, air dried and finally coverslipped using Immu-Mount medium (Thermo Fisher Scientific). All sections were imaged using a Axiovert 200M fluorescence microscope (Carl Zeiss) equipped with a 20× Plan-Apochromat (Phase-2) objective (Carl Zeiss), a high resolution ORCA-ER cooled digital camera (Hamamatsu Photonics, Japan) sensitive to a wide-spectrum of emission wavelengths, including those approaching infrared, a 100 W mercury arc lamp (Carl Zeiss), and excitation/dichroic/emission filter sets (Semrock, N.Y., USA) optimized to detect the following fluorophores: DAPI, Alexa Fluor 488, Alexa Fluor 594, Alexa Fluor 647 and IRDye 800CW. Each labeling reaction was captured using filtered light through an appropriate fluorescence filter set and the images individually digitized at 12-bit resolution using the Volocity imaging program (Improvision, Waltham, Mass., USA). An appropriate color table was applied to each image to either match its emission spectrum or to set a distinguishing color balance. The pseudocolored images were then converted into TIFF files exported to Adobe Photoshop and overlaid as individual layers to create multi-colored merged composites.

### Statistical Analyses.

No statistical methods were used for predetermining sample size. For animal studies, the sample size was based on prior experience with transgenic animals. Because animal groups were defined by genetic status, no randomization was used. The investigators were blinded to group allocation during the experiment and when assessing the outcome. Due to profound effects in the transgenic animals, blinding for analysis was not possible for motor tasks, or for histological analysis of spinal cord and muscle. For this reason, the histological data for spinal cord and muscle are provided as descriptive analyses. All other data were analyzed with GraphPad Prism v.6 (GraphPad, San Diego, Calif.). The Shapiro-Wilk test of normality was applied to all data sets to determine if a data set is normally distributed. The F test or Bartlett's test was for equality of variances between groups. Differences between means were assessed by paired two-sided Student's t test or one-way ANOVA followed by post-hoc testing. In cases where the data did not demonstrate a normal distribution, the Mann-Whitney test or Kruskal-Wallis test was used. Pearson's correlation coefficients were used to quantify the linear relationship between two variables. All available samples or animals were included for statistical analysis.

### RNA-Seq Analysis.

Libraries for six RNA samples from the brain with RNA integrity values of 9 or 10, representing three controls and three ALS subjects were prepared from 10 μl each following the Illumina mRNA-Seq protocol with ribosome depletion but without poly A+ enrichment. Prepared libraries were multiplexed and paired-end sequenced (101 bp) by NISC (http://www.nisc.nih.gov/) using one lane of an Illumina HiSeq 2000. Post CASAVA deplexing, fastq files were import into the CLCbio Genomics Workbench (www.clcbio.com) and subject to sequence adaptor removal, quality inspection, trimming, filtering, and mapping. Per trimming, 15 bp from the 5′ end of each read for each sample was globally removed along with 1 bp from the 3′ end. Bases with a call accuracy <95% were also removed. Per filtering, read pairs having more than two ambiguities in at least one read were discarded along with pairs <85 bp in length. Per mapping, the “Map to Reference” tool was used to align reads in pairs by sample against the human genome (GRCh37/hg19) without masking for five separate instances. With each instance, the “minimum percent reference similarity” criterion and “minimum percent read length” criterion were incremented by 10% together starting at 50%. Post mapping, the number of reads falling in “H/ERV” regions annotated by RepeatMasker (Library 20120124), Annotated “HERV-K” regions were enumerated and organized by sample by mapping instance. Counts were then converted into number of “Reads Per Kilo base of transcript per Million mapped reads” (“RPKM”). Where after, RPKM values were pedestalled by 2, log (base 2) transformed, filtered to remove regions not having a transformed value >1 for at least one sample, then quantile normalized. Normalized values for annotated regions were finally subset and the difference of means between ALS and control subjects calculated both by region and using the aggregate expression across all regions. Significance of these differences was tested using the Welch-modified t-test under corrected (Benjamini Hochberg) and uncorrected conditions.

### In Vitro Neurotoxicity Assay

Targeted and parent line neural stem cells from the NCRM1 line were ubiquitously expressing tdTomato and were cultured and maintained as previously described. Briefly, the cells were maintained in neural stem cell medium (NSCM) consisting of Neurobasal base medium supplemented with GlutaMAX, NEAA, 1×B27 (all from Life Technologies, Grand Island, N.Y., USA), and 10 ng/mL beta-fibroblast growth factor (Peprotech, Rocky Hill, N.J., USA). Media was changed every other day and cells were passaged using accutase about every 4 days. Neuronal differentiation was accomplished as previously described (36) with some adjustments. Briefly, neural stem cells were passaged to about ˜70% confluence in 6-well plates and cultured in neuronal differentiation medium (NDM) for ten days. NDM consisted of DMEM/F12, GlutaMAX, 1% BSA, 1× human embryonic stem cell supplement, brain derived neurotropic factor at 10 ng/mL, and glia derived neurotropic factor at 10 ng/mL (Peprotech). NDM was changed every two days. On day 7 of neuronal differentiation, cells were passaged using Accutase and electroporated with no plasmid added (sham transfection), or transfected with 10 μg pcDNA control plasmid or HERV-K env plasmid according to the manufacturer's instructions (Lonza, Walkersville, Md.; Nucleofector 2S). pcDNA was used as a control because the HERV-K was introduced in this backbone. Transfected cells were then plated on Geltrex-coated 96-well plates at a ratio of ˜3 million cells per plate. The neurons were imaged every 24 hours on the GE INCell Analyzer 2000 high content imager at both 10× and 40× magnifications, acquiring images at 4 fields per well and 18 wells per treatment group. NDM was then changed every two days by removing 30% of the medium and replacing it with 50% of fresh NDM. The effects of mock transfection, pcDNA control and HERVK env transfection of the neurons were quantitated using GE Investigator Developer Toolbox software. Neuritic length of the cultures and cell count was determined in 18 wells of each treatment group. Another set of human neuronal cultures were treated with 3 mM 3-nitropropionic acid, 100 μM N-methyl-D-aspartate or 100 μM hydrogen peroxide and monitored for cell numbers and neurite length for 18 hours as described above. From a set of cultures run in parallel, RNA was extracted and analyzed for HERV-K gag and env transcripts as described above. Values represent mean±SEM (3 experiments per condition) and were analyzed by one-way ANOVA followed by Newman Keuls post hoc comparisons.

### Magnetic Resonance Imaging

9 months old wt (n=5) and tg (n=5) mice were subjected to MR imaging. Mice were perfused and fixed with 4% paraformaldehyde containing 0.5% Magnevist. The brains were then extracted and incubated in a same solution overnight for a post fixation. MR imaging was carried out in the NIH Mouse Image Facility. MR images were acquired on a 14 Tesla Bruker Biospec NMR spectrometer with microimaging gradients (Bruker Biospec, Inc., Billerica Mass., USA) using a 3D FLASH sequence with the following parameter values; TE 3.9 ms, TR 50 ms, average number 1, Field-of-View of 22×14×11 mm and an acquisition matrix 512×325×256 (37). The image resolution is 43 microns isotropic over the entire brain. The MRI scans were stored into a Digital Images and Communications in Medicine (DICOM) image files and transferred to Medical Image Processing, Analysis and Visualization (MIPAV) software, where manual volumetric analysis was carried out. MIPAV allows the user to manually outline regions of interest (ROIs) and afterwards calculates the volumes of a specific ROI.

This study focused on measures of six brain regions: whole brain volume, motor cortex thickness and volume, cingulate cortex volume, corpus callosum thickness and hippocampus volume. ROIs were traced without reference to the genotype of the mice. Brain volumes were calculated by multiplying the thickness of the slice (0.04 mm) by the area of the cross-section. The total volume for the region was calculated by summing these slice volumes. The perimeter of the entire rostral-caudal length of the brain was traced to calculate whole brain volume. This included everything from the slice most similar to that labeled as Bregma+4.28 mm in a mouse brain atlas to the slice most similar to that labeled Bregma −8.24 mm. The starting point was the beginning of the olfactory bulb, and the endpoint was the transition from the brainstem to the spinal cord. The motor cortex of mouse brain starts to appear at around and to its most dorsolateral point. This point was then connected with a line to the most dorsal and medial intra-hemispheric point of the cortex. Using this landmark-based method, the cingulate cortex was measured on nine slices starting rostrally at the closure of the genu of the corpus callosum (approximately Bregma+1.09 mm from Bregma) and terminating caudally at the rostral limit of the hippocampus (approximately −0.51 mm from Bregma). A line between its most dorsal point and its most ventral point at the intersection of the corpus callosum with the midline was drawn and measured to determine the corpus callosum thickness. Nine cross-sections of MRI scans were measured between those labeled as Bregma +1.09 mm and Bregma −0.51 mm. The hippocampus (CA) and dentate gyrus (DG) were manually outlined on cross-sections at the levels, approximately Bregma −1.55 mm, −1.91 mm and −2.27 mm.

### Behavioral Analysis

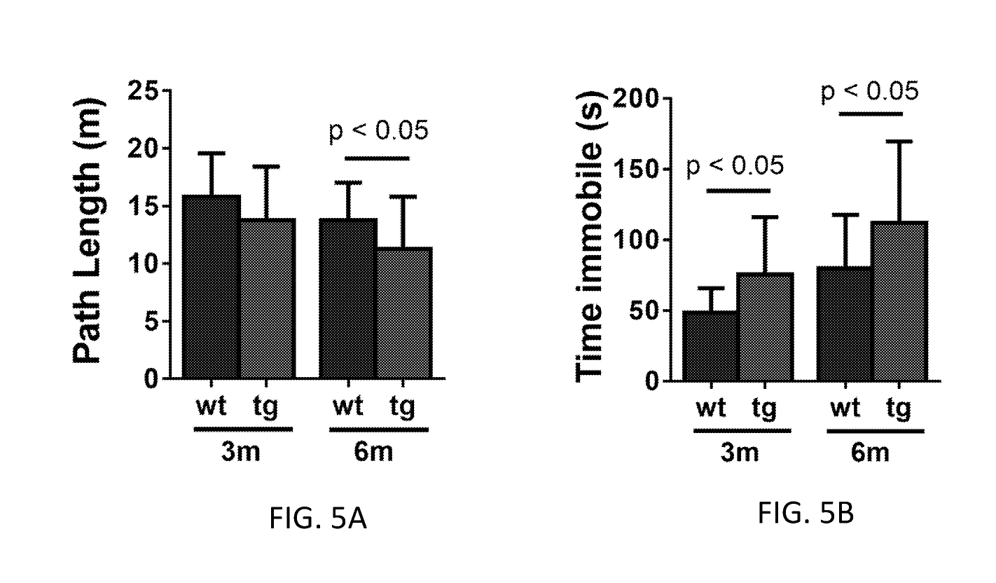

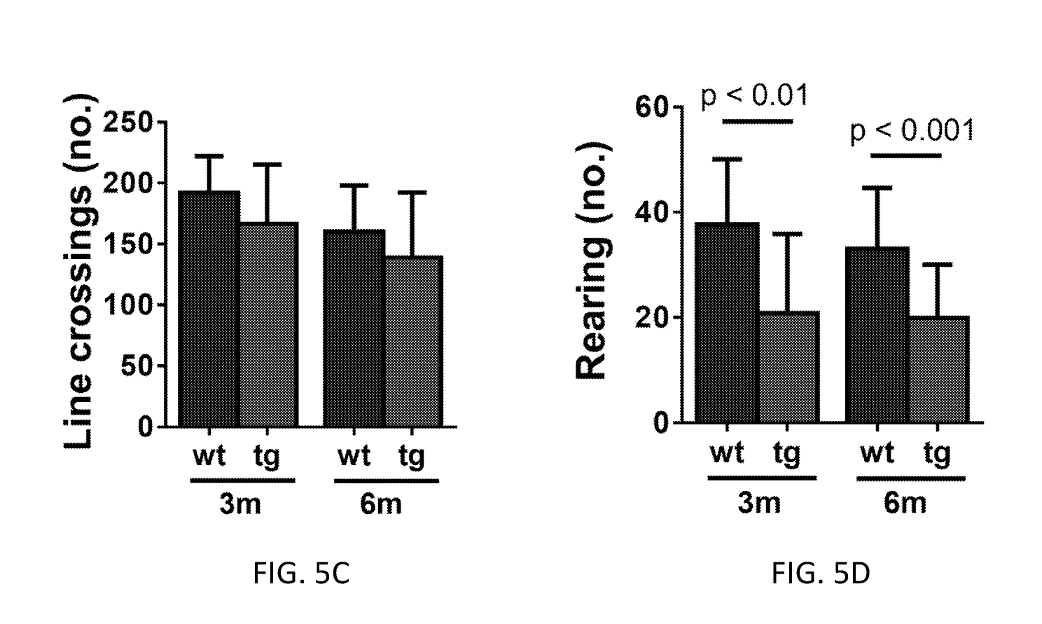

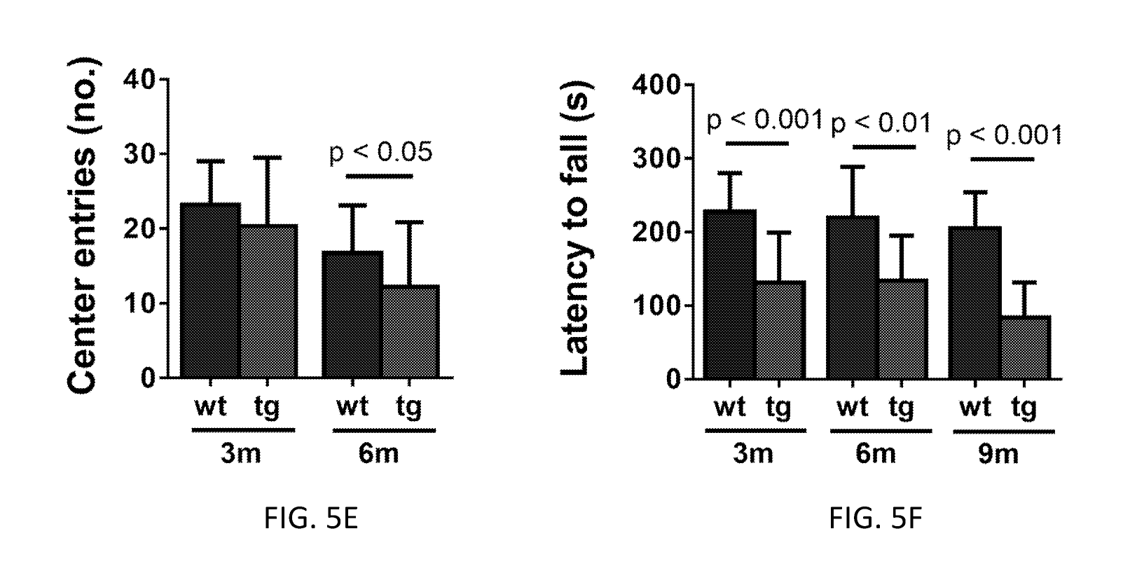

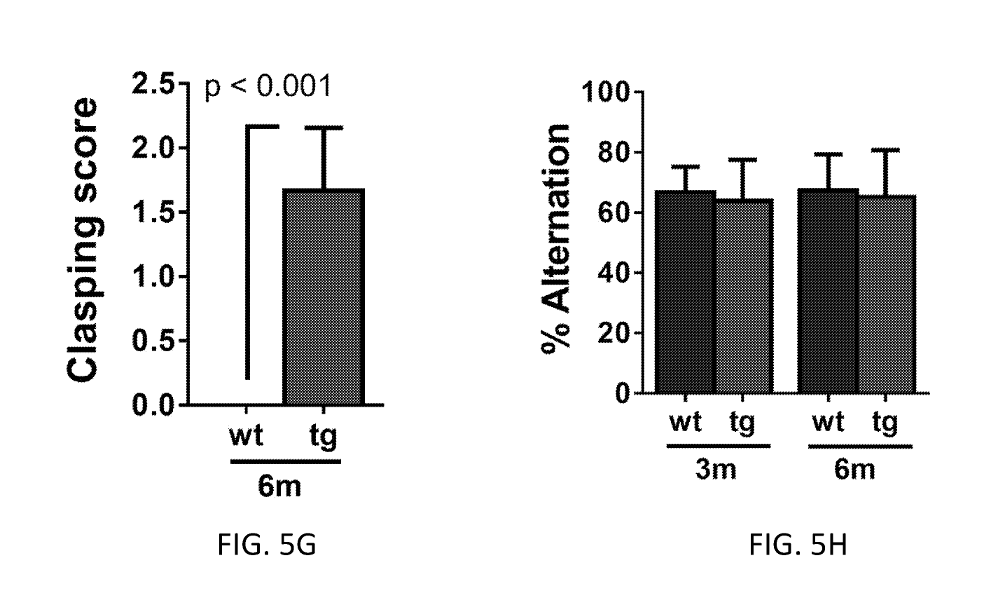

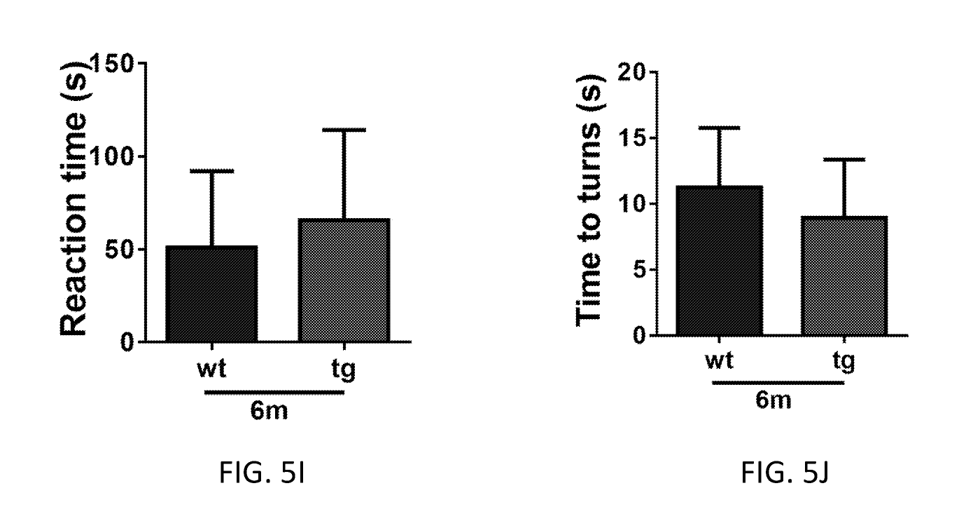

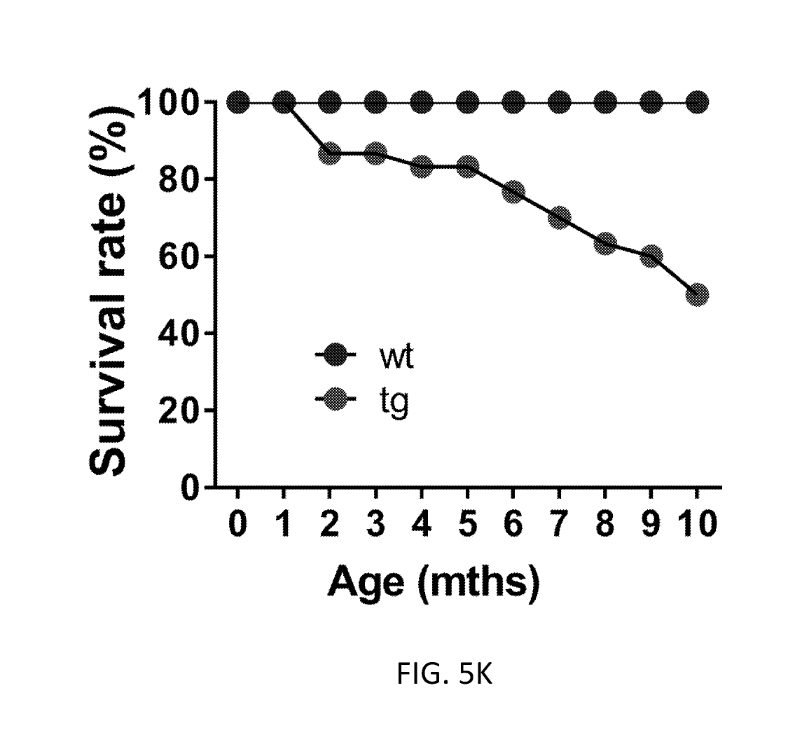

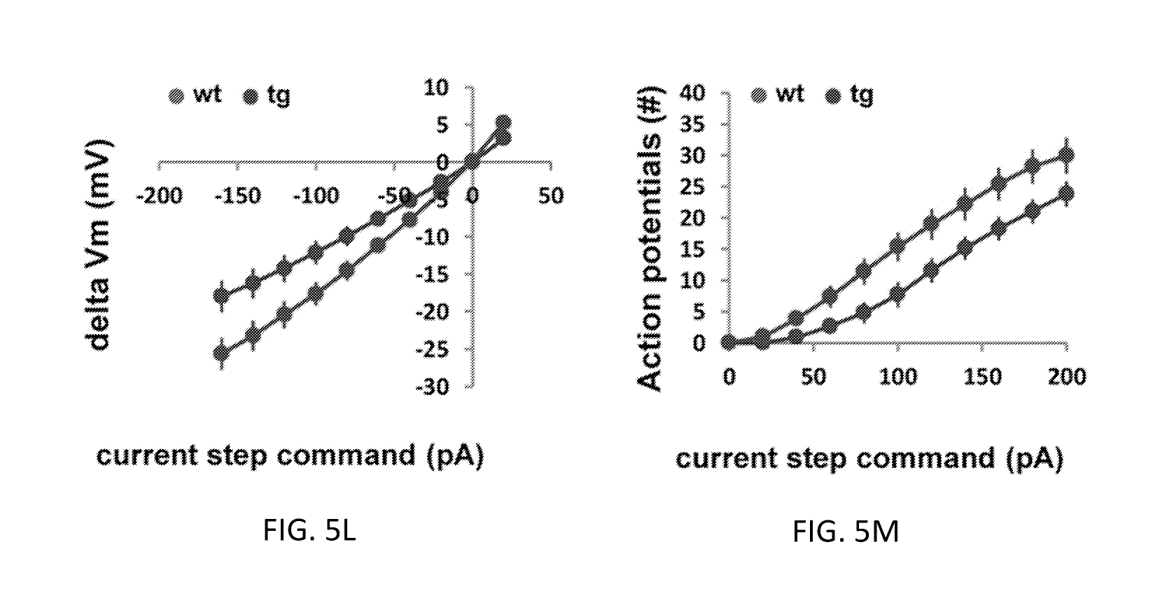

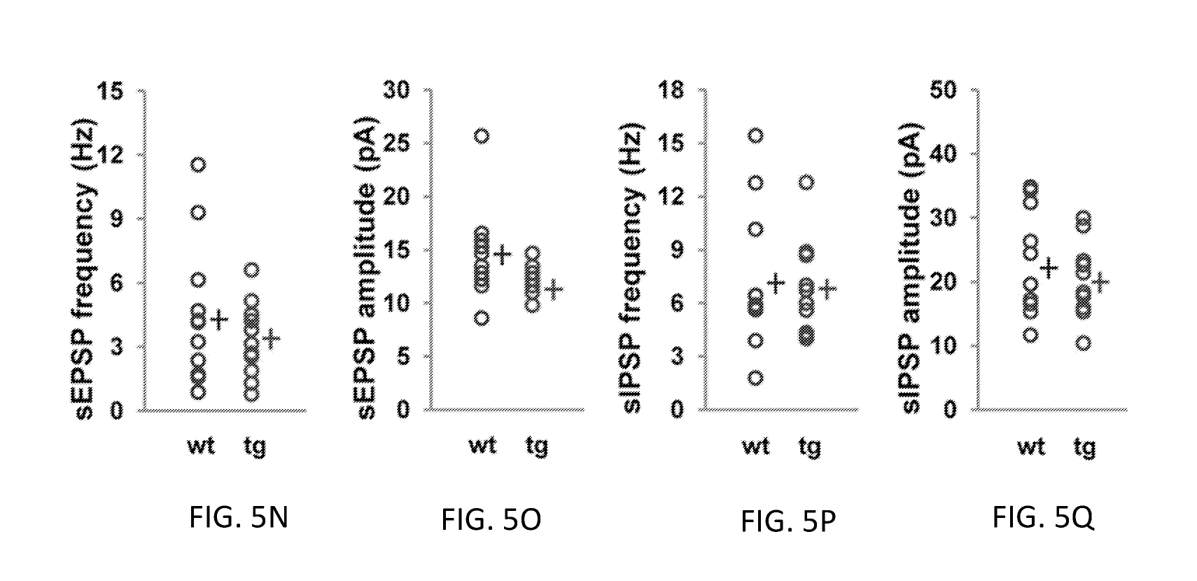

Both male and female mice at 3 to 9 months of age were used. Mouse behavior was tracked using a video tracking software ANYmaze™ (Stoelting Co., Wood Dale, Ill.). Animals were returned to their cage during the inter-trial intervals and after the completion of each paradigm. All behavioral tests were conducted between 10 a.m. and 3 p.m. by an investigator blinded to the condition of the animal. However due to the prominent phenotypic changes in the tg animals such blinding was not successful in the older animals. Values of all behavioral analyses are presented as mean±SEM. Sample sizes for animal used for each test are provided in the legend for FIG. 5. Data was analyzed by Student's t test. All behavioral tests were conducted in a blinded manner. However due to the prominent phenotypic changes in the tg animals, such blinding was not successful in the older animals for motor tasks.

Open Field Task:

The open-field test was used to evaluate general locomotor activity, novel environment exploration and anxiety-like behavior in wt and tg mice. The open field was carried out in a square chamber of 40 cm2 surface area and 35 cm high walls. It was divided equally into 16 squares by infrared photocell beams. The central 4 grids were considered to be the center, and the rest were assigned as the periphery. An automated system recorded each beam break as one unit of exploratory activity, similar to manual scoring of each line crossed. Mice were placed into a corner of the arena and were then allowed to freely explore the arena for 5 min. Their movement broke laser beams, and the tracking system automatically recorded each beam break as one unit of activity. During the observed time period, the number of grid lines crossed, vertical movements, latency to enter the center, time spent in the center and number of times the animal entered the center were recorded.

Tail Suspension Test:

Motor dysfunction was also assessed by monitoring clasping of the limbs, triggered by a tail suspension test. The clasping score was assessed as previously described. Mice at 6 months of age were suspended by the tails for 15 sec and the movements of hind limbs were observed. Mice were assessed by a clasping score. The score was rated 0 if no clasping was observed during a period of 15 sec, 1 if abnormal extension of the hind limbs was noticed, 2 if mouse started to clasp, and 3 if clasping it was firmly established.

Y-Maze Task:

The Y-maze test assessed working memory by monitoring spontaneous alternation behavior in a Y-shaped maze. The apparatus was made of 3 acrylic plastic arms (35 cm length, 5 cm width, and 10 cm height) at 120 degrees to each other. Mice were placed in the center of the maze and were allowed to freely explore the three arms for 8 min. The maze activity was recorded via camera-based tracking system (Stoelting). To measure spontaneous alternation, the number of arm entries and the number of alternations were scored. Arm entry was defined as entry of all four paws of the mouse within the arm. Consecutive entries into three different arms were defined as alternations. Alternation percentage was calculated by dividing the number of alternations by the number of possible alternations and then multiplying by 100.

Sticky Paper Test:

This somatosensory test was performed as previously described. The animals were acclimated to the testing box (30×45 cm) for 1 min. Self-adhesive tape strips (0.3×0.4 cm) were placed onto the ventral side of the hind paw of mice. Animals were then replaced in the testing box and the performance on the task was video recorded and analyzed off-line. The latency of the first reaction to the stimulus (paw lifting, sniffing, biting, or removal) was measured.

Negative Geotaxis Test:

Mice were placed on the inclined platform of 45 degree facing in a downward direction. The latency to turn and orient head-up from downhill initial position was video-recorded. Delays in the ability to reorient are indicative of delays in vestibular dysfunction.

### In Utero Electroporation

Timed-pregnant female CD-1 mice from Charles River at embryonic day 14 were anesthetized with ketamine/xylazine (100/10 mixture; 0.1 mg/g body weight, i.p.). The uterine horns were exposed. A lateral ventricle of each embryo was injected with a mixture of plasmid DNA encoding HERV-K env (2 μg/μl) and tdTomato expression vector (1 μg/μl) or tdTomato expression vector alone (1 μg/μl) with a glass micropipette made from a microcapillary tube (Sutter Instrument Co., Novato, Calif., USA). Injected plasmid solution contained Fast Green solution (0.001%) to monitor the injection. The embryo's head in the uterus was held between the tweezers-type electrode, consisting of two disc electrodes of 5 mm diameter (CUY650-5, Nepa Gene Co., Chiba, Japan). The positive electrode was placed on the dorsal lateral side of the brain to target the cerebral cortex. According to the manufacturer's protocol, electrode pulses (35V, 50 ms) were charged 4 times at intervals of 950 ms with an electroporator (CUY21SC, Nepa Gene Co., Ichikawa-City, Japan). The uterine horn was placed back into the abdominal cavity and the abdominal wall and skin was sutured. At postnatal day 14 the brains were removed, fixed and sectioned for imaging in mice electroporated with env DNA or a control DNA constructs. All images were obtained using a LSM 510 META laser-scanning confocal microscope (Carl Zeiss).

### Golgi Staining:

SuperGolgi Kit (Bioenno, Santa Ana, Calif., USA) was used to perform Golgi staining following the vendor's protocol. Briefly, the freshly dissected brains from wt (n=3) and tg (n=3) animals at the age of 6 months were immersed in impregnation solution and stored at room temperature for 10 days in the dark. The brains were then transferred into post-impregnation buffer and kept for 48 hours in the dark. They were sliced using a vibratome (VT1200 S; Leica, Nussloch, Germany) at a thickness of 100 μm and stained using standard procedures. For dendritic morphological analysis, cortical neurons, primarily in layer V were analyzed by observing the dendrites, ensuring that they showed a completely impregnated dendritic tree and uncut dendrites and were relatively isolated from neighboring cells. Neurons that had truncated and/or non-tapering dendrites were not included in the analysis. For each selected neuron, all branches of the dendritic tree were traced at 20× magnification. Total length of dendrite trees and number of dendritic branches were measured using NIH Image J with Neuro J plugin. The complexity of dendritic trees was also assessed by Sholl analysis. Accordingly, the number of intersections of dendrites was calculated with concentric spheres positioned at radial intervals of 10 μm as previously described. Fifty cortical neurons from each mouse were analyzed.

For quantification of spine density, 10 μm segments of secondary dendrites that ended in a fine taper were captured from cortical neurons. To maintain consistency in analysis of spine areas we chose the segments of dendrites located 10-30 μm from the tip of each dendritic branch. 30 dendrites from each mouse were identified. To further assess the HERV-K env-induced changes in spine morphology, the protrusion of dendritic spines was categorized into 6 types based on their shapes and length; mushroom spines have small necks and a large head and are usually seen in mature synapses; stubby spines have no necks, are short and wide in shape and considered to represent either a transitional growth stage between an early to mature spine or a stage during retraction of the mature spine for elimination; double spines have a neck protruding from the parent dendrite; filopodia spines have long thin protrusions with no obvious head and represent an early stage of spine formation; lollipop spines have thin necks and small heads and branched spines have two oblique necks ending as bulbous heads emerging from a common single neck. We compared the distribution of dendritic protrusions between groups.

Disconnected dendritic beads and swellings are hallmarks of dendritic injury. To observe the dendritic morphologies of Golgi impregnated cells, high magnification (63×) images were captured in the cortical neurons of wt and tg mice.

### Electrophysiology

Six weeks old wt and tg mice were decapitated under isoflurane anesthesia and the brain was immediately excised and immersed in ice-cold solution containing (in mM): 90 sucrose, 80 NaCl, 1.3 KCl, 1 NaH2PO4, 25 NaHCO₃, 2 CaCl2), 1 MgCl2, 10 mM glucose, 3 mM pyruvic acid; pH 7.2-7.3; 310 mOsm/l). Coronal slices (300 μm) containing the mPFC were cut with a vibrotome (Leica VT1200S). After recovery, incubation for ˜15 min at 33° C. was followed by ˜45 min at 22° C. in artificial cerebrospinal fluid (ACSF; in mM: 125 NaCl, 2.5 KCl, 1 NaH2PO4, 25 NaHCO3, 2 CaCl₂), 1 MgCl2, 20 glucose, 3 mM Pyruvic acid; pH 7.2-7.3; 310 mOsm/l). Slices were then transferred to the recording chamber and superfused (2-3 ml min-1) with ACSF at 32-33° C. All solutions were saturated with 95% 02, 5% CO2. Whole-cell patch recordings were obtained from pyramidal neurons in layer 5 of the medial prefrontal cortex. The animals were coded so that electrophysiological experiments and analyses were performed blind with respect to genotype.

Current Clamp:

To measure intrinsic properties (input resistance and excitability) currents steps (1 s, −160 to +220 pA/20 pA step) were injected and all cells which were maintained at −65 mV after membrane break-in and measurement of the resting membrane potential. Recordings were performed in the presence of 20 μM NBQX and 50 μM picrotoxin in the bath to block glutamatergic and GABAergic transmission, respectively and recording pipettes were filled with an internal solution containing (in mM): 125 K-gluconate, 20 KCl, 10 Hepes, 4 NaCl, 0.5 EGTA, 4 Mg ATP, 0.3 GTP, 10 phosphocreatine (pH 7.2, 290 mOsm).

Voltage Clamp:

Spontaneous excitatory synaptic activity was recorded near the reversal potential of inhibition (−60 mV) or of excitation (0 mV) and patch electrodes were filled with (in mM): 125 K-gluconate, 4 KCl, 4 NaCl, 10 Hepes, 0.5 EGTA, 4 Mg ATP, 0.3 GTP, 10 phosphocreatine (pH 7.2, 290 mOsm).

Drugs:

All chemicals were purchased from Tocris (Ballwin, Mo.), Abcam (Cambridge, Mass.) or Sigma Chemical (St. Louis, Mo.).

Data Acquisition and Analysis:

Electrophysiological recordings were obtained using a multiclamp 700B amplifier and PClamp 10 (Molecular Devices, Sunnyvale, Calif.). Data were analyzed using Microsoft Excel, Minianalysis (Synaptosoft, Decatur, GA), and/or IGOR Pro (WaveMetrics, Lake Oswego, Oreg.). Pooled data are presented as either mean±SEM or box plots.

### Product Enhanced Reverse Transcriptase (PERT) Assay

PERT assay was used as described with minor modifications. Briefly, cell culture supernatant was collected and centrifuged to pellet any cell debris. The cleared supernatant was then supplemented with 0.25% Triton X-100, 5 mM dithiothreitol and 0.25 mM ethylene-diamine-tetra-acetate as the source of HERV-K RT. Bacteriophage MS2 genomic RNA was used as template for the reverse transcription reaction. Quantitative PCR was performed with TaqMan primers (MS2-Forward and MS2-Reverse) and probe (MS2-Probe) using Applied Biosystems Vii 7. Reverse transcriptase activity was expressed as fold change compared to control or as pg/ml RT determined by standard curve generated from PERT using HIV-1 RT. Primers used are shown in the following table:

| Target gene | Primer sequence (5′ to 3′) | |
|---|---|---|
| HERV-K env | Forward: | CTGAGGCAATTGCAGGAGTT (SEQ ID NO: 17) |
| | Reverse: | GCTGTCTCTTCGGAGCTGTT (SEQ ID NO: 18) |
| HERV-K gag | Forward: | AGCAGGTCAGGTGCCTGTAACATT (SEQ ID NO: 21) |
| | Reverse: | TGGTGCCGTAGGATTAAGTCTCCT (SEQ ID NO: 22) |
| GAPDH | Forward: | TGCACCACCAACTGCTTAGC (SEQ ID NO: 29) |
| | Reverse: | GGCATGGACTGTGGTCATGAG (SEQ ID NO: 30) |
| MS2 | Forward: | TCCTGCTCAACTTCCTGTCGA (SEQ ID NO: 35) |
| | Reverse: | CACAGGTCAAACCTCCTAGGAATG (SEQ ID NO: 36) |
| | | [6FAM]CGAGACGCTACCATGGCTATCGCTGTAG[TAM] (SEQ ID NO: 37) |

### Luciferase Assay

All cell lines were obtained from ATCC and were tested for mycoplasma contamination prior to use. HeLa cells were seeded into 24-well plates 24 hours before transfection. Cells were transfected with pMetLuc-HEVRV-K-LTR and pcDNA3.1-TDP43 or other plasmids as indicated. 48 hr later, supernatants were collected and assayed for luciferase activity according to manufacturer's instruction (Clontech, Mountain View, Calif.). Relative luciferase activity was expressed as % relative luciferase units (RLU) for fold change relative to control.

### Transfection of Cells

Transfections were performed using Lipofectamine 2000 reagent according manufacturer's instructions (Life Technologies, Grand Island, N.Y.). For knockdown experiments, 50 nM ON-TARGETplus SMARTpool siRNA specific for TDP-43 (Thermo Scientific, Rockville Md.) was transfected into cells using Lipofectamine siRNAmax (Life Technologies). Cells were harvested at 48 hr post-transfection, total RNA was extracted and HERV-K gag or pol transcripts were analyzed by RT-PCR. Neuronal cells were nucleofected with pcDNA control plasmid, HERV-K whole genome expression plasmid or TDP-43 construct using program DN-100 on the 4D-Nucleofector system (Lonza). Transcripts were expressed relative to β-actin or GAPDH endogenous control, as indicated in figure legends. Data represent at least three independent experiments. Values are shown as mean±SEM and analyzed by Student's t test.

### Biotin-Streptavidin DNA-Protein Immunoprecipitation Assay

Nuclear extracts (NE) from 293T cells were isolated using the NE-PER nuclear and cytoplasmic extraction kit, according to the manufacturer's protocol (Thermo Scientific). NE (100 μg) were incubated with 300 ng of biotinylated DNA probe (corresponding to putative TDP-43 binding sites) for 15 min at room temperature. Streptavidin-conjugated magnetic Dynabeads (Invitrogen) were added and the mixture was incubated for an additional 30 min with slow mixing. Beads were pre-incubated with 1.0% bovine serum albumin (BSA) in phosphate-buffered saline (PBS) for 45 min prior to their addition to block non-specific interactions. The beads were then washed three times under low-stringency (PBS, 137 mM NaCl) or high-stringency conditions (10 mM Tris, pH 7.5, 1 mM EDTA, 300 mM NaCl) and resuspended in SDS loading buffer for western blot analysis. Equal volumes were loaded and run on a 4-12% (w/v) Bis-Tris electrophoresis gel (Life Technologies). The proteins were then transferred onto a PVDF membrane and immunoblotted with an antibody against TDP-43 (Abcam). Optical density of the bands was measured and expressed relative to that obtained from TDP-43 binding to nt726. Values represent mean+SEM of three independent experiments.

### Western Blot Analysis, Immunofluorescence and Antibody Production

For Western blot analysis of HERV-K viral protein expression and cleavage, 293T cells were transiently transfected with either the HERV-K expression vectors or empty vector using lipofectamine 2000 (Invitrogen). After 48 hrs transfection, cells were washed with PBS and lysed in radioimmunoprecipitation assay (RIPA) buffer containing protease inhibitors (Roche). The insoluble pellet was removed by a 10 min centrifugation at 12,000×g. The harvested lysates were separated by sodium dodecyl sulfate polyacrylamide gel electrophoresis using Novex 4 to 12% Bis-Tris gels (Invitrogen), followed by transfer onto polyvinylidene fluoride membranes. The blots were incubated overnight at 4° C. with either anti-HERV K Env antibody (Austral biologicals) or anti HERV K Gag antibody (Austral biologicals) followed by one-hour incubation with a secondary antibody linked to horseradish peroxidase. After 30 min washing, the blot was developed with SuperSignal™ West Femto ECL reagent (ThermoFisher), and imaged with FluoroM imaging machine (ProteinSimple). Immunofluorescence analysis for the co-localization of Gag and Pol was performed on 293T cells transiently transfected with the HERV-K expression vector or empty vector (negative control). 24 hrs post-transfection cells were fixed with 4% paraformaldehyde, permeabilized, and stained with a rabbit polyclonal anti-Pol serum and a mouse monoclonal anti-Gag antibody. Alexa 488-conjugated goat anti-rabbit IgG and Alexa 594-conjugated goat anti-mouse IgG were used as secondary antibodies (Molecular Probes); nuclei were stained with 4′,6-diamidino-2-phenylindole (DAPI; Molecular Probes).

A polyclonal anti-HERV-K Pol antibody using amino acids 57-245 as an immunogen was developed by SDIX with its proprietary Advanced GAT technologies. The monoclonal antibodies against the full length HERV-K Env and Gag were obtained from Austral Biologicals. Rabbit antisera against HERV-K envelope protein were developed by Genscript, using peptides QRKAPPRRRRHRNRC, CSDLTESLDKHKHKK, and CSKRKGGNVGKSKRD as immunogens.

### Chromatin Immunoprecipitation (ChIP) and Quantitative Real-Time PCR

HeLa cells at approximately 50% confluence were transiently transfected with HERV-K 5′LTR-luciferase construct or pcDNA3.1(+) control vector with or without plasmid encoding wild-type TDP-43. 48 hr post-transfection, cells were fixed in fresh 1.0% (w/v) paraformaldehyde and ChIP was performed using the Epigentek Chromaflash One-Step ChIP Kit with antibodies against RNA Polymerase II (phosph-S2; Abcam) or TDP-43 (Abcam). An isotype IgG control antibody (Epigentek, Farmingdale, N.Y.) was included in all experiments. Primers used to amplify HERV-K LTR were:

forward 5′-GTTTGTCTGCTGACCCTCTC-3′ (SEQ ID NO:40) and

reverse 5′-CCTGTGGGTGTTTCTCGTAAG-3′ (SEQ ID NO:41) to amplify a 231 bp region encompassing the transcription initiation site;

forward 5′-GGAAAGCCAGGTATTGTCCA-3′ (SEQ ID NO:42) and

reverse 5′-CTCCTCAGCACAGACCCTTT-3 (SEQ ID NO:43)′ to amplify a 120 bp region that encompassed nt343;

forward 5′-GGGCAGCAATACTGCTTTGT-3′ (SEQ ID NO:44) and

reverse 5′-TTCTCAAAGAGGGGGATGTG-3′ (SEQ ID NO:45) to amplify a 174 bp region that encompassed nt726 and nt761; and

forward 5′-CACATCCCCCTCTTTGAGAA-3′ (SEQ ID NO:46) and

reverse 5′-CTCGTAAGGTGGGACGAGAG-3′ (SEQ ID NO:47) to amplify a 174 bp region that encompassed nt866 and nt893.

Negative control reactions were also performed using primers for an unrelated sequence (GAPDH promoter):

| (SEQ ID NO: 48) | |
|---|---|
| forward | 5′- TACTAGCGGTTTTACGGGCG-3′ |
| and | |
| (SEQ ID NO: 49) | |
| reverse | 5′-TCGAACAGGAGGAGCAGAGAGCGA-3′. |

Real-time PCR was performed using the Fast SYBR Green Supermix kit (Life Technologies) in a ViiA 7 Real-Time PCR system (Applied Biosystems). Fold-change in binding was calculated by relative quantitation using the comparative threshold cycle (Ct) method, with results reported relative to control IgG (ΔCT=CTTarget−CTIgG control; fold change relative to IgG control=2−ΔCt). Values represent mean±SEM of three independent experiments.

### Isolation of Skeletal Mouse Muscle and Immunohistochemistry

Animals at the age of 6 months (n=5, wt and n=6, tg) were euthanized by CO2 inhalation after being anesthetized with ketamine (100 mg/kg) and xylazine (10 mg/kg) i.p. Skin of the right hind limb of mouse was pinched and peeled off to completely show leg muscles. The tibialis anterior and quadriceps were isolated along the bones and connective tissues, blood vessels, nerve bundles an adipogenic tissue were removed from the dissected muscle tissues. The muscle tissue was mounted using tragacanth gum, flash-frozen in dry-ice cooled 2-methylbutane, and stored at −80 C until cutting 10 μm cross-sections using a Leica CM1860 cryostat. Cryosections were blocked in PBS containing 5% bovine serum albumin (BSA) for 1 hr and labeled for 1 hr at room temperature with the following antibodies: rabbit anti-MYH7 (anti-myosin heavy chain I, 1:150; Sigma, St. Louis, Mo.) and mouse anti-BF-F3 (anti-myosin heavy chain Ilb, 1:20; Iowa City, Iowa). The sections were then washed in PBS and 1% BSA. Secondary antibodies were conjugates of Alexa Fluor 488 or Alexa Fluor 594 (1:200, Invitrogen) followed by washing and counterstaining with DAPI to label all nuclei.

### Recombinant Virus Production and Infection

293T cells were cultured in DMEM with 10% FBS and penicillin-streptomycin. Cells were transiently transfected in 10 cm plates at 5×106 cells/plate using lipofectamine 2000 (Invitrogen) according to the manufacturer's protocol. Briefly, cells were co-transfected with pCD-HK/Rev with or without pcDNA3.1-VSVG. After 24 hrs, the transfection media was completely removed and cells were washed with phosphate-buffered saline (PBS) to eliminate any residual plasmid and then fresh medium was added to the cells. Virus particle-containing supernatants were harvested after an additional 24 to 48 hrs and cleared of any cellular debris with two centrifugations at 1,000×g at 4° C. The clarified samples were then subjected to DNase treatment using the RNase-free DNase kit (Qiagen). Cleared supernatant was concentrated using Retro-X™ Concentrator (Clontech) as per manufacturer's instructions. Briefly, viral supernatant was mixed with the Retro-X Concentrator and incubated overnight at 4° C. The mixture was then centrifuged at 1500×g for 45 min at 4° C. to obtain a virus-containing pellet. The viral pellet was gently resuspended using complete DMEM and titrated using the PERT assay. An absolute amount of RT was determined using HIV RT as standard and 80 μg of HERV-K virus was used for each infection. At the time of transduction of target cells, the concentrated virus was again treated with RNase free DNase to ensure there was no plasmid DNA contamination. Infection was performed by exposing the resuspended DNase treated viral samples with fresh 293T or HeLa cells that had been plated in 24-well plates 24 hrs earlier in 5 g/ml of polybrene in the presence or absence of Abacavir, Zidovudine or Raltegravir. Total RNA was extracted six days post infection and HERV-K Gag gene expression was quantified using QPCR. Any change in RT inhibitor treated wells compared to untreated was expressed as percent inhibition.

### Toxicity Assay

HeLa cells were cultured in microplates (tissue culture grade, 96 wells, flat bottom) in a final volume of 100 μl/well culture medium in a humidified atmosphere (e.g., 37° C., 5% CO2). 24 hrs later, the cells were treated with HIV inhibitors at dosage ranging from 0.01 μM to 10 μM. Six days post-treatment Cell Proliferation Reagent WST-1 (Roche) was used per manufacturer's instructions to determine drug toxicity. Briefly, 10 μl of Cell Proliferation Reagent WST-1 was added to each well and the plate was shaken thoroughly for 1 min on a shaker. The cells were incubated for 0.5 to 4 hrs in a humidified atmosphere (37° C., 5% CO2). The absorbance of the samples against a background control as blank was measured using a microplate reader at 420-480 nm using a FlexStation microplate reader (Molecular Devices).

### Comparative Modeling

All the comparative modeling was performed using the homology modeling protocols implemented in the program Molecular Operating Environment (MOE). The sequences of target and templates were initially aligned with clustalW and manually adjusted after inspection to place insertions and deletions in favorable regions. An AMBER10HT force field was used for energy calculations and minimization. Ramachandran′ plot showed 95% of the residues of the final model are in allowed regions, and no rotamer outliers are present. When complexes between template and target inhibitors are available, the poses of the inhibitors in HERV-K targets were based on that of the template's complexes. When required to improve the pose, rotamers of selected residues 4.5 Å apart from the drug were explored to relieve the few clashes observed and to improve contacts, as well as drug and nearby residues relaxed by minimization. The drug minimization in the active site environment of the HERV-K targets was performed, tethering the protein atoms to their initial position with a weak harmonic potential (0.5 kCal/mol) during minimization.

In the case of the protease, the structures of a dimer of HIV-1 (PDBId: 2HS1, 0.85 Å) and Rous sarcoma virus (RSV; 1 BAI, 2.4 Å) proteases were used as templates. The RSV protease structure was used to model the insertion in the loop between β4-5 because it displays similar characteristics to the one in HERV-K protease and, the HIV-1 protease was used for the rest of the model. Three features common to retroviral proteases were carefully maintained in the alignment and model: 1) the active site triad (26-DTG-28), 2) the highly conserved triad GRN/D unique to retroviral proteases [24], and 3) the intra- and inter-subunit salt bridge between R89, D30, and R9′. To model complexes of HERV-K protease with inhibitors the structures of the highest resolution complexes of the HIV-1 protease with Lopinavir (2OS4), and Darunavir (2HS1) were used. Models of the complexes with Darunavir and Lopinavir were prepared by overlaying the respective complexes structures with the HERV-K model.

In the case of the HERV-K reverse transcriptase, HIV-1 RT crystal structure (4W1E) was used as template, and the crystal structures of the complexes with Efirvarenz (1JKH), Nevirapine (3QIP), and Etravirine (3MEC) were used to model the inhibitors bound to HERV-K RT.

In the case of the HERV-K integrase the simian prototypical foamy virus in complex with magnesium, DNA, and Elvitegravir (3L2U) with an 18% of identity with the target was used as template. This elvitegravir complex and the complexes with ratelgravir (3L2V) and dolutegravir (3S3M) were used to model these inhibitors complexes with HERV-K integrase.

### Example 1: HERV-K is Expressed in the Brain Tissue of ALS Patients

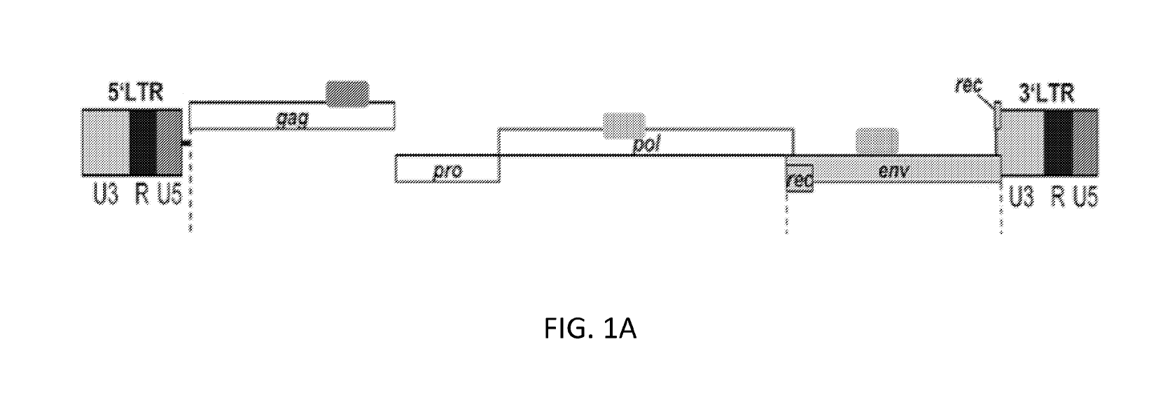

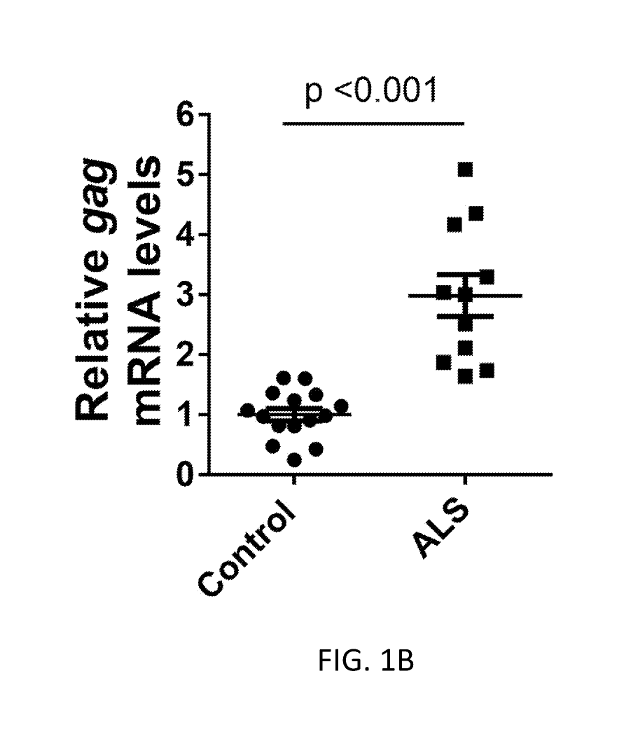

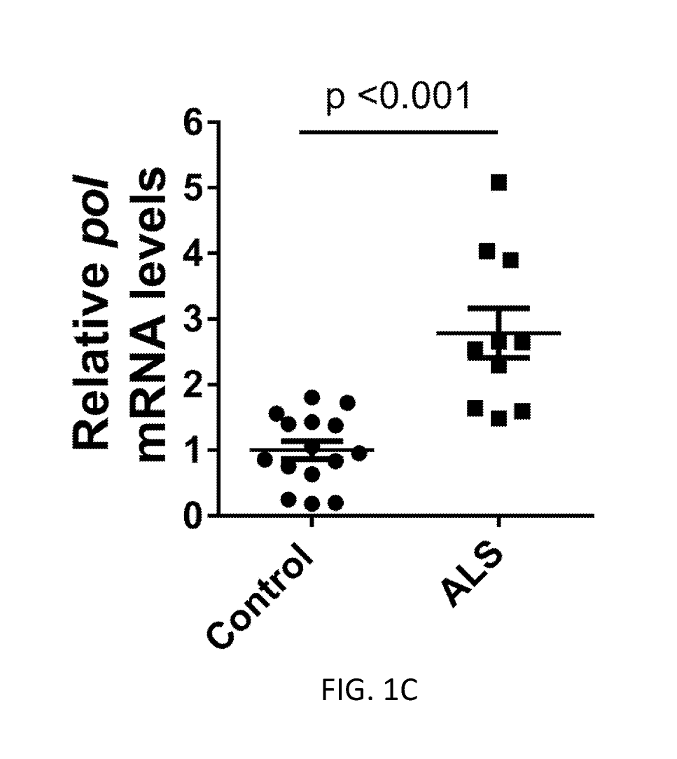

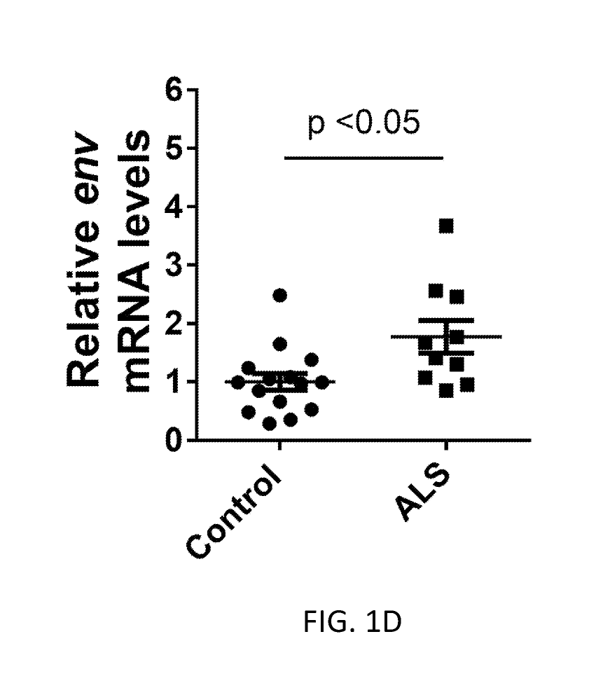

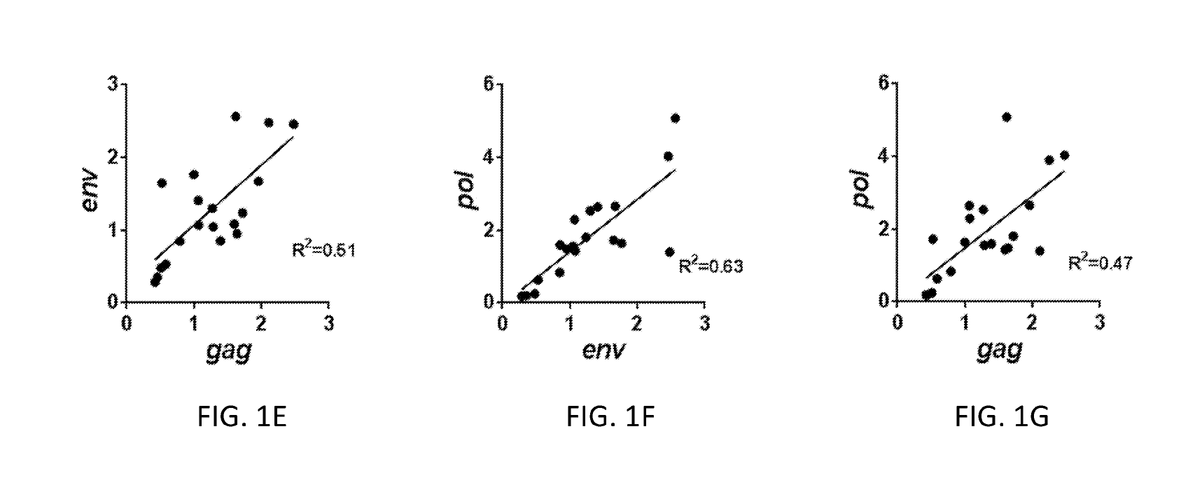

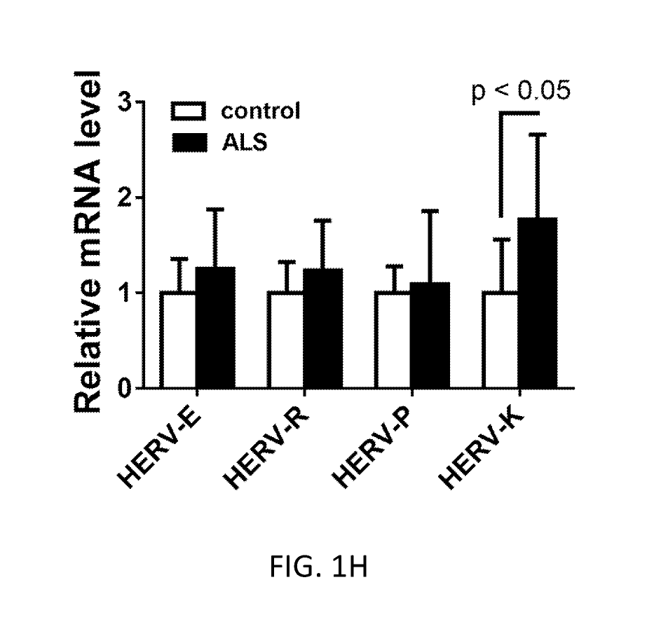

The HERV-K genome, similar to that of other retroviruses has three major structural genes, the gag, pol and env genes that encode the capsid, reverse transcriptase and envelope proteins, respectively. Primer sets were used to amplify transcripts from each of these genes by reverse transcriptase polymerase chain reaction (RT-PCR) (FIG. 1A). The inventors found that transcripts for all three genes were elevated in postmortem brain tissue samples from ALS patients (FIGS. 1B-1D). There was good correlation between the expression of each of these genes (FIGS. 1E-1G) confirming that the entire viral genome was expressed in these patient samples. The expression of HERV-K was also compared to the expression of several other HERVs. No significant elevation of these HERVs was noted (FIG. 1H). Given that there are multiple loci that encode the HERV-K genome, the inventors conducted RNA sequencing and analyzed the transcripts of each of the loci. The loci at chromosomes 7C and 10A were expressed in all three postmortem brain samples from patients with sporadic ALS at higher levels compared to controls. No specific clinical phenotype was associated with the expression of HERV-K in ALS patients. To determine the cell types in which HERV-K was expressed, the inventors immunostained postmortem brain tissue from patients with ALS and found expression of the env protein within the cortex of 3/5 individuals with strong expression in the cytoplasm of large pyramidal neurons. Anterior horn neurons in the spinal cord also showed a similar pattern of immunostaining for HERV-K env protein. No immunostaining was seen in the lateral or posterior horns of the spinal cord. No immunostaining was noted in the glial cells or in the white matter. Furthermore, no immunostaining was noted in cortex or white matter of brain tissue from healthy individuals or in postmortem brain tissue from patients with Alzheimer's disease. However, robust immunostaining for amyloid was present in postmortem brain tissue from Alzheimer's disease patients.

### Example 2: Expression of HERV-K in Human Neurons In Vitro Causes Toxicity

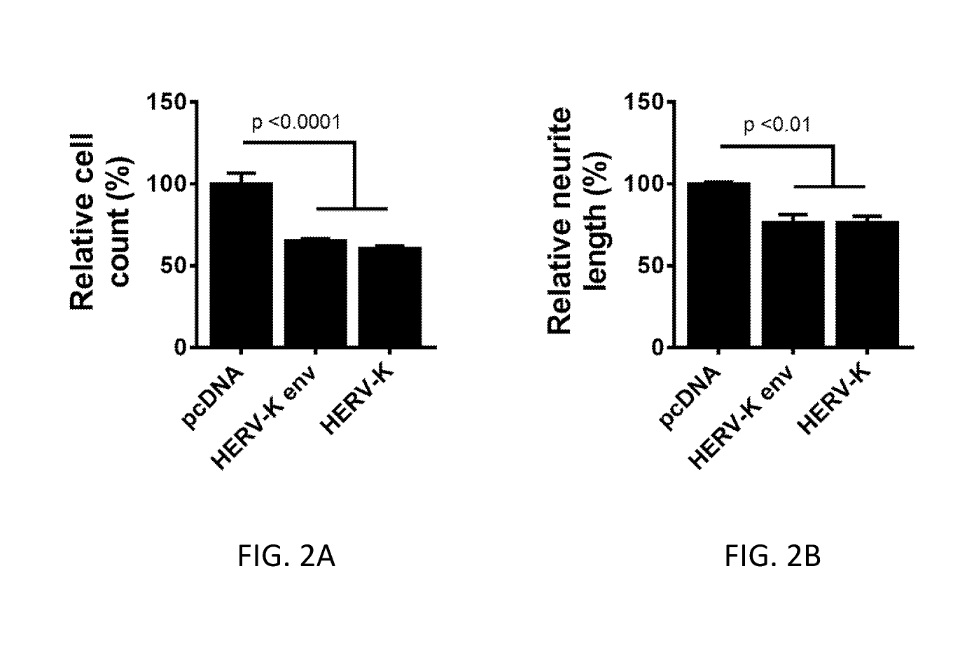

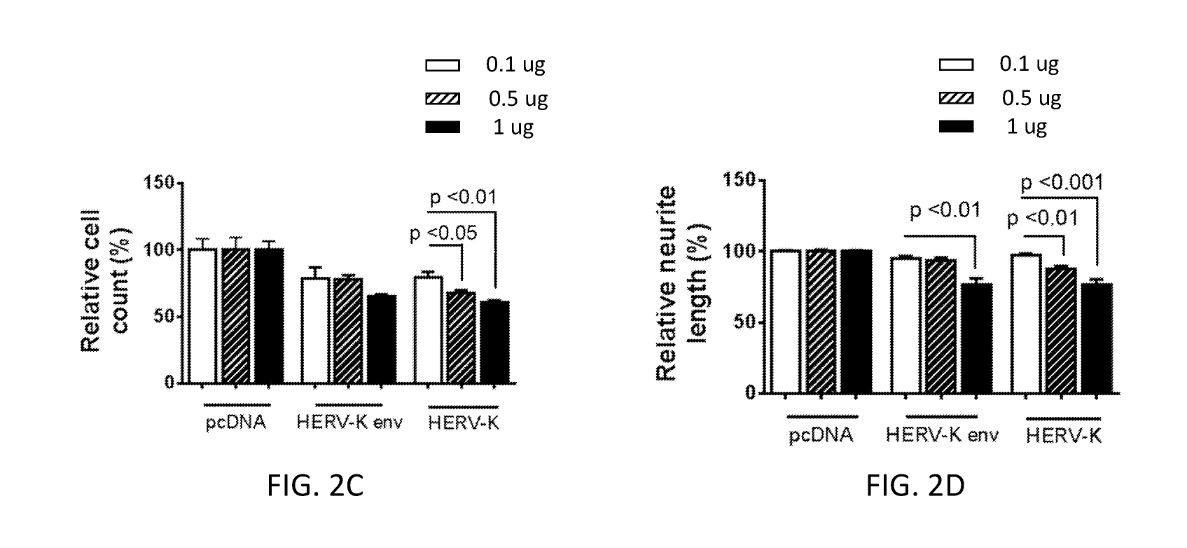

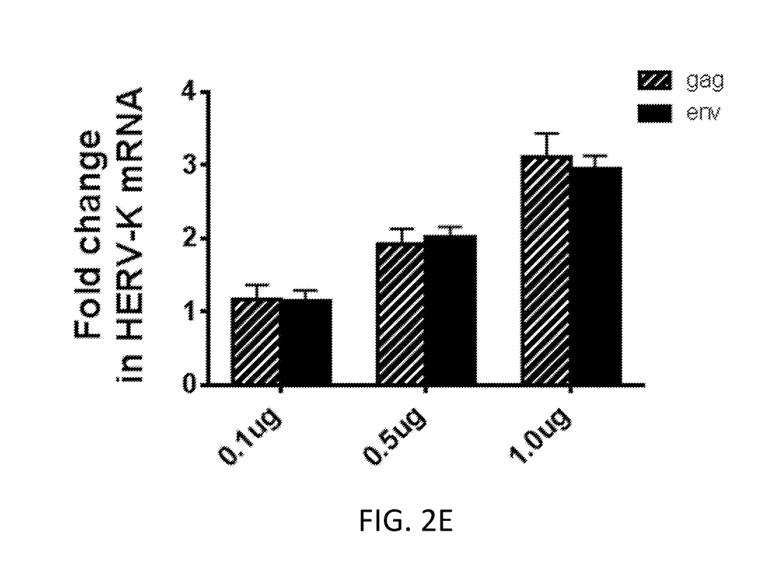

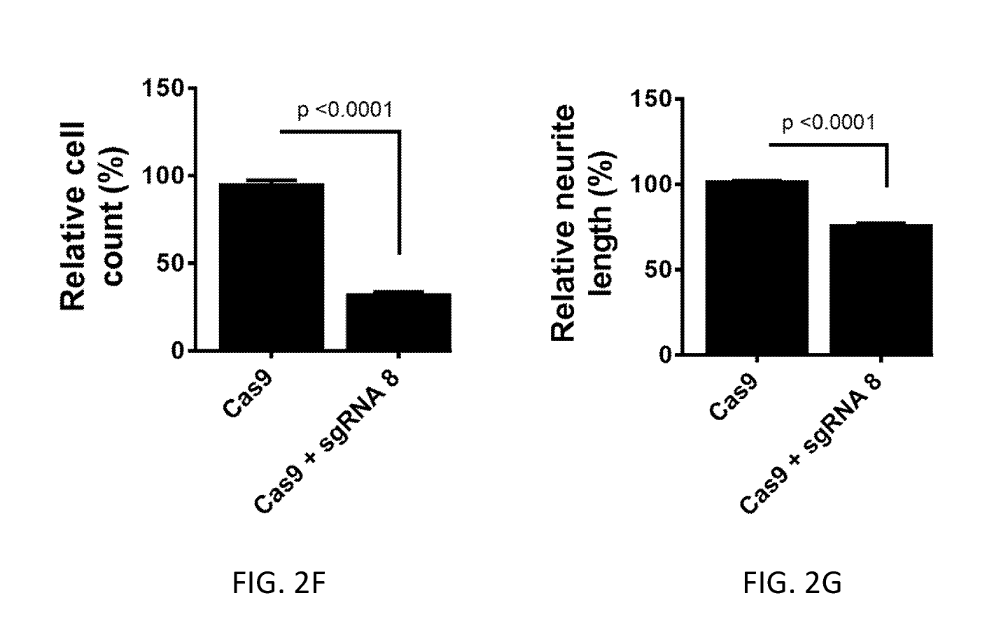

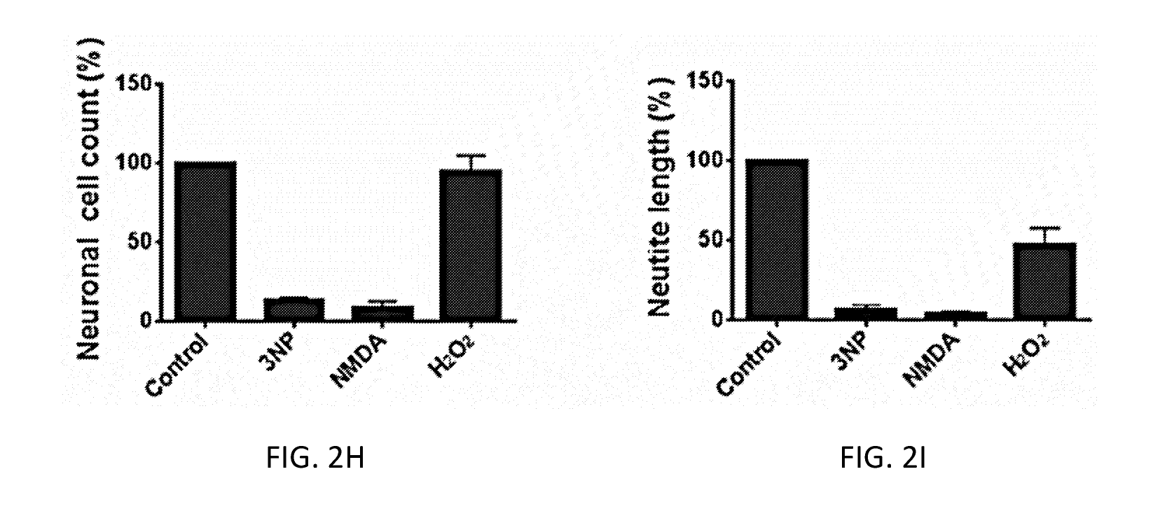

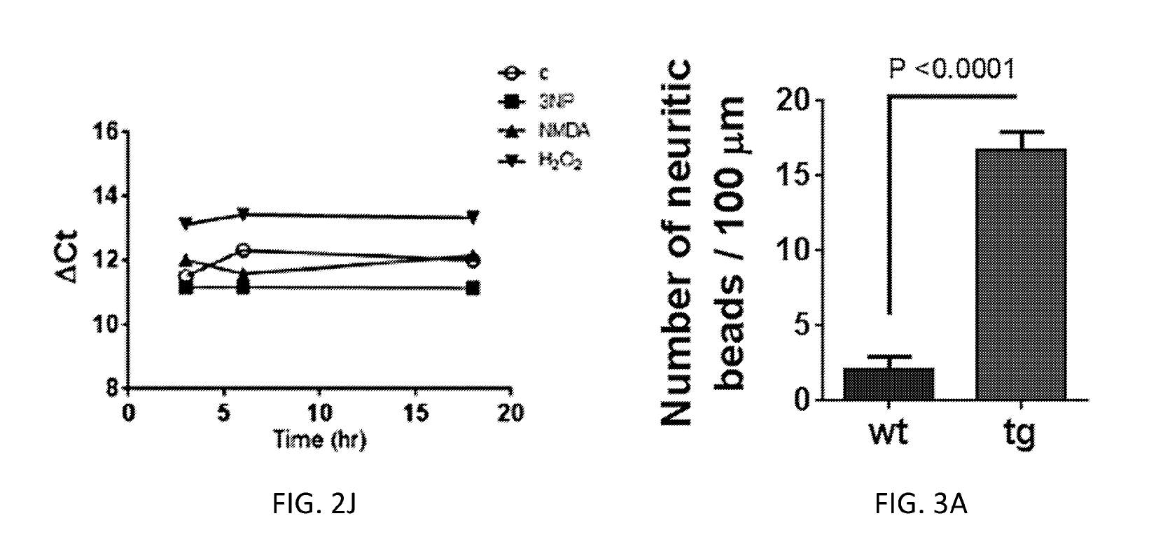

To determine the relevance of HERV-K expression in neurons, the inventors transfected the HERV-K genome and the HERV-K env gene into human neuronal cultures. Both the entire HERV-K genome and the env gene caused a similar decrease in cell numbers and retraction of neurites (FIGS. 2A and 2B) in a dose-dependent manner (FIGS. 2C and 2D). The expression of the genes was confirmed by RT-PCR (FIG. 2E). This suggested that the env protein could contribute to neurotoxicity and neuronal death. To determine the effect of activation of endogenous HERV-K in neurons, the inventors used a gene editing tool CRISPR/Cas9 in which the nuclease activity of Cas9 was altered to contain four copies of the transcription factor VP16. This was delivered to the human neurons in culture via a lentiviral vector. The inventors designed a guide RNA (sgRNA8) to direct the transcription factor to the LTR region of HERV-K. Activation of the endogenous HERV-K via the LTR resulted in neurotoxicity as evidenced by loss of neurons (FIG. 2F) and retraction of neurites (FIG. 2G). Activation of the viral genes was confirmed by RT-PCR and a two-fold increase in expression above controls was observed. To determine if the process of neuronal injury led to HERV-K activation, the inventors treated the neurons with 3-nitropropionic acid, N-methyl-D-aspartate or hydrogen peroxide. No activation of HERV-K was noted as determined by measuring viral transcripts (FIG. 2H-2J).

### Example 3: Expression of HERV-K Env In Vivo Causes Degeneration of Motor Neurons

The findings were initially confirmed in vivo by in utero electroporation of the env gene into embryonic mouse brain which resulted in dysmorphic changes in neurons and punctuate dilatation of neuronal processes (FIG. 3A). The inventors next generated transgenic animals in which the env gene was expressed in neurons. Expression of transcripts in the animals was confirmed by RT-PCR. The gene produced the full length and transmembrane domain of the env protein. The level of activation of HERV-K env transcripts was nearly two-fold higher in the transgenic animals compared to postmortem brain tissue of patients with ALS (FIG. 3B). HERV-K env protein was detected by immunostaining and showed widespread expression in cortical neurons of transgenic mouse brain. Similar to ALS patient tissues, the mouse neurons showed expression of the env protein in the neuronal cell bodies within the cytoplasm and the apical dendrites. There was accompanying astrocytosis in regions surrounding the neurons where HERV-K env was expressed but there was no difference in immune reactivity of microglial cells. Golgi staining showed decreased length, branching and complexity of dendrites (FIG. 3C-3E). The number of dendritic spines was also decreased (FIG. 3F) and was associated with morphological changes showing loss of stalks resulting in an increase in stubby spines and a decrease in mushroom spines (FIGS. 3G and 3H). There was also beading of the axons and dendrites.

### Example 4: HERV-K Env Transgenic Animals Display Specific Loss of Upper and Lower Motor Neurons

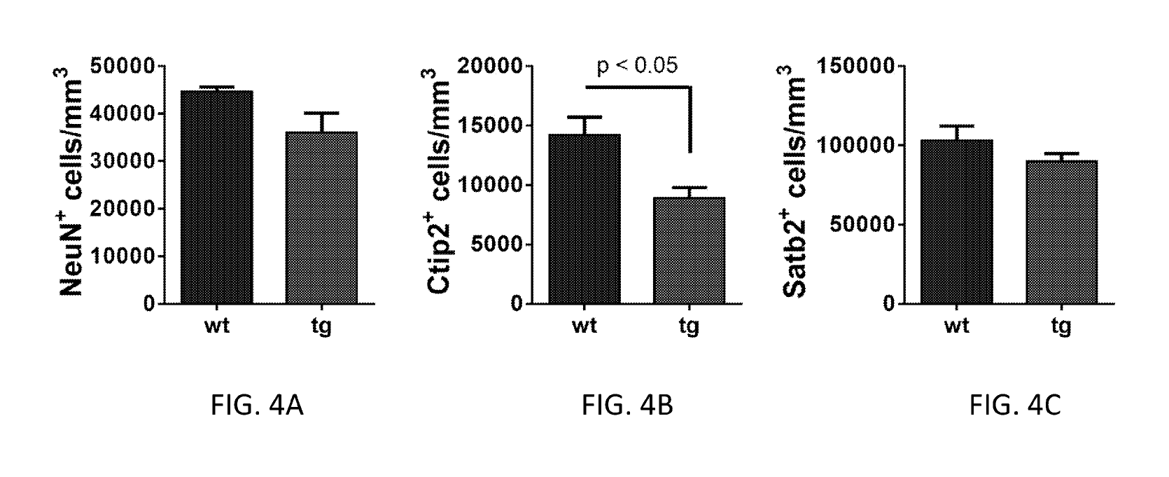

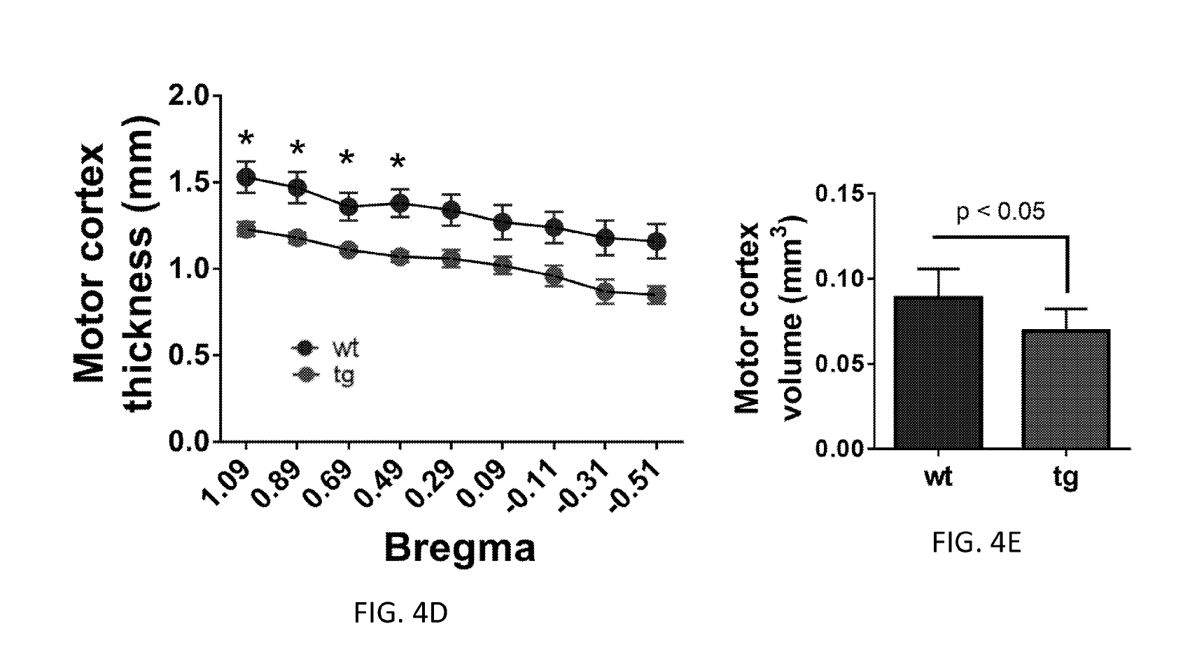

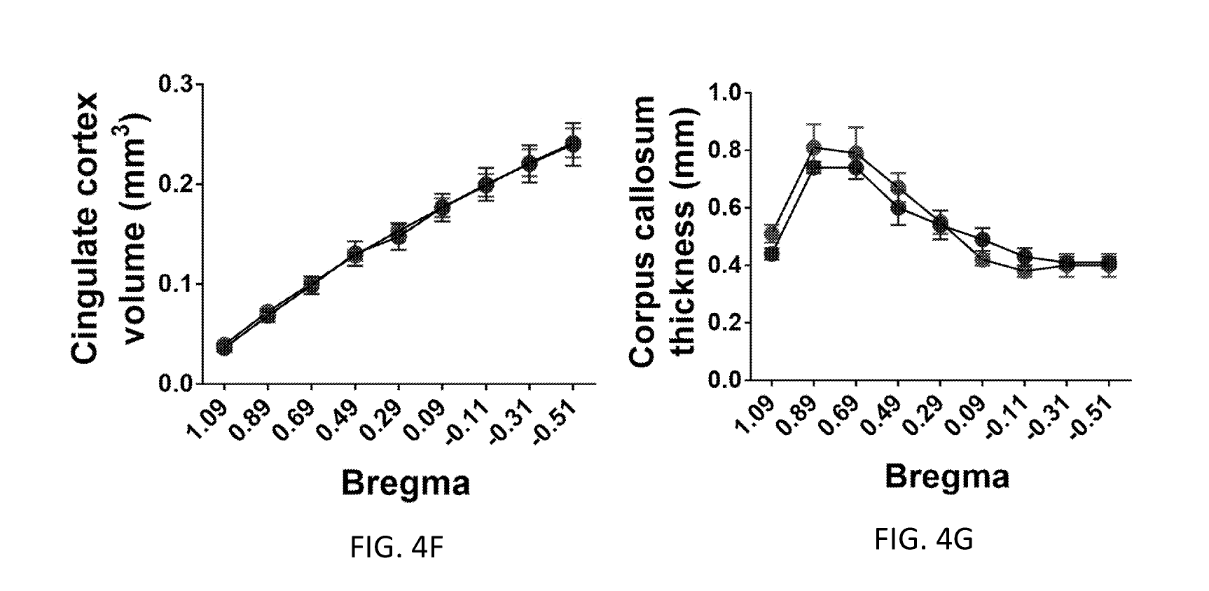

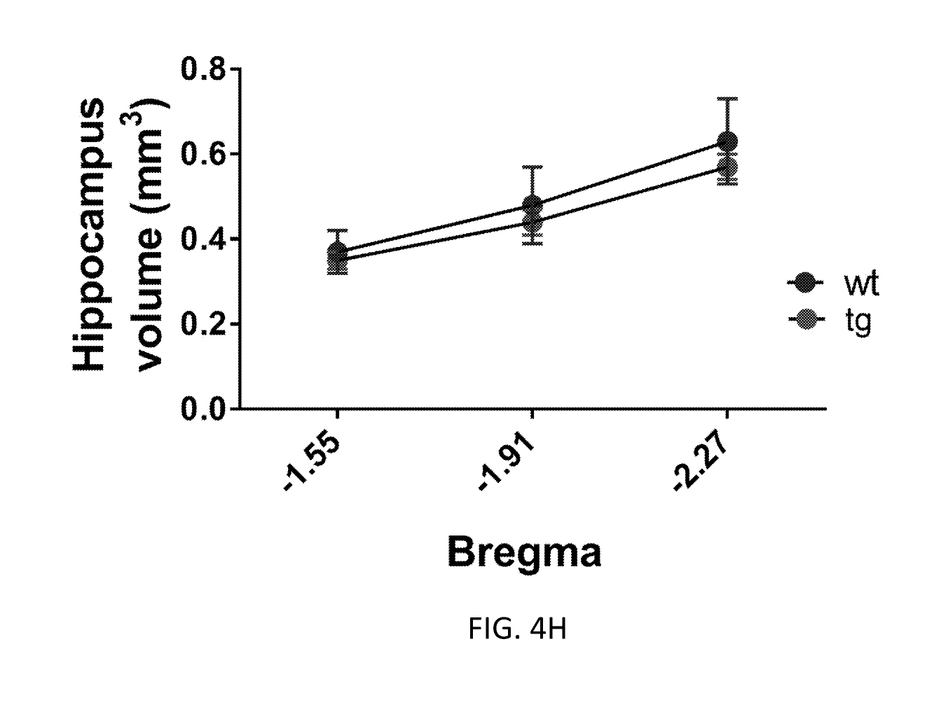

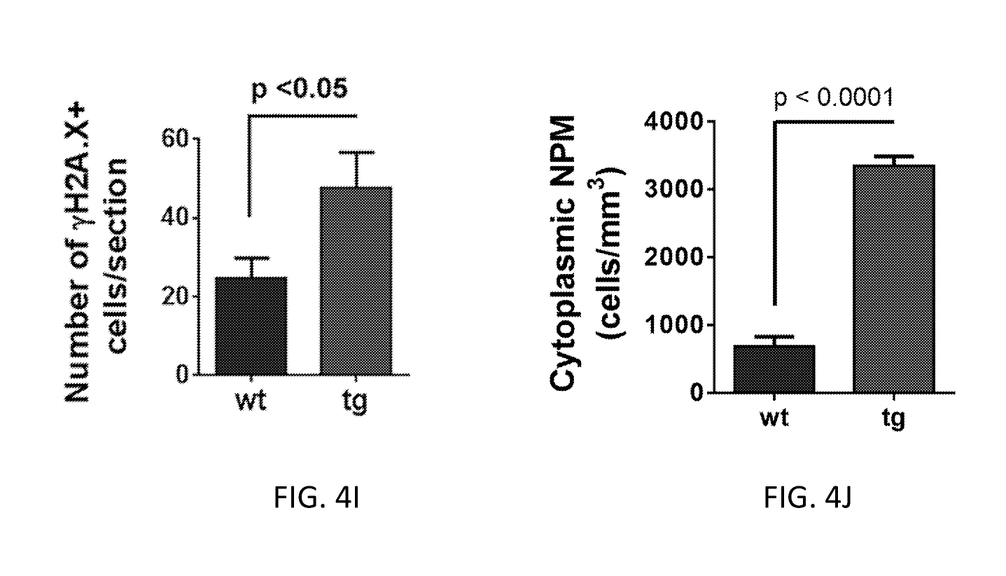

Immunostaining for neurons expressing NeuN showed no significant change in numbers of neurons in the frontal cortex (FIG. 4A). However, corticospinal motor neurons immunostained for Ctip2 showed a decrease in cell numbers (FIG. 4B; p<0.05). In contrast, there was no significant change in the number of callosal projection neurons staining positive for Satb2, suggesting that the effect was specific for motor neurons (FIG. 4C). MR images of the brain of transgenic animals also showed a specific decrease in thickness and volume of the motor cortex (˜22%) with no significant changes in the volume of the cingulate cortex and hippocampus or the thickness of the corpus callosum (FIG. 4D-4H) or the architecture of the brain. Immunostaining of the transgenic mouse spinal cord showed widespread expression of HERV-K env in neurons. However, only rare motor neurons were present in the anterior horns with near absence of motor neurons at some levels of the spinal cord. Immunostaining of the quadriceps and tibialis anterior muscles for type I and type II myosin isoforms showed fiber type grouping and examples of grouped atrophy suggestive of a chronic denervation and reinnervation process. There were no dystrophic changes in the muscle fibers, and the nuclei were in the periphery of the fibers suggesting that there were no myopathic features.

Ongoing neuronal injury was also evident by the presence of double-stranded DNA breaks as seen by immunostaining for γH2A.X, which showed aggregated foci of the phosphorylated histone protein within the chromatin. Nucleolar dysfunction has been observed in several neurodegenerative diseases including Alzheimer's disease and Parkinson's disease. The inventors therefore evaluated whether neurons from transgenic mice showed signs of nucleolar stress. The number of γH2A.X foci was increased in neurons in the frontal cortex (FIG. 4I). Immunostaining for the nucleolar marker, nucleophosmin, showed translocation from the nucleolus to the cytoplasm of cortical neurons (FIG. 4J). Together these data suggested that disruption of nucleolar function may be a key mechanism by which HERV-K leads to neuronal dysfunction.

### Example 5: HERV-K Env Transgenic Animals Develop Motor Dysfunction

To determine the functional consequences of HERV-K env expression in neurons, the inventors performed a panel of behavioral tests on the animals. These tests showed that the animals developed progressive motor dysfunction. In an open field they traveled shorter distances and rested for longer periods of time (FIG. 5A to 5E). Transgenic mice fell faster in a rotarod performance test (FIG. 5F) and displayed evidence of spasticity with increased clasping of the hind limbs (FIG. 5G). Y maze testing confirmed that these differences were not due to an impairment of working memory (FIG. 5H). Sensory and vestibular functions were also unimpaired (FIGS. 5I and 5J). Motor function in the transgenic mice also showed a progressive decline from 3 to 6 months of age as evaluated in open field testing with 50% mortality by 10 months (FIG. 5K). In terminal stages, the animals developed profound weakness of the limbs and spinal muscles resulting in minimal movement and a hunched back causing decreased movement of the thoracic cage affecting the muscles of respiration (FIG. 5K).

Functional activity of the neurons was also assessed in electrophysiological recordings. Passive and active membrane properties of layer V cortical pyramidal neurons from wildtype and transgenic mouse prefrontal cortices were tested by injecting gradient steps of electrical current into the cell. Subthreshold responses (current steps from −120 pA to 20 pA) reflected an increase in the global input resistance of the cell, as shown in the following table:

Passive and active membrane properties of mPFC L5 pyramidal neurons.
RMP, resting membrane potential; AHP, after hyperpolarization; parameters of the first spike were measured during a current ramp 200 pA/s.

| Variables | wt | tg | p values |
|---|---|---|---|
| Number of neurons | 15 | 17 | |
| Passive membrane | | | |
| properties | | | |
| RMP (mV) | 68.7 ± 0.6 | 65.4 ± 1.2 | ns |
| Input resistance (MΩ) | 140.2 ± 20.2 | 219.8 ± 27.7 | * |
| Active membrane | | | |
| properties | | | |
| Rheobase (pA) | 71.3 ± 7.9 | 51.4 ± 8.9 | * |
| Threshold (mV) | −43.3 ± 0.9 | −42.8 ± 1.1 | ns |
| Amplitude (mV) | 82.5 ± 2.8 | 78.4 ± 2.1 | ns |
| Rise time 10-90% (ms) | 0.34 ± 0.02 | 0.36 ± 0.01 | ns |
| Latency (ms) | 456.3 ± 47.3 | 316.8 ± 41.2 | * |
| Width (mV) | 0.98 ± 0.05 | 0.98 ± 0.02 | ns |
| AHP amplitude (mV) | −10.4 ± 0.5 | −10.0 ± 0.6 | ns |

Membrane excitability was assessed with a series of depolarizing current steps to evoke action potentials. The inventors found that the number of action potentials was enhanced in transgenic animals resulting in a right shift of the input-output function curve. This increase in intrinsic excitability is associated with a decrease in the first action potential latency and the rheobase, defined as the minimal current to induce an action potential. Other action potential parameters like threshold, amplitude, rise time width and after hyperpolarization amplitude remained unchanged. Finally, the inventors examined synaptic transmission by recording spontaneous excitatory and inhibitory postsynaptic currents (sEPSCs and sIPSCs). The inventors found that only the sEPSC amplitude was changed, with a significant decrease in sEPSC observed in transgenic animals compared to wildtype mice, which could be attributed to the decrease in spine density (FIG. 5L-5Q). The increase in input resistance was consistent with the decrease in neurite number and branching.

### Example 6: HERV-K Expression is Regulated by TAR DNA-Binding Protein 43

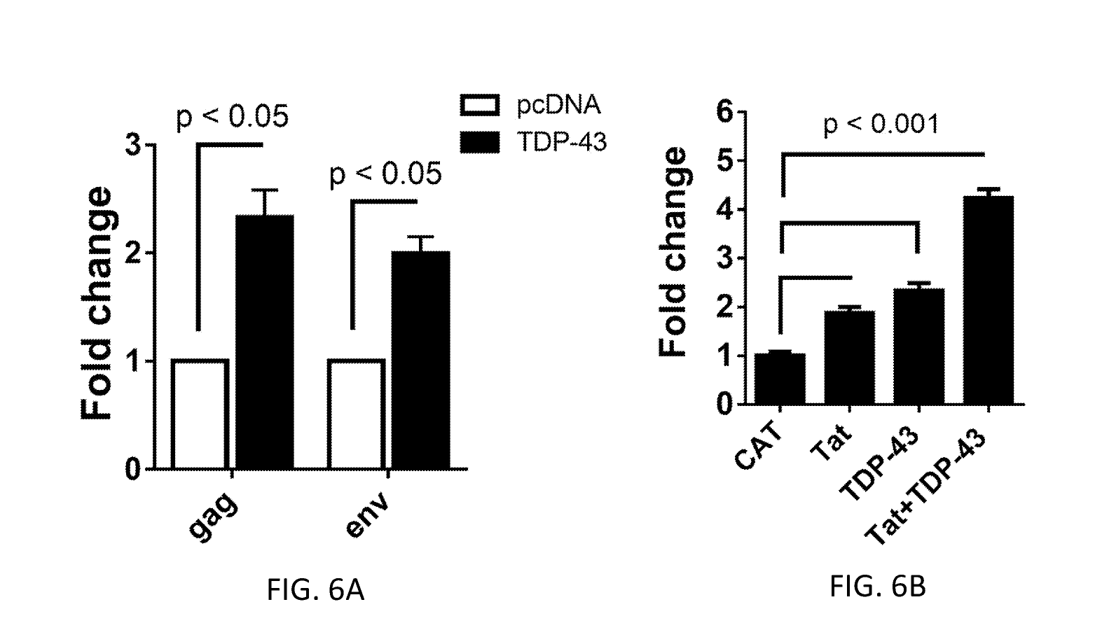

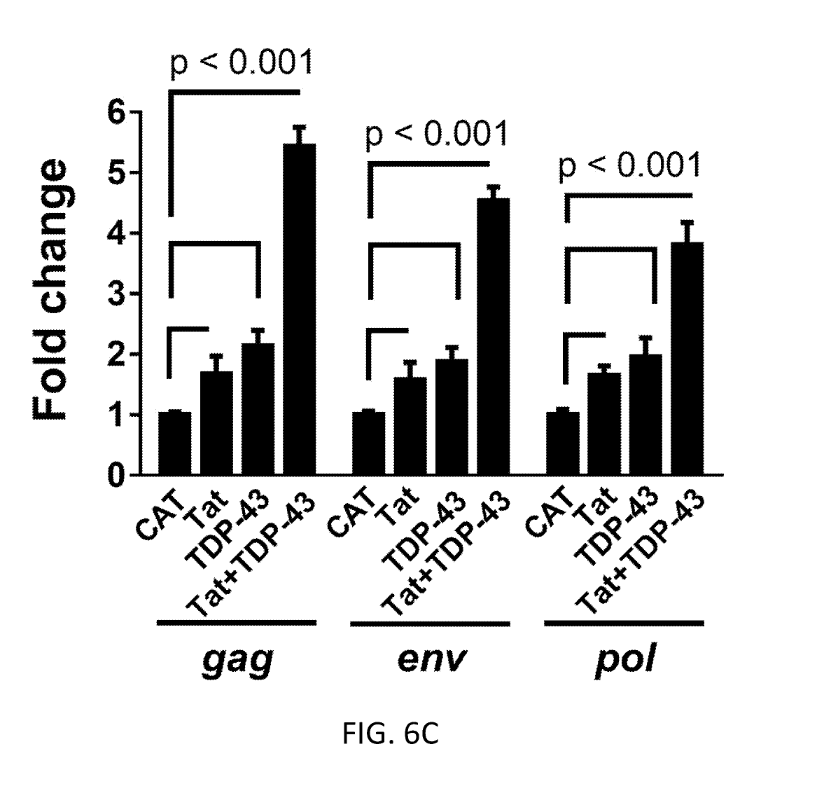

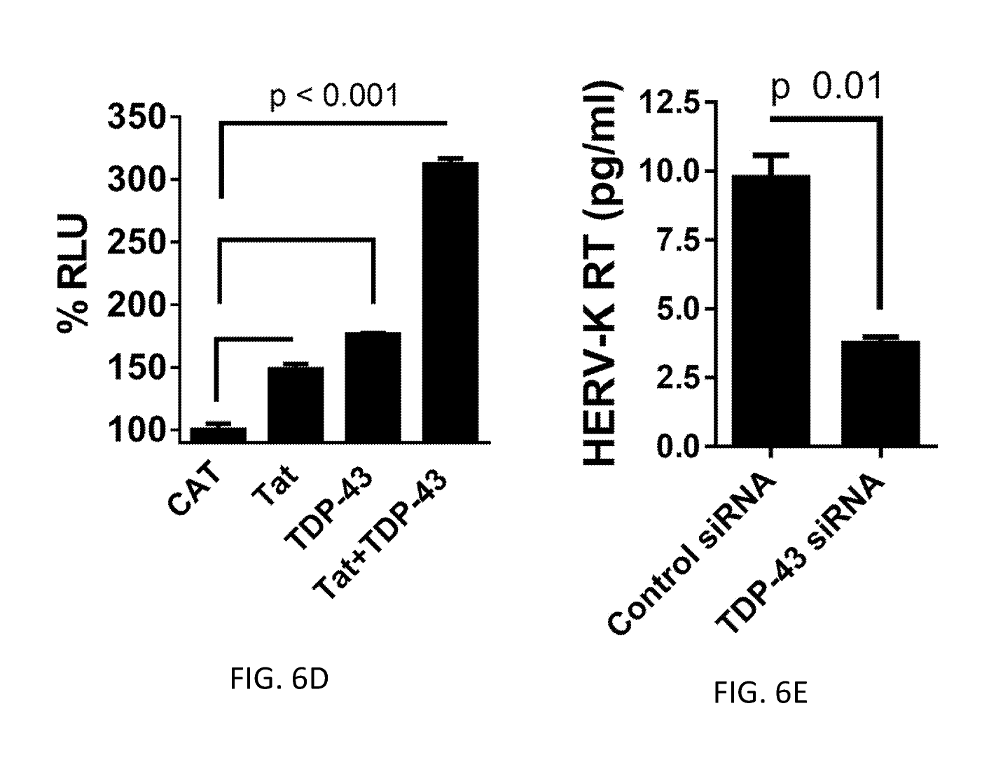

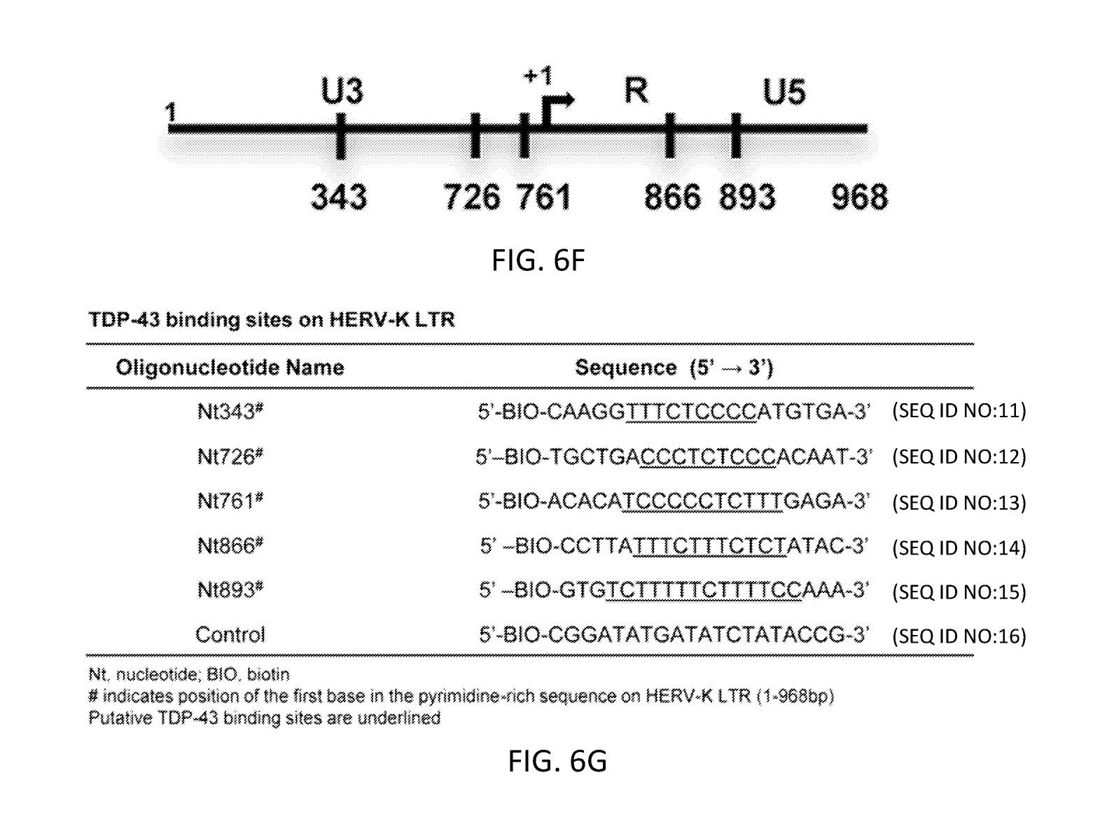

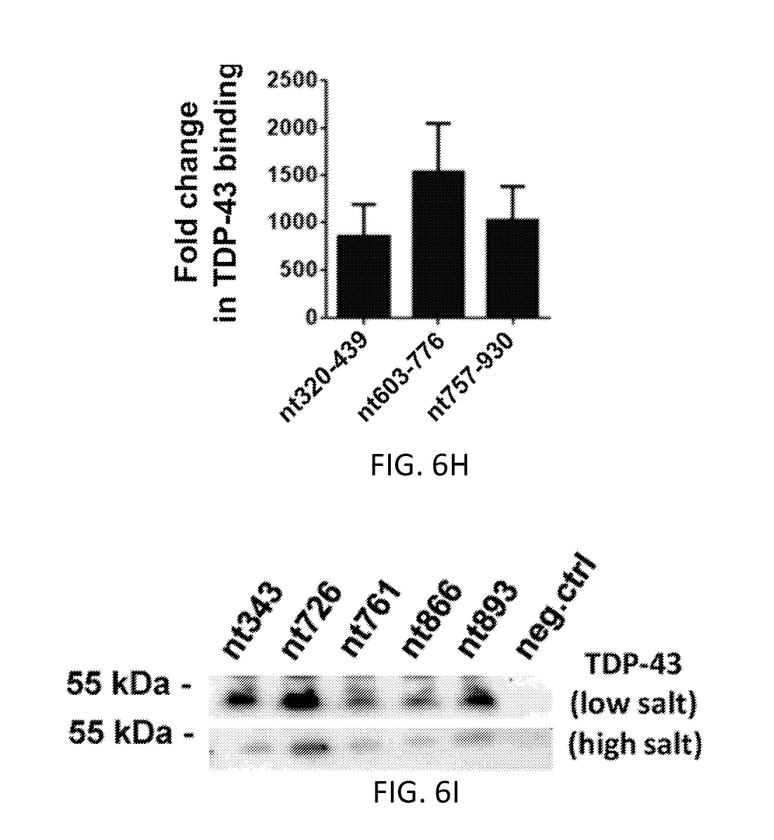

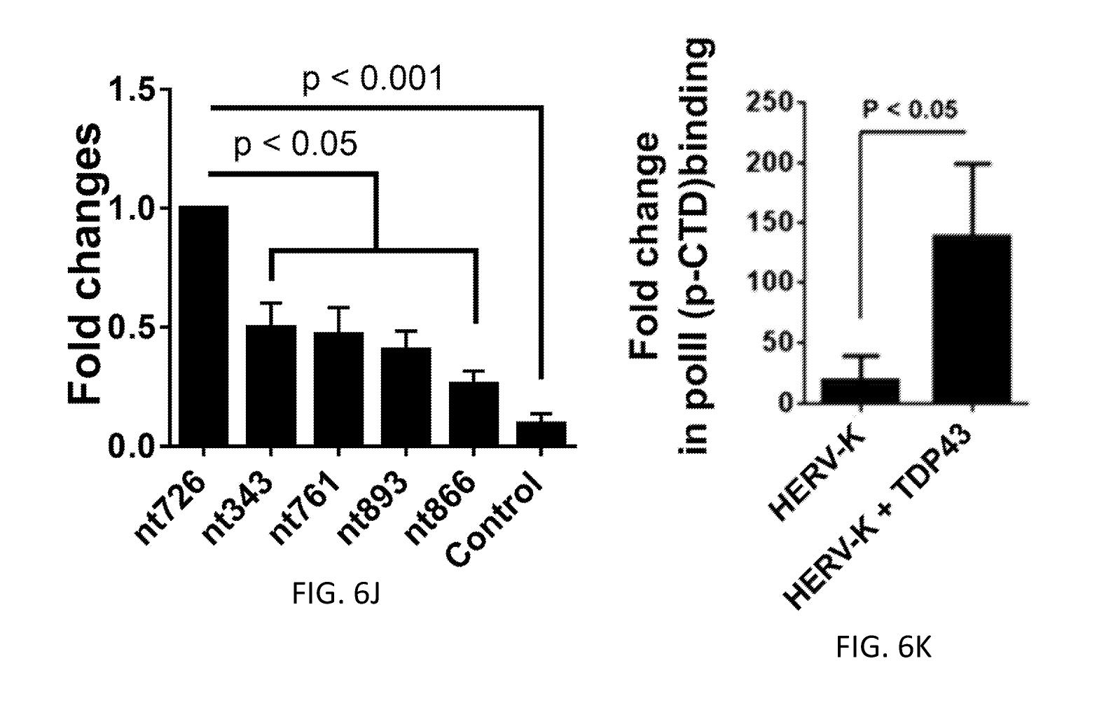

Previously, HERV-K pol gene expression was found to correlate with TAR DNA-binding protein 43 (TDP-43) mRNA in postmortem brain tissue from patients with ALS. TDP-43 has been shown to regulate the replication of the human immunodeficiency virus (HIV) and it also binds to transposable elements. Hence, the inventors determined whether TDP-43 could also regulate HERV-K expression. When a plasmid with TDP-43 was transfected into human neurons, HERV-K expression occurred as demonstrated by immunostaining for the env protein and measuring the viral transcripts (FIG. 6A). When HERV-K and TDP-43 were co-transfected into HeLa cells, there was increased replication of HERV-K as evidenced by reverse transcriptase activity in the culture supernatants (FIG. 6B) and increased viral transcripts in the cell extracts (FIG. 6C). HIV-Tat protein is known to increase HERV-K replication hence it was used as a control. TDP-43 showed additive responses with Tat suggesting that they may act on different sites. This was confirmed using a HERV-K LTR construct with a luciferase reporter gene (FIG. 6D). Knockdown of endogenous TDP-43 with siRNA also decreased HERV-K expression (FIG. 6E). The inventors next determined whether TDP-43-mediated induction of HERV-K involved direct association with the HERV-K promoter. The consensus HERV-K LTR sequence was scanned to identify pyrimidine-rich motifs associated with TDP-43 DNA binding. Putative TDP-43 binding sites, consisting of contiguous pyrimidine bases, were identified at five loci as indicated, and labeled according to their position relative to the first base of the HERV-K LTR (FIG. 6F; FIG. 6G). Binding of TDP-43 to HERV-K LTR was confirmed by chromatin immunoprecipitation (ChIP) (FIG. 6H). Hence, the inventors constructed biotinylated oligomers representing each of these sites and incubated them with nuclear extracts from 293T cells followed by washing of DNA/protein complexes under low and high salt conditions and analyzed the complexes by Western blots using an antibody to TDP-43. The inventors found that TDP-43 bound to region 726-734 (5′-CCCTCTCCC-3′) with highest affinity, suggesting that it was the critical binding site on the HERV-K LTR (FIGS. 6I and 6J). TDP-43 binding to HERV-K LTR was associated with increased binding of elongation-competent RNA polymerase 11 (FIG. 6K). No effect was seen on an unrelated genomic region.

These data demonstrate that the HERV-K virus was expressed in cortical and spinal neurons in ALS patients, but not control healthy individuals. Expression of HERV-K or its env protein in human neurons caused retraction and beading of neurites. Transgenic animals expressing the env gene developed progressive motor dysfunction accompanied by selective loss of volume of the motor cortex, decreased synaptic activity in pyramidal neurons, dendritic spine abnormalities, nucleolar dysfunction and DNA damage. Injury to anterior horn cells in the spinal cord was manifested by muscle atrophy and pathological changes consistent with nerve fiber denervation and reinnervation. Expression of HERV-K was regulated by TAR DNA-binding protein 43 which binds to the long terminal repeat region of the virus. Thus, HERV-K expression within neurons of patients with ALS likely contributes to neurodegeneration and disease pathogenesis.

### Example 7: Testing the Effect of Nucleoside and Non-Nucleotide Reverse Transcriptase (RT) Inhibitor Drugs on HERV-K Reverse Transcriptase

Human Endogenous Retroviruses (HERVs) are genomic sequences of retroviral origin that account for nearly 8% of the human genome. Although mostly defective and inactive, some of the HERVs may be activated under certain physiological and pathological conditions. While no drugs are designed specifically to target HERVs, antiretroviral drugs are designed against the human immunodeficiency virus. To determine if the antiretroviral drugs have an effect on HERV-K replication, a plasmid was constructed with consensus HERV-K sequence that produced HERV-K virus, and used to show that all reverse transcriptase (RT) inhibitors could significantly inhibit HERV-K reverse transcriptase activity.

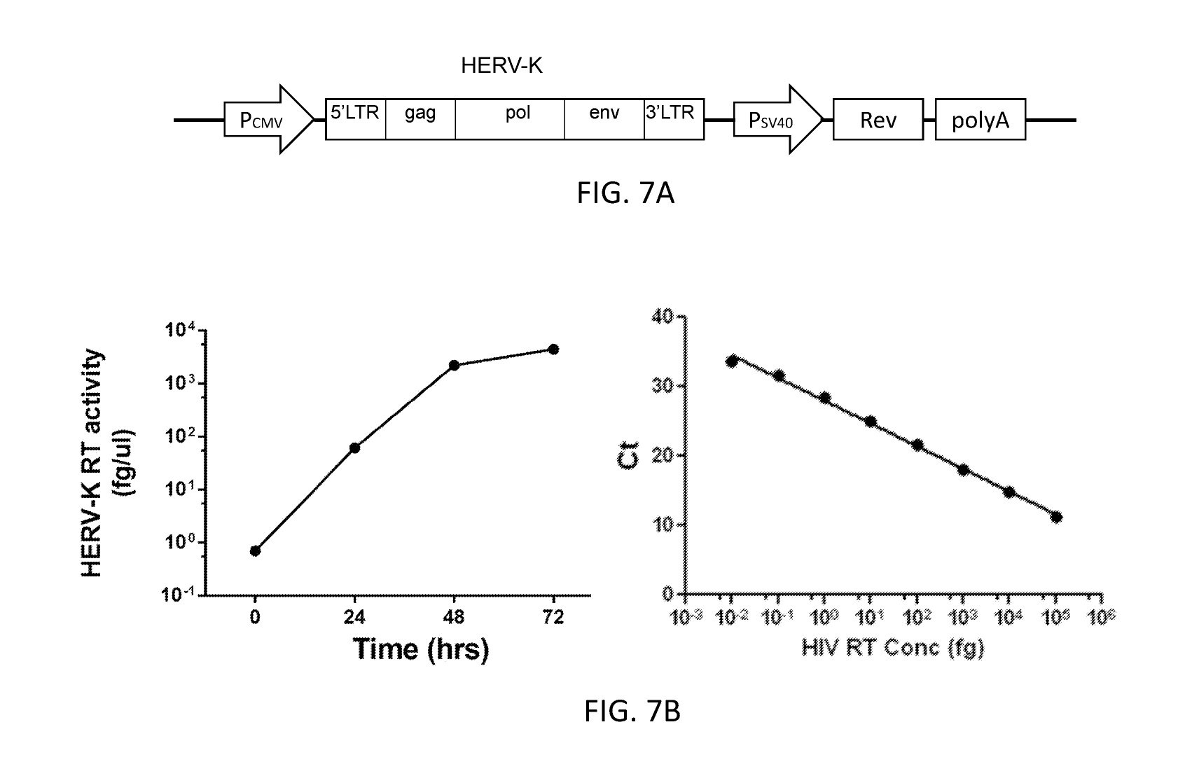

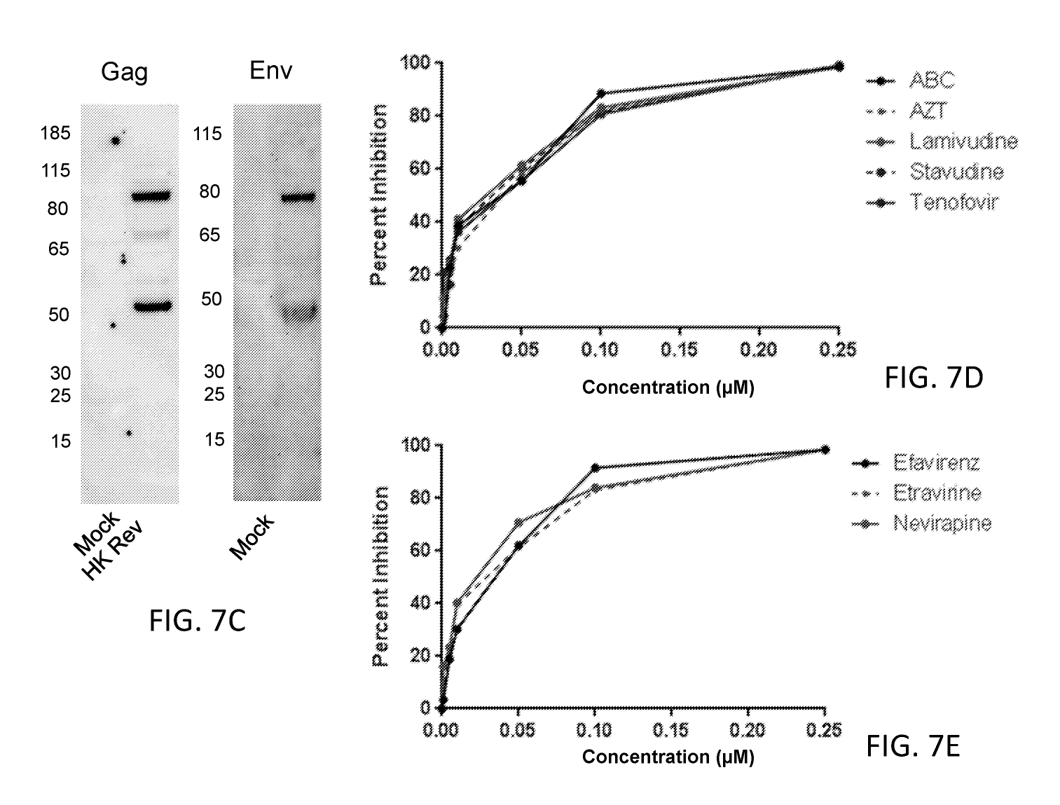

To generate HERV-K viral particles for determining the effects of antiretroviral drugs on HERV-K replication, a consensus HERV-K sequence was synthesized and cloned into pcDNA 3.1 vector. Because HIV-1 Rev can significantly enhance the transcription of HERV-K viral gene, an HIV-1 Rev expression cassette was also inserted into the construct which (“pCD-HK/Rev”; FIG. 7A). After transfection of either HeLa or 293T cells with the pCD-HK/Rev plasmid, the production of viral particles was confirmed by electron microscopy. The amount of viral production was quantified by measuring reverse transcriptase (RT) activity in the culture supernatant with PERT assay (FIG. 7B, left). Recombinant HIV-1 RT was used as a positive control and to make a standard curve (FIG. 7B, right). HERV-K viral particles released to the culture media increased until 48 hr after transfection, and plateaued at 72 hr. To further confirm HERV-K viral gene expression, cell lysate was collected 48 hr post-transfection. The expression of HERV-K Gag and Env was determined by Western blot analysis (FIG. 7C). The Gag antibody recognized both precursor Gag (90 kD) and mature Gag (50 kD) proteins. The Env antibody recognized both full-length (90 kD) Env and the transmembrane subunit (42 kD). HERV-K Gag and Pol were immunostained in HERV-K plasmid transfected 293T cells. Some cells co-expressed HERV-K Gag and Pol while other cells expressed only Gag or Pol.

The effects of HIV-1 RT inhibitors on HERV-K RT enzyme activity was first transfected in a cell-free system. Viral particles were harvested from either HeLa or 293T culture media after transfection with HERV-K plasmid. HERV-K RT was then released from culture media by treatment with Triton X-100. PERT assay was used to determine the activity of HERV-K RT. Serial dilutions of inhibitors were added to the extracted HERV-K RT just prior to the PERT assay. Tenofovir, Abacavir, Stavudine, Lamivudine, and Zidovudine nucleotide RT inhibitors were tested. As shown in FIG. 7D, all nucleotide RT inhibitors showed significant and dose-dependent inhibition of HERV-K RT. They had similar dosage-response curves and 1090 values. The non-nucleotide inhibitors Efirvarenz, Etravirine, and Nevirapine were also tested. These drugs also showed significant inhibition of HERV-K RT activity with similar 1090 values (FIG. 7E).

### Example 8: Testing the Effects of Antiretroviral Drugs on HERV-K Viral Replication in HeLa Cells

HERV-K was pseudotyped with VSV-G and used to infect HeLa cells. Replication of HERV-K was measured by quantitative real time polymerase chain reaction (qRT-PCR). We found that RT inhibitors Abacavir and Zidovudine, and integrase inhibitor Raltegravir can effectively block the replication of HERV-K. However, protease inhibitors were not as effective as RT and integrase inhibitors.

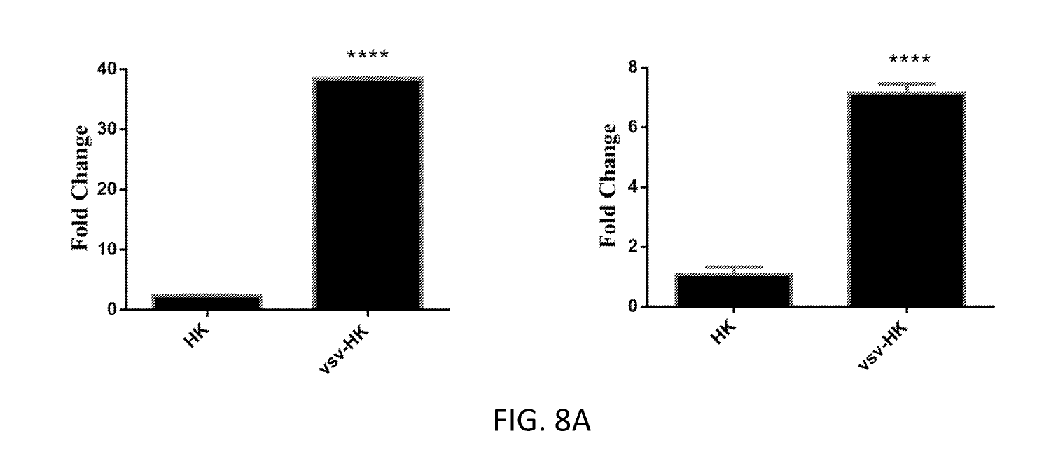

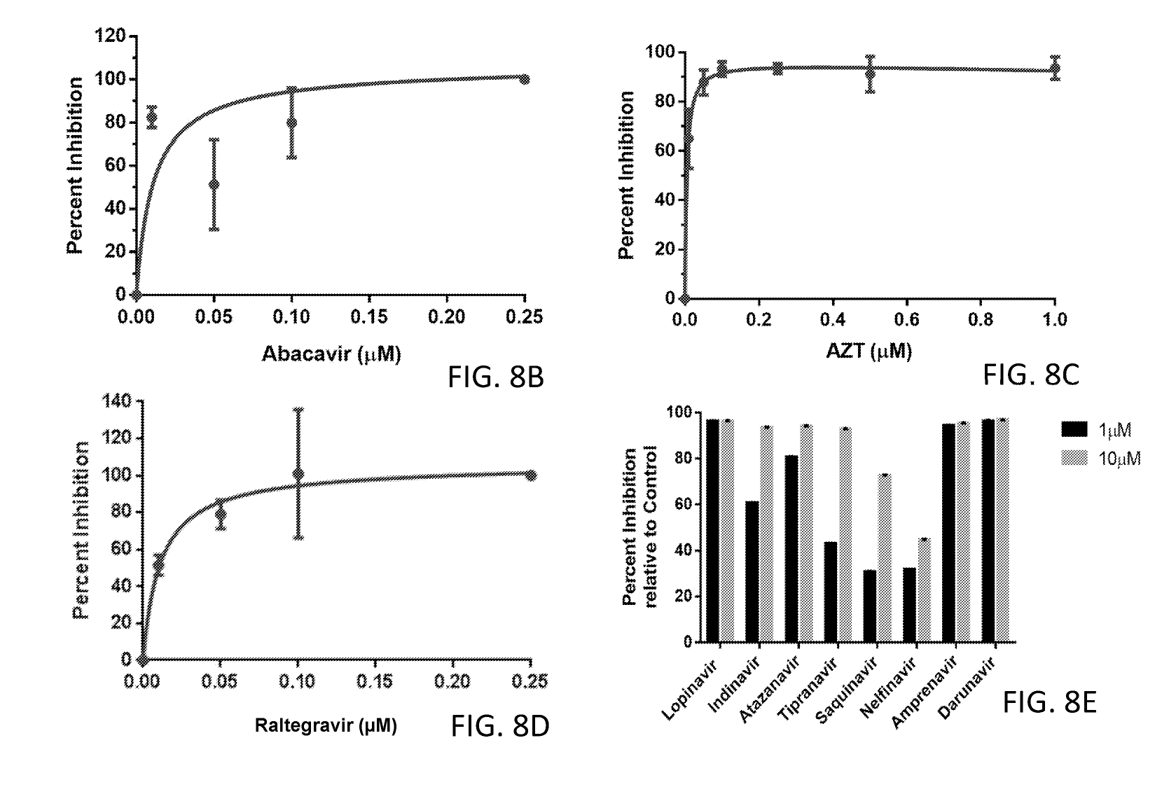

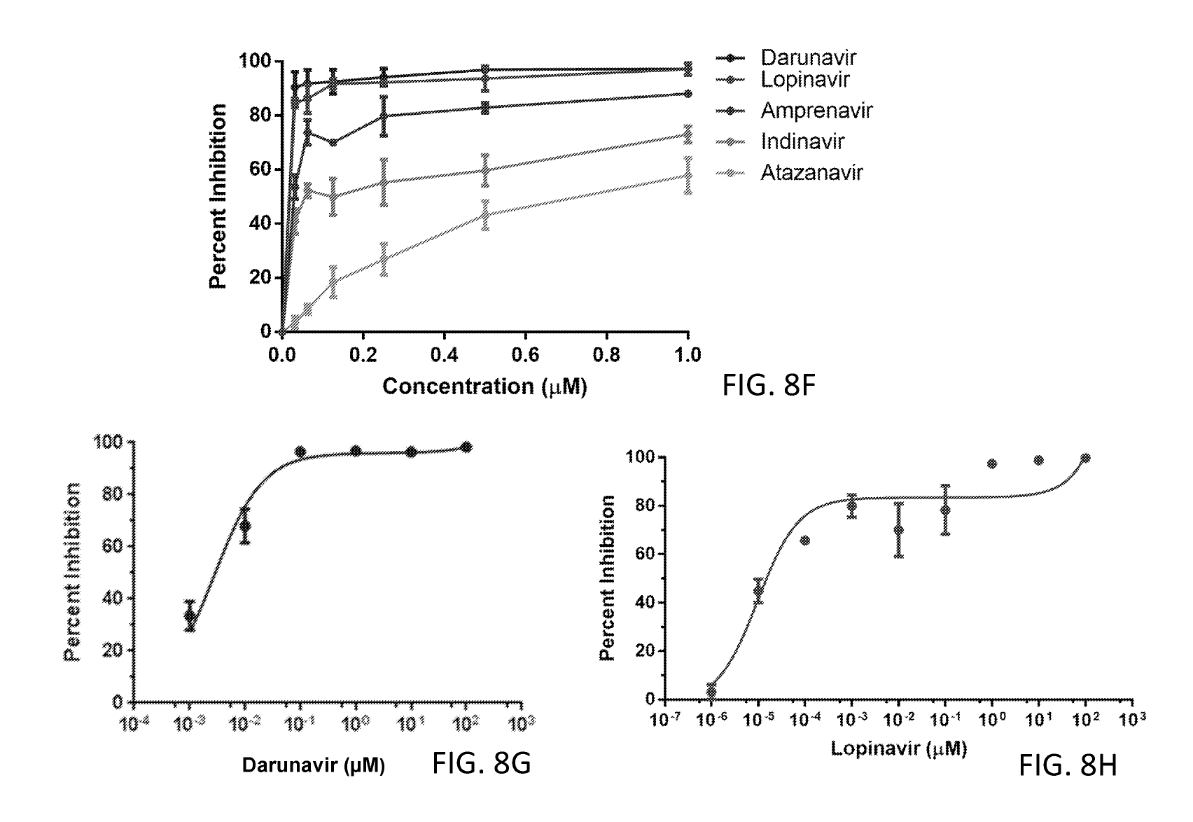

VSV-G-pseudotyped HERV-K viral particles allowed efficient infection of most cell types, while replication of HERV-K inside the cells is not altered by VSV-G protein. To normalize the amount of viral particles used for infection, recombinant HIV-1 RT was used as equivalent of HERV-K RT to generate a standard curve for the PERT assay. HERV-K viral particles were then expressed as the amount of equivalent RT. Replication of HERV-K was determined by quantitative polymerase chain reaction (qPCR) for the gag gene. As shown in FIG. 8A, left, without pseudotyping with VSV-G, there was only a 2-fold increase in gag expression after 6-days of infection with 80 μg HERV-K in HeLa cells, while VSV-G pseudotyped HERV-K had a 6-fold increase. Similar results were obtained with infection of 293T cells (FIG. 8A, right). RT inhibitors were added immediately after the inoculation of virus. The concentration of inhibitors was chosen such that they did not cause toxicity to HeLa cells as determined by a cell viability assay. After 6 days of infection, HERV-K gag RNA was determined by qPCR as a measurement of HERV-K replication. Abacavir (FIG. 8B) and Zidovudine (FIG. 8C) both inhibited HERV-K replication in a dose-dependent manner, with IC90 of 0.175 μM and 0.070 μM respectively.

HERV-K RT displays 21.5% sequence identity with HIV-1 RT. Comparative modeling of HERV-K RT and its complexes with NNRTIs was analyzed using HIV-1 RT complex with Efirvarenz and Nevirapine as templates. The complex with etravirine was modeled ab-initio using the others as guides. These drugs bind to an allosteric site in a hydrophobic cavity (NNRTI binding pocket) nearby the RT motif YIDD that interacts with the DNA. The modeling showed that most of the residues that line the cavity are not conserved. However, the small inhibitors efirvarenz and nevirapine still can be docked snuggly inside the cavity. The large etravirine showed steric clashes of its benzonitrile side chain and central ring amine substituent with the RT.

Although HERV-K protease has only 20% amino acid homology with HIV-1 protease, their core functional domains share similar structures. Comparative modeling of this core showed that the residues centered at the active site cavity and participating in the dimer interface are fully conserved between these proteases. The residues participating in the dimer contacts at the tip of the hairpin that forms the protease flap domain show high conservation. But HERV-K has an insertion in the N-terminal region of the flap that may impact its flexibility. This flexibility has been long recognized to play a role in inhibitor/substrate binding. At S1 (amino acids 87 to 91) and S2 (amino acids 30, 31, 52, 91) and their symmetry related S1′ and S2′ pockets, residue changes for larger residues reduce the size of the catalytic site. Most of these changes conserve hydrophobic characteristics at the site, only residue 31 changes from Asp to Val. At the S1 pocket, Leu89 and 91 replace HIV-1 smaller Val and Ile, respectively, reducing the size of this hydrophobic pocket. Val31, Leu53, Val53, and Leu91 replace Asp30, Ile47, Gly48, and Ile89 respectively reducing also the size of S2. However, the protease inhibitors Darunavir (FIGS. 8E and 8G) and Lopinavir (FIGS. 8F and 8H) readily dock to the protease catalytic site. This modeling suggests that HIV-1 proteases inhibitors should have also an inhibitory effect on HERV-K protease, although tweaking the size of the groups at these pockets could positively impact drug inhibition. The protease cleaves Gag-Pol precursor protein to form mature viral proteins for viral particle packaging. Hence, inhibition of protease would prevent viral particles from being formed. To determine the effect of protease inhibitors on HERV-K production, HeLa cells were transfected with HERV-K plasmid. HIV-1 protease inhibitors were added to the culture medium 6 hr. after the transfection. PERT assay was performed 48 hrs after transfection. As shown in FIG. 8F, all protease inhibitors significantly inhibited HERV-K viral production in a dose-dependent manner. Lopinavir and Darunavir showed the highest efficacy, with IC90 in the 0.1 μM range. To further determine the efficacy of Lopinavir and Darunavir at lower dosages, more extensive dose-response curves were conducted. Darunavir (FIG. 8G) and Lopinavir (FIG. 8H) both inhibited HERV-K protease in a dose-dependent manner, with IC90 of 0.071 μM and 0.651 μM, respectively (FIGS. 8G and 8H).

Currently there are three FDA-approved integrase inhibitors: Dolutegravir, Elvitegravir, and Raltegravir. The effect of Raltegravir was tested on HERV-K replication. VSV-G pseudotyped HERV-K was used to infect HeLa cells. Raltegravir was added immediately after viral inoculation. After 6 days of infection, HERV-K gag gene was determined by qPCR. As shown in FIG. 8D, Raltegravir inhibited the replication of HERV-K in a dose-dependent manner, with an 1090 of 0.075 μM

Comparative modeling of the HERV-K integrase (IN) active site using simian prototype foamy virus (PFV) integrase core domain as template (18% identity) showed the conservation of the three carboxylated coordination of a pair of divalent metal cations (Mg2+ or Mn2+) that assist the nucleophilic substitution of the viral DNA. Elvitegravir and raltegravir inhibit PFV INT. The only active site difference between these enzymes, a proline to serine (Ser729) residue, participates in these drugs recognition; however, the small change produced by the substitution could have little effect on the inhibitor recognition.

The foregoing examples have been presented for purposes of illustration and description. Furthermore, the description is not intended to limit this disclosure to the form disclosed herein. Consequently, variations and modifications commensurate with the above teachings, and the skill or knowledge of the relevant art, are within the scope of the present invention. The embodiments described hereinabove are further intended to explain the best mode known for practicing this disclosure and to enable others skilled in the art to utilize this disclosure in such, or other, embodiments and with various modifications required by the particular applications or uses of the present invention. It is intended that the appended claims be construed to include alternative embodiments to the extent permitted by the prior art.

What is claimed is:

1-16. (canceled)

17. A method of treating or preventing amyotrophic lateral sclerosis (ALS) in a subject, comprising administering to a subject diagnosed with ALS, or a subject at risk for developing ALS, one or more compounds that reduce the viral load of Human Endogenous Retrovirus Type K (HERV-K) in the subject.

18. The method of claim 17, wherein the one or more compounds comprise an antiretroviral drug.

19. The method of claim 17, wherein the one or more compounds comprise at least one compound selected from the group consisting of a reverse transcriptase inhibitor, a protease inhibitor, and an integrase inhibitor.

20. The method of claim 17, wherein at least one of the one or more compounds binds to the HERV-K genome, or to a HERV-K mRNA molecule.

21. The method of claim 20, wherein binding of the one or more compounds to the HERV-K genome inhibits transcription of one or more HERV-K nucleic acid sequences.

22. The method of claim 20, wherein binding of the one or more compounds to the HERV-K genome inhibits binding of a protein selected from the group consisting of an RNA polymerase, a transcription factor, and a repressor protein, to the proteins' binding site in the HERV-K genome.

23. The method of claim 17, wherein the one or more compounds comprise a therapeutic oligonucleotide (tON).

24. The method of claim 23, wherein the tON comprises a sequence selected from the group consisting of SEQ ID NO:6, SEQ ID NO:7, SEQ ID NO:8, SEQ ID NO:9, and SEQ ID NO:10.

25. A method of treating or preventing amyotrophic lateral sclerosis (ALS) in a subject, comprising administering to a subject diagnosed with ALS, or a subject at risk for developing ALS, one or more compounds that reduce the level of at least one HERV-K protein in the subject.

26. The method of claim 25, wherein the one or more compounds comprise an antiretroviral drug.

27. The method of claim 25, wherein the one or more compounds comprise at least one compound selected from the group consisting of a reverse transcriptase inhibitor, a protease inhibitor, and an integrase inhibitor.

28. The method of claim 25, wherein at least one of the one or more compounds binds to the HERV-K genome, or to a HERV-K mRNA molecule.

29. The method of claim 28, wherein binding of the one or more compounds to the HERV-K genome inhibits transcription of one or more HERV-K nucleic acid sequences.

30. The method of claim 28, wherein binding of the one or more compounds to the HERV-K genome inhibits binding of a protein selected from the group consisting of an RNA polymerase, a transcription factor, and a repressor protein, to the proteins' binding site in the HERV-K genome.

31. The method of claim 25, wherein the one or more compounds comprise a therapeutic oligonucleotide (tON).

32. The method of claim 31, wherein the tON comprises a sequence selected from the group consisting of SEQ ID NO:6, SEQ ID NO:7, SEQ ID NO:8, SEQ ID NO:9, and SEQ ID NO:10.

33. An isolated therapeutic oligonucleotide (tON) that binds to a HERV-K nucleic acid molecule selected from the group consisting of a HERV-K genome, and a HERV-K mRNA molecule, wherein binding of the tON to the HERV-K nucleic acid molecule reduces the viral load in a subject, and/or reduces the level of one or more HERV-K proteins.

34. The isolated tON of claim 33, wherein binding of the tON to the HERV-K genome inhibits binding of a protein selected from the group consisting of an RNA polymerase, a transcription factor, and a repressor protein, to the proteins' binding site in the HERV-K genome.

35. The method of claim 33, wherein the one or more HERV-K envelope protein comprises a HERV-K envelope protein.

36. The method of claim 33, wherein the tON comprises a sequence selected from the group consisting of SEQ ID NO:6, SEQ ID NO:7, SEQ ID NO:8, SEQ ID NO:9, and SEQ ID NO:10.
